SEQUENCE LISTING (56 sequences)

SEQ ID NO: 1 - DNA, 3506 bp, Homo sapiens
  CDS (409)..(2139)
acttcctgccgggctgcgggcgcctgagcgctcttcagcgtttgcgcggcggctgcgcgtctctctcggctcccgcttcctttgaccgcctcccccccccggcccggcggcgcccgcctcctccacggccactccgcctcttccctcccttcgtcccttcttcctctcccttttttccttcttccttcccctcctcgccgccaccgcccaggaccgccggccgggggacgagtccggagcagcagccagagtttattaaccacttaacctctcagaactgaacaaagacaacattgttcctggaacgccctctttttaaaaaaggtagaactttagacttcatagcactgaattaacctgcactgaaagctgtttacctgcatttgttcacttttgttgaaagtgaccatgtctcaagttcaagtgcaagttcagaacccatctgctgctctctcagggagccaaatactgaacaagaaccagtctcttctctcacagcctttgatgagtattccttctactactagctctctgccctctgaaaatgcaggtagacccattcaaaactctgctttaccctctgcatctattacatccaccagtgcagctgcagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcagcggcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctgaaccttttctggccatgaaccattataaaatcccaacatatatactgaaaatactgaaactgctttgaaaatttggaatttctgatacctccagtgggccgagagacacggtgggtaaaggatgtgggcagcagcagggaagacaacagaaacacaaggaggcggctgtggccggctggactgtgctggggtttgttgtgatggccactcggtgacctggcggtccctacgcaatagcagctgcctgtggggaagaagggctgcccagccagctggttctcccgggacaccagcagatccacaccctgggcacctccgtgtttggtcttttttttcccctgtgtgaaagaagaaacggcacgaccccttctcaagctggctcactcagacacattgggacaaaccctggacagccatgccagagagaggcctttgaccggccccagagctaaaagcaccagagaaaatcaaatgcttcctactcagcgtgacccaacttttctagtgtgccacggccccaccacctcctgcagtacccacaccatcaccactgctttctcttccaacagtgatctgtattcttagtttcattattttcttttgattgatatgacactatataaaattttcatttgagaatttctcaattgtatctagttaaatagcacagtttggaaacttgtctgagactgactttatcaataatctaaccgacaaagatcatatccatgtgtatgtggttagacatttttatttcattgactaacccaggacagtttcagtgatgcaaattgtgtgccctctggttcagctgaaacagtcctggactttcaaaaaccttgaataagtctcccacagttgtataaattggacaatttaggaattttaaactttagatgatcatttggttccatttttatttcatttttatttttgttaatgcaaacaggacttaaatgaactttgatctctgttttaaagattattaaaaaacattgtgtatctatacatatggctcttgaggacttagctttcactacactacaggatatgatctccatgtagtccatataaacctgcagagtgattttccagagtgctcgatactgttaattacatctccattagggctgaaaagaatgacctacgtttctgtatacagctgtgttgcttttgatgttgtgttactgtacacagaagtgtgtgcactgaggctctgcgtgtggtccgtatggaaaacctggtagccctgcgagttaagtactgcttccattcattgtttacgctggaatttttctccccatggaatgtaagtaaaacttaagtgtttgtcatcaataaatggtaatactaaaaaaaaaaaaa

SEQ ID NO: 2 - protein, 577 aa, Homo sapiens
MSQVQVQVQNPSAALSGSQILNKNQSLLSQPLMSIPSTTSSLPSENAGRPIQNSALPSASITSTSAAAESITPTVELNALCMKLGKKPMYKPVDPYSRMQSTYNYNMRGGAYPPRYFYPFPVPPLLYQVELSVGGQQFNGKGKTRQAAKHDAAAKALRILQNEPLPERLEVNGRESEEENLNKSEISQVFEIALKRNLPVNFEVARESGPPHMKNFVTKVSVGEFVGEGEGKSKKISKKNAAIAVLEELKKLPPLPAVERVKPRIKKKTKPIVKPQTSPEYGQGINPISRLAQIQQAKKEKEPEYTLLTERGLPRRREFVMQVKVGNHTAEGTGTNKKVAKRNAAENMLEILGFKVPQRQPTKPALKSEEKTPIKKPGDGRKVTFFEPGSGDENGTSNKEDEFRMPYLSHQQLPAGILPMVPEVAQAVGVSQGHHTKDFTRAAPNPAKATVTAMIARELLYGGTSPTAETILKNNISSGHVPHGPLTRPSEQLDYLSRVQGFQVEYKDFPKNNKNEFVSLINCSSQPPLISHGIGKDVESCHDMAALNILKLLSELDQQSTEMPRTGNGPMSVCGRC

SEQ ID NO: 3 - DNA, 3217 bp, Homo sapiens
  CDS (363)..(1850)
acttcctgccgggctgcgggcgcctgagcgctcttcagcgtttgcgcggcggctgcgcgtctctctcggctcccgcttcctttgaccgcctcccccccccggcccggcggcgcccgcctcctccacggccactccgcctcttccctcccttcgtcccttcttcctctcccttttttccttcttccttcccctcctcgccgccaccgcccaggaccgccggccgggggacgagtccggagcagcagccagagtttattaaccacttaacctctcagaactgaacaaagacaacattgttcctggaacgccctctttttaaaaaagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcagcggcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctgaaccttttctggccatgaaccattataaaatcccaacatatatactgaaaatactgaaactgctttgaaaatttggaatttctgatacctccagtgggccgagagacacggtgggtaaaggatgtgggcagcagcagggaagacaacagaaacacaaggaggcggctgtggccggctggactgtgctggggtttgttgtgatggccactcggtgacctggcggtccctacgcaatagcagctgcctgtggggaagaagggctgcccagccagctggttctcccgggacaccagcagatccacaccctgggcacctccgtgtttggtcttttttttcccctgtgtgaaagaagaaacggcacgaccccttctcaagctggctcactcagacacattgggacaaaccctggacagccatgccagagagaggcctttgaccggccccagagctaaaagcaccagagaaaatcaaatgcttcctactcagcgtgacccaacttttctagtgtgccacggccccaccacctcctgcagtacccacaccatcaccactgctttctcttccaacagtgatctgtattcttagtttcattattttcttttgattgatatgacactatataaaattttcatttgagaatttctcaattgtatctagttaaatagcacagtttggaaacttgtctgagactgactttatcaataatctaaccgacaaagatcatatccatgtgtatgtggttagacatttttatttcattgactaacccaggacagtttcagtgatgcaaattgtgtgccctctggttcagctgaaacagtcctggactttcaaaaaccttgaataagtctcccacagttgtataaattggacaatttaggaattttaaactttagatgatcatttggttccatttttatttcatttttatttttgttaatgcaaacaggacttaaatgaactttgatctctgttttaaagattattaaaaaacattgtgtatctatacatatggctcttgaggacttagctttcactacactacaggatatgatctccatgtagtccatataaacctgcagagtgattttccagagtgctcgatactgttaattacatctccattagggctgaaaagaatgacctacgtttctgtatacagctgtgttgcttttgatgttgtgttactgtacacagaagtgtgtgcactgaggctctgcgtgtggtccgtatggaaaacctggtagccctgcgagttaagtactgcttccattcattgtttacgctggaatttttctccccatggaatgtaagtaaaacttaagtgtttgtcatcaataaatggtaatactaaaaaaaaaaaaa

SEQ ID NO: 4 - protein, 496 aa, Homo sapiens
MKLGKKPMYKPVDPYSRMQSTYNYNMRGGAYPPRYFYPFPVPPLLYQVELSVGGQQFNGKGKTRQAAKHDAAAKALRILQNEPLPERLEVNGRESEEENLNKSEISQVFEIALKRNLPVNFEVARESGPPHMKNFVTKVSVGEFVGEGEGKSKKISKKNAAIAVLEELKKLPPLPAVERVKPRIKKKTKPIVKPQTSPEYGQGINPISRLAQIQQAKKEKEPEYTLLTERGLPRRREFVMQVKVGNHTAEGTGTNKKVAKRNAAENMLEILGFKVPQRQPTKPALKSEEKTPIKKPGDGRKVTFFEPGSGDENGTSNKEDEFRMPYLSHQQLPAGILPMVPEVAQAVGVSQGHHTKDFTRAAPNPAKATVTAMIARELLYGGTSPTAETILKNNISSGHVPHGPLTRPSEQLDYLSRVQGFQVEYKDFPKNNKNEFVSLINCSSQPPLISHGIGKDVESCHDMAALNILKLLSELDQQSTEMPRTGNGPMSVCGRC

SEQ ID NO: 5 - DNA, 3142 bp, Homo sapiens
  CDS (288)..(1775)
acttcctgccgggctgcgggcgcctgagcgctcttcagcgtttgcgcggcggctgcgcgtctctctcggctcccgcttcctttgaccgcctcccccccccggcccggcggcgcccgcctcctccacggccactccgcctcttccctcccttcgtcccttcttcctctcccttttttccttcttccttcccctcctcgccgccaccgcccaggaccgccggccgggggacgagtccggagcagcagccagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcagcggcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctgaaccttttctggccatgaaccattataaaatcccaacatatatactgaaaatactgaaactgctttgaaaatttggaatttctgatacctccagtgggccgagagacacggtgggtaaaggatgtgggcagcagcagggaagacaacagaaacacaaggaggcggctgtggccggctggactgtgctggggtttgttgtgatggccactcggtgacctggcggtccctacgcaatagcagctgcctgtggggaagaagggctgcccagccagctggttctcccgggacaccagcagatccacaccctgggcacctccgtgtttggtcttttttttcccctgtgtgaaagaagaaacggcacgaccccttctcaagctggctcactcagacacattgggacaaaccctggacagccatgccagagagaggcctttgaccggccccagagctaaaagcaccagagaaaatcaaatgcttcctactcagcgtgacccaacttttctagtgtgccacggccccaccacctcctgcagtacccacaccatcaccactgctttctcttccaacagtgatctgtattcttagtttcattattttcttttgattgatatgacactatataaaattttcatttgagaatttctcaattgtatctagttaaatagcacagtttggaaacttgtctgagactgactttatcaataatctaaccgacaaagatcatatccatgtgtatgtggttagacatttttatttcattgactaacccaggacagtttcagtgatgcaaattgtgtgccctctggttcagctgaaacagtcctggactttcaaaaaccttgaataagtctcccacagttgtataaattggacaatttaggaattttaaactttagatgatcatttggttccatttttatttcatttttatttttgttaatgcaaacaggacttaaatgaactttgatctctgttttaaagattattaaaaaacattgtgtatctatacatatggctcttgaggacttagctttcactacactacaggatatgatctccatgtagtccatataaacctgcagagtgattttccagagtgctcgatactgttaattacatctccattagggctgaaaagaatgacctacgtttctgtatacagctgtgttgcttttgatgttgtgttactgtacacagaagtgtgtgcactgaggctctgcgtgtggtccgtatggaaaacctggtagccctgcgagttaagtactgcttccattcattgtttacgctggaatttttctccccatggaatgtaagtaaaacttaagtgtttgtcatcaataaatggtaatactaaaaaaaaaaaaa

SEQ ID NO: 6 - DNA, 3348 bp, Homo sapiens
  CDS (494)..(1981)
acttcctgccgggctgcgggcgcctgagcgctcttcagcgtttgcgcggcggctgcgcgtctctctcggctcccgcttcctttgaccgcctcccccccccggcccggcggcgcccgcctcctccacggccactccgcctcttccctcccttcgtcccttcttcctctcccttttttccttcttccttcccctcctcgccgccaccgcccaggaccgccggccgggggacgagtccggagcagcagccagcagcagccaggtggagttttgctcttgtcgcccaggctggagtgcagtggcgtgatctcggctcactgcaacctccacctcccaggtcagcgattttcccacttcagcctcccgataagctgagattacagagtttattaaccacttaacctctcagaactgaacaaagacaacattgttcctggaacgccctctttttaaaaaagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcagcggcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctgaaccttttctggccatgaaccattataaaatcccaacatatatactgaaaatactgaaactgctttgaaaatttggaatttctgatacctccagtgggccgagagacacggtgggtaaaggatgtgggcagcagcagggaagacaacagaaacacaaggaggcggctgtggccggctggactgtgctggggtttgttgtgatggccactcggtgacctggcggtccctacgcaatagcagctgcctgtggggaagaagggctgcccagccagctggttctcccgggacaccagcagatccacaccctgggcacctccgtgtttggtcttttttttcccctgtgtgaaagaagaaacggcacgaccccttctcaagctggctcactcagacacattgggacaaaccctggacagccatgccagagagaggcctttgaccggccccagagctaaaagcaccagagaaaatcaaatgcttcctactcagcgtgacccaacttttctagtgtgccacggccccaccacctcctgcagtacccacaccatcaccactgctttctcttccaacagtgatctgtattcttagtttcattattttcttttgattgatatgacactatataaaattttcatttgagaatttctcaattgtatctagttaaatagcacagtttggaaacttgtctgagactgactttatcaataatctaaccgacaaagatcatatccatgtgtatgtggttagacatttttatttcattgactaacccaggacagtttcagtgatgcaaattgtgtgccctctggttcagctgaaacagtcctggactttcaaaaaccttgaataagtctcccacagttgtataaattggacaatttaggaattttaaactttagatgatcatttggttccatttttatttcatttttatttttgttaatgcaaacaggacttaaatgaactttgatctctgttttaaagattattaaaaaacattgtgtatctatacatatggctcttgaggacttagctttcactacactacaggatatgatctccatgtagtccatataaacctgcagagtgattttccagagtgctcgatactgttaattacatctccattagggctgaaaagaatgacctacgtttctgtatacagctgtgttgcttttgatgttgtgttactgtacacagaagtgtgtgcactgaggctctgcgtgtggtccgtatggaaaacctggtagccctgcgagttaagtactgcttccattcattgtttacgctggaatttttctccccatggaatgtaagtaaaacttaagtgtttgtcatcaataaatggtaatactaaaaaaaaaaaaa

SEQ ID NO: 7 - DNA, 2857 bp, Mus musculus
  CDS (324)..(1784)
cgggcgcggcccctcccccgtcacttcctgccaggctgcgggccccgagccgctcttcagcgtttgcgctggctgtcgtcgcgtctgtgtgcgctcccccttcttctgagccccggcctggcggcgcccgccttcgcctccgccactccgcctcttccctcctctggtcggcccctttttcctcgccgtcttcacttgcttcttcacctcctcgccgccgcccaagaccgccggccccgggacgagctctggggaagcagccagaaagtatagcttctaccattgagctcaatgcactgtgtgtgaaactggaaagaaaaccaatgtataagcccgtggaccctcactctcggatgcagtccacctacagctatggcatgcgtggaggtgcctatccccccagatacttttacccatttccagtcccacctttactctaccaagttgagctctccgtgggcggacagcagtttaatgggaaaggaaagatgagaccacccgtgaaacacgatgcccctgcccgtgcgctgaggactctgcagagtgaacccctgccagaaaggttggaggtaaatggaagagaagcagaggaagaaaacctcaataaatcggaaataagccaagtgtttgaaattgcgctgaagcggaatttgcctgtgaattttgaggtggcccgggagagtggcccaccacacatgaagaactttgtgaccagggtttcagttggggaatttgtaggggaaggagaagggaaaagcaagaagatctccaagaagaatgcggccagggctgttctggagcagcttaggaggctgccacccctccctgctgtggagcgagtgaagcccagaatcaagaagaaaagtcagcccacctgcaagacagccccggattatggccaagggatgaatcctattagtagacttgcacagatccagcaggcaaaaaaggagaaggagccagagtacatgctccttacagaacgaggtcttccacgtcgcagggagtttgtgatgcaggtaaaggttgggcatcacactgcagaaggagtgggtaccaataagaaggtggccaagcgtaatgctgctgagaacatgctggagatcctggggttcaaagttccccaggcgcagcctgccaagccagcactcaaatcagaagagaagactccagtaaagaaaccaggagacggaaggaaagtaacgttttttgaacctagccctggggatgaaaatggaactagtaacaaggacgaggagttcaggatgccttatcttagccatcagcagctgccagctggaattctccccatggtgccggaagttgcccaggctgtcggggttagtcaaggacaccacaccaaagatttcaccagggcagctccaaatcctgccaaggcaacggtaactgccatgatagcccgagagttgttgtacgggggcacctcgcccacagccgagaccattttaaagagtaacatctcttcaggccacgtaccccatggacctcgcactagaccctctgagcaactgtactacctttccagagcccagggattccaggttgaatacaaagattttcccaagaacaacaagaacgagtgtgtatctctcatcaactgctcctcacagccgcctctcgtcagtcatggcatcggcaaggatgtggagtcctgtcatgatatggctgcactgaacattttaaagctgctgtctgagttggaccaacagagcacagagatgccaagaacaggaaatggaccagtttcagcgtgcgggaggtgctgaaccttttctggccacaaaccattataaaacccaacatatatactgaaaatactgagaactgctttgaaaatttggaatatctgataactccagtgggccaagacatggtggataaaaatgtggcaaagacgacaagaaacttcaggtggtagccctggttgtgctggcggctagtgatgatgctgtgctctgccatccatccagacagaaaccagccccaacgcctccagttctgtttttgcatcgtgacaaagagagcacagccaattctcatgctggcttcttcagatactttgaaaaacccggacagccacaccagagaggccttatagcggccccggagctaaacggaccagagaaaaggccagtgcttcctactgcacatgactgactcagctccgccacacgtagcaccactgtaaccactgctttctcttcagtttcatttttttcctttgattgatacaacactataattttcatttcagttccttagtcgtgtctacttacctagcagtttagaaactgtcagtcatgtaactggcaaggatcacagcccggttgggtggcattctgtgcctctggcttggctgaacagttctggaattaccaccagaatccttgactccctgccccttgtataaattggacagcttaggacttttaaactttagatcaaaagatatggtcctttttaactttatttttaaggagcagactttaaaatgagccctgacctttacccattataacagaatttgtcaaaaggagtgtttcttgaggaggtagcttttttttaccacactacaggacattacctgtaggcccagaagactacaggctggtgtccctagagggcccaatacagtcaattccaacctctaagtcggggaaaggtgacaggtttcctggtgctggtgtgcacaggggcaggcaggtcagctggcctggggaagagcattgtggctcctagtgcagccctgcttccactcttggtttagctggaaccttcccactcatggaatataagtaaactcactttctttgtcaccaataaatggtaatactaa

SEQ ID NO: 8 - protein, 487 aa, Mus musculus
MYKPVDPHSRMQSTYSYGMRGGAYPPRYFYPFPVPPLLYQVELSVGGQQFNGKGKMRPPVKHDAPARALRTLQSEPLPERLEVNGREAEEENLNKSEISQVFEIALKRNLPVNFEVARESGPPHMKNFVTRVSVGEFVGEGEGKSKKISKKNAARAVLEQLRRLPPLPAVERVKPRIKKKSQPTCKTAPDYGQGMNPISRLAQIQQAKKEKEPEYMLLTERGLPRRREFVMQVKVGHHTAEGVGTNKKVAKRNAAENMLEILGFKVPQAQPAKPALKSEEKTPVKKPGDGRKVTFFEPSPGDENGTSNKDEEFRMPYLSHQQLPAGILPMVPEVAQAVGVSQGHHTKDFTRAAPNPAKATVTAMIARELLYGGTSPTAETILKSNISSGHVPHGPRTRPSEQLYYLSRAQGFQVEYKDFPKNNKNECVSLINCSSQPPLVSHGIGKDVESCHDMAALNILKLLSELDQQSTEMPRTGNGPVSACGRC

SEQ ID NO: 9 - protein, 1026 aa, Drosophila melanogaster [sequence omitted]

SEQ ID NO: 10 - protein, 705 aa, Caenorhabditis elegans
MQAVFETTLTQKMDGVMIVQETTTDLADTLENASISAEKSEQKPERLHPQHWCGQHKFEADSPTNFYDYTNAKEKEKSAMCRVAEIARFNKLRHVYNLQDESGPAHKKLFTVKLVLTEAETFEGSGTSIKRAQQASAEAALKGTKLPLPTEKPTKKRINDTTKPHRVLQNVCRTLQYQMPNYISCNPPVYPDPGCPLPEHILLPLESMALYAPPFPTLPIDPARPQGPKLQAVIVNINGKSIATGIGETYPLAKQDAAAKALAVLSPLLREHQNGSDNGFGKENIPVHKQKSVISDIHEKAYQLKVNVVFEVLKEEGPPHDRQYVVRCAFVTSGNVVKAEAVGKGKKKKSAQQEACTQLLATVEHLTPENNPVALATNVCKTQKKLAAMNREPKRKTIVKDKKMDPLYGHQINPVSRLIQVTQAKSKEHPTFELVAEHGVSKYKEFIIQVKYGDDVQEGKGPNKRLAKRAAAEAMLESIGFVKPLPPPGKSLLKKMIDCDPSLPEISHWTGPPPTAVSVSTSEPDTSEAAQLSPEQTDISEKRELSPDTEKRRVTFNSQVHACPPPGDQDYPNSIVQSLKKDAIVEGKIRRLKRSKENRRALTAEQIVELSERAQSYLQTKNTTIQSSQSSSAHHHLEQLSDFFKFSLQYTSFPQVGIDQHFTIVSIGLEAPLVGHGTGCSTTEADENAALDAIAKLKELSASKT

SEQ ID NO: 11 - protein, 101 aa, Homo sapiens
GFKVPQRQPTKPALKSEEKTPIKKPGDGRKVTFFDPGSGDENGTSNKEDEFRLPYLSHQQLPAGILPMVPEVAQAVGVSQGHHTKDFTRAAPNPAKATVTA

SEQ ID NO: 12 - protein, 98 aa, Homo sapiens
KEKTKTKKPGTKTKSSSPVKKSDGKSKPLAASPKPAGLKESSDKVSRVASPKKKESVEKAAKPTTTPEVKAARGEEKDKETKNAANASASKSAKTATA

SEQ ID NO: 13 - DNA, 16 bp, Artificial
  oligonucleotide
agcttaattagctgac

SEQ ID NO: 14 - DNA, 16 bp, Artificial
  oligonucleotide
agctgtcagctaatta

SEQ ID NO: 15 - DNA, 31 bp, Artificial
  oligonucleotide
cctggatccgaaagtatagcttctaccattg

SEQ ID NO: 16 - DNA, 36 bp, Artificial
  oligonucleotide
tacataagcttctagatggccagaaaaggttcagca

SEQ ID NO: 17 - DNA, 27 bp, Artificial
  oligonucleotide
ggatgaatcctattagtagacttgcac

SEQ ID NO: 18 - DNA, 29 bp, Artificial
  oligonucleotide
gctctagattcaaagttccccaggcgcag

SEQ ID NO: 19 - DNA, 29 bp, Artificial
  oligonucleotide
tttaagcttctcagagggtctagtgcgag

SEQ ID NO: 20 - DNA, 22 bp, Artificial
  oligonucleotide
caatgtataagcccgtggaccc

SEQ ID NO: 21 - DNA, 36 bp, Artificial
  oligonucleotide
aaaaagcttgtgcaagtctactaataggattcatcc

SEQ ID NO: 22 - DNA, 18 bp, Artificial
  oligonucleotide
atagcccgagagttgttg

SEQ ID NO: 23 - DNA, 36 bp, Artificial; identical to SEQ ID NO: 16
  oligonucleotide

SEQ ID NO: 24 - DNA, 29 bp, Artificial
  oligonucleotide
tacatgtcgacttcctgccrggctgcggg

SEQ ID NO: 25 - DNA, 44 bp, Artificial
  oligonucleotide
tacaatctagattatcagcggccgcacctcccacacacagacat

SEQ ID NO: 26 - DNA, 41 bp, Artificial
  oligonucleotide
tacataagcttaagccaccatggtcaaagttccccaggcgc

SEQ ID NO: 27 - DNA, 40 bp, Artificial
  oligonucleotide
tacaatctagagcggccgcgctcagagggtctagtgcgag

SEQ ID NO: 28 - DNA, 28 bp, Artificial
  oligonucleotide
acaggatcccttgtgacgaggggtcgtt

SEQ ID NO: 29 - DNA, 28 bp, Artificial
  oligonucleotide
acaggatccccaaaactcttgccttatg

SEQ ID NO: 30 - DNA, 28 bp, Artificial
  oligonucleotide
acaggatccccaaaactcttgccttatg

SEQ ID NO: 31 - DNA, 28 bp, Artificial
  oligonucleotide
acaggatcccaaaattccctggccttcc

SEQ ID NO: 32 - DNA, 28 bp, Artificial
  oligonucleotide
acaggatcccttgtgacgaggggtcgtt

SEQ ID NO: 33 - DNA, 28 bp, Artificial
  oligonucleotide
acaggatcccgtaattttggctgacctg

SEQ ID NO: 34 - DNA, 28 bp, Artificial
  oligonucleotide
acaggatcccaaaattccctggccttcc

SEQ ID NO: 35 - DNA, 28 bp, Artificial
  oligonucleotide
acaggatcccattagcctgtctctcagt

SEQ ID NO: 36 - DNA, 28 bp, Artificial
  oligonucleotide
acaggatcccttgtgacgaggggtcgtt

SEQ ID NO: 37 - DNA, 28 bp, Artificial
  oligonucleotide
acaggtaccatgggtgcgagagcgtcag

SEQ ID NO: 38 - DNA, 28 bp, Artificial
  oligonucleotide
acaggtaccatgggtgcgagagcgtcag

SEQ ID NO: 39 - DNA, 29 bp, Artificial
  oligonucleotide
acaggtaccatgcctatagtgcagaacat

SEQ ID NO: 40 - DNA, 29 bp, Artificial
  oligonucleotide
acaggtaccatgcctatagtgcagaacat

SEQ ID NO: 41 - DNA, 29 bp, Artificial
  oligonucleotide
acaggtaccatgcctatagtgcagaacat

SEQ ID NO: 42 - DNA, 28 bp, Artificial
  oligonucleotide
acaggtaccatgggtgcgagagcgtcag

SEQ ID NO: 43 - DNA, 28 bp, Artificial
  oligonucleotide
acaggtaccatggctgaagcaatgagcc

SEQ ID NO: 44 - DNA, 28 bp, Artificial
  oligonucleotide
acaggtaccatgcagagaggcaatttta

SEQ ID NO: 45 - DNA, 28 bp, Artificial
  oligonucleotide
acaggtaccatgcttcagagcagaccag

SEQ ID NO: 46 - DNA, 30 bp, Artificial
  oligonucleotide
gatgctcgagatgaaacttggaaaaaaacc

SEQ ID NO: 47 - DNA, 41 bp, Artificial
  oligonucleotide
cacatctagatcatttattcagcggccgcactgagcagcgt

SEQ ID NO: 48 - DNA, 22 bp, Artificial
  oligonucleotide
ctgagcctgggagctctctggc

SEQ ID NO: 49 - DNA, 42 bp, Artificial
  oligonucleotide
tccagtgatttttttctccatgcttgcccatactatatgttt

SEQ ID NO: 50 - DNA, 23 bp, Artificial
  oligonucleotide
tcattgccactgtcttctgctct

SEQ ID NO: 51 - DNA, 21 bp, Artificial
  oligonucleotide
ccgcagatcgtcccagataag

SEQ ID NO: 52 - DNA, 21 bp, Artificial
  oligonucleotide
aaatagcacagtttgcaaact

SEQ ID NO: 53 - DNA, 21 bp, Artificial
  oligonucleotide
aattctccgaacgtgtcacgt

SEQ ID NO: 54 - DNA, 20 bp, Artificial
  oligonucleotide
tttaccagggcagctccgaa

SEQ ID NO: 55 - DNA, 26 bp, Artificial
  oligonucleotide
caactcagacagcaactttaagatgt

SEQ ID NO: 56 - DNA, 575 bp, Artificial
  unknown
aagcttggctgcaggtcgacggatccccccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgnacccgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcmsycttaaaattatgttttaaaatggactatcatatgcttaccgtaacttgaaagtatttcgatttcttggctttatatatcttgtggaaaggacgaaacaccgtaagggtcaacaggcttatacattggttgaagcttgagccgatgtataagcctgttgatccttattttttt

(19) United States
(12) Patent Application Publication
(10) Pub. No.: US 2004/0241845 A1
(43) Pub. Date: Dec. 2, 2004

(54) Mammalian staufen and use thereof

(76) Inventors: Luc Desgroseillers, St-Bruno (CA); Andrew Mouland, Montreal (CA); Laurent Chatel-Chaix, Montreal (CA); Jean-Francois Clement, Montreal (CA)

Correspondence Address: GOUDREAU GAGE DUBUC, 800 PLACE VICTORIA, SUITE 3400, MONTREAL, QUEBEC, H4Z 1E9, CA

(21) Appl. No.: 10/820,060

(22) Filed: Apr. 8, 2004

Related U.S. Application Data
(63) Continuation-in-part of application No. 09/316,048, filed on May 21, 1999.

(30) Foreign Application Priority Data
Aug. 22, 1998 (CA) 2,238,656

(51) Int. Cl. A61K 48/00; C07H 21/02

(52) U.S. Cl. 435/375; 514/44; 536/23.1

(57) ABSTRACT

The present invention relates to mammalian Staufen, a double-stranded RNA-binding protein involved in mRNA transport and localization. The invention further relates to the demonstration of the association of a RNA-binding protein with the rough endoplasmic reticulum (RER), implicating Staufen and related proteins in the transport of RNA to its site of translation. Broadly, the invention therefore relates to transport and translation of RNA. More specifically, the present invention relates to human and mouse Staufen proteins and to the modulation of transport of RNA to the RER by these proteins. The present invention also relates to isolated nucleic acid molecules encoding mammalian Staufen, as well as vectors and host cells harboring same. In addition, the present invention relates to screening assays for identifying modulators of Staufen activity and to the identification of mutants thereof which abrogate their interaction with RER. Furthermore, the present invention relates to the use of the double-stranded RNA binding activity of Staufen as a means to target proteins into virions. The invention in addition relates to the incorporation of Staufen into RNA viruses and the use of overexpression or decrease expression of Staufen by RNA interference to significantly decrease the infectivity thereof. More particularly, the present invention relates to a novel and broad class of molecules which can be used as carriers to target molecules into virions of RNA viruses and to decrease infectivity of a wide variety of RNA viruses including retroviruses.

### CROSS REFERENCES TO RELATED APPLICATIONS

The present application is a continuation-in-part of U.S. application Ser. No. 09/316,048.

### FIELD OF THE INVENTION

The present invention relates to mammalian Staufen, a double-stranded RNA-binding protein involved in mRNA transport and localization. The invention further relates to the demonstration of the association of a RNA-binding protein with the rough endoplasmic reticulum (RER), implicating Staufen and related proteins in the transport of RNA to its site of translation. Broadly, the invention therefore relates to transport and translation of RNA. More specifically, the present invention relates to human and mouse Staufen proteins and to the modulation of transport of RNA to the RER by these proteins. The present invention also relates to isolated nucleic acid molecules encoding mammalian Staufen, as well as vectors and host cells harboring same. In addition, the present invention relates to screening assays for identifying modulators of Staufen activity and to the identification of mutants thereof which abrogate their interaction with RER. Furthermore, the present invention relates to the use of the double-stranded RNA binding activity of Staufen as a means to target proteins into virions. The invention in addition relates to the incorporation of Staufen into RNA viruses, to its interaction with pr55^{GAG} and to the modulation of Staufen expression as a way of significantly decreasing the infectivity thereof. More particularly, the present invention relates to a novel and broad class of molecules which can be used as carriers to target molecules in to virions of RNA viruses and to decrease infectivity of a wide variety of RNA viruses including retroviruses.

### BACKGROUND OF THE INVENTION

It is now believed that the cytoskeleton is widely used to transport mRNAs between their transcription and processing sites in the nucleus and their translation and degradation sites in the cytoplasm (Pachter, J. S. (1992), Gene Exp. 2, 1-18; Bassell et al. (1997), Curr. Opin. Cell Biol. 9, 109-115; Nakielny et al. (1997), Ann. Rev. Neurosci. 20, 269-301). One consequence of the interaction between mRNAs and the cytoskeleton is to promote differential localization and/or transport of mRNAs in subcellular compartments. Indeed, examples of mRNA targeting were observed in both germinal and somatic cells throughout the animal kingdom (Wilhelm et al. (1993), J. Cell Biol. 123, 269-274; St Johnston, D. (1995), Cell 81, 161-170; Steward, O. (1997), Neuron 18, 9-12). The universal use of this mechanism is also apparent when we consider the nature of the proteins which are coded by the transported mRNAs; asymmetric localization involving mRNAs coding for cytosolic, secreted, membrane-associated or cytoskeletal proteins have all been reported. Localization of mRNAs in the cytoplasm is now considered an essential step in the regulation of gene expression and an efficient way to unevenly distribute proteins in polarized cells. In general, it is believed that mRNA localization is used to determine and/or regulate local sites of translation (Rings et al. (1994), Eur. J. Cell Biol. 63, 161-171; St Johnston, 1995, supra; Steward, 1997, supra). Indeed, ribosomes and many translational cofactors were found in association with the cytoskeletal elements, preventing both mRNAs and translation factors from being diluted by the cellular fluid (Pachter, 1992, supra). Transport and local translation of specific mRNAs has been shown to play an important role in processes such as learning and memory (Martin et al. (1997), Cell 91, 927-938), synaptic transmission (Crino et al. (1996), Neuron 17, 1173-1187; Kang et al. (1996), Science 273, 1402-1406; Gazzaley et al. (1997), J. Neurosci. 17, 2006-2017; Steward, 1997, supra; Tongiorgi et al. (1997), J. Neurosci. 17, 9492-9505), axis formation during development (reviewed in St Johnston, 1995, supra), cell motility (Kislauskis et al. (1997), J. Cell Biol. 136, 1263-1270), and asymetric cell division (Li et al. (1997), Cell 90, 437-447; Long et al. (1997), Science 177, 383-387; Takizawa et al. (1997), Nature 389,90-93; Broadus et al. (1998), Nature 391, 792-795).

The mechanisms underlying mRNA localization are not yet fully understood, mainly because of the lack of information on the principal constituents of the ribonucleoprotein complexes involved in this process. Nevertheless, it is known to involve both cis-acting signals in mRNA and trans-acting RNA-binding proteins which bind to this signal (St Johnston, 1995, supra). The signals that allow mRNAs to be recognized as a target for transport and then to be localized have been mapped within their 3′-untranslated regions (Wilhelm and Vale, 1993, supra; St Johnston, 1995, supra). In contrast, the nature of the RNA-binding proteins is still obscure. Recently, a 68 kDa protein which binds the β-actin mRNA zipcode localization domain was isolated and its transcript was cloned from chicken cDNA libraries (Ross et al. (1997), Mol. Cell. Biol. 17, 2158-2165). This protein, which binds to microfilaments, contains an. RNA-binding domain which shares strong sequence similarity with the RNP1 and RNP2 motifs. In addition, 69 kDa and 78 kDa proteins in Xenopus oocyte extracts have been shown to bind to the localization signal of Vg1 mRNA (Schwartz et al. (1992), PNAS 89, 11895-11899; Deshler et al. 1997, Science 276, 1128-1131). While the 69 kDa protein was shown to bind microtubules (Elisha et al. (1995), EMBO J. 14, 5109-5114), the 78 kDa Vera protein co-localized with a sub-domain of the smooth endoplasmic reticulum (Deshler et al., 1997, supra). However, since these proteins have not yet been characterized, their nature and function in localization remain unclear.

Genetic and molecular studies have shown that the activity of the Staufen gene product in Drosophila is necessary for the proper localization of bicoid and oskar mRNAs to the anterior and posterior cytoplasm of oocytes, respectively, and of prospero mRNA in neuroblasts (St Johnston et al. (1989), Dev. (Suppl.) 107,13-19; Ephrussi et al. (1991), Cell 66, 37-50; Kim-Ha et al. (1991), Cell 66,23-35; St Johnston et al. (1991), Cell 66, 51-63; Broadus et al., 1997, supra; Li et al., 1997, supra). Staufen is a member of the double-stranded RNA-binding protein family, and contains three copies of a domain consisting of a 65- to 68-amino acid consensus sequence which is required to bind RNAs having double-stranded secondary structures, and two copies of a short-domain, which retains the last 21 amino acids at the C-terminal end of the complete motif (St Johnston et al., 1991, supra; St Johnston et al. (1992), PNAS 89, 10979-10983). In vitro, it has been demonstrated that Staufen binds directly to bicoid and prospero mRNAs (St Johnston et al., 1992, supra; Li et al., 1997, supra). However, since Staufen seems to bind to any dsRNA in vitro, it is not clear whether or not it binds directly to these RNAs in vivo, or needs cellular co-factors which make up part of a larger ribonucleoprotein complex to localize each mRNA. Many experiments have demonstrated that the localization of oskar, prospero and bicoid mRNAs occurs through a multistep mechanism of active transport that is dependent on elements of the cytoskeleton (Erdelyi et al. (1995), Nature 377, 524-527; Pokrywka et al. (1995), Dev. Biol. 167, 363-370; St Johnston, 1995, supra; Tetzlaff et al. (1996), EMBO J. 15, 1247-1254; Broadus et al., 1997, supra).

There thus remains a need to understand the mechanisms of mRNA transport in mammals and determine the nature of both the RNAs and proteins in the RNA/protein complexes. Both Southern blot analysis of human DNA and fluorescent in situ hybridization (FISH) on human chromosomes in metaphase showed that the human gene is present as a single copy in the human genome and is localized in the middle of the long arm of chromosome 20 (DesGroseillers et al. (1996), Genomics 36, 527-529). The identification and characterization of human or another mammalian Staufen is desired as it could provide critical information in the transport, and proper localization of mRNAs in subcellular compartments.

Staufen (Stau) was originally described as a dsRNA-binding protein in Drosophila melanogaster (St Johnston et al., 1991, supra). It was further shown to specifically bind the 3′ untranslated region of the mRNA for bicoid (Ferrandon et al. (1994), Cell 79, 1221-1232), a morphogen responsible for anterior body pattern formation in the early embryo. In Drosophila, Stau's principle function is to target mRNAs for localized translation (Ferrandon et al., 1994, supra, Huang et al. (1997), J. Virol. 71, 4378): it serves to localize oskar mRNA posteriorally (Huang et al., 1997, supra) and anchors bicoidmRNA anteriorally in oocytes, and recently has been shown to localize prospero mRNA in neuroblasts (Li et al., 1997, supra; Broadus et al., 1998, supra). The human homologue (hStau) is hereinbelow further characterized and is shown to have several structural and functional domain similarities to its Drosophila counterpart (Wickham et al. (1996), Genomics 36:527).

A more thorough understanding of the structure-function relationship of mammalian Staufen is needed to better understand its function in mammalian cells. There also remains a need to better understand the dsRNA-binding activity of mammalian Staufen and to analyze the function and application thereof in cellular homoeostasy. In addition, this understanding could help characterize the important molecular determinants of Staufen from lower eukaryotes.

It would be highly desirable to be provided with means to target molecules to RNA viruses, including retroviruses, such as HIV virions. It would also be desirable to be provided with means to target molecules into such viruses and affect their structural organization and/or functional integrity and/or morphogenesis.

It would also be highly desirable to be provided with a protein, fragment or derivative thereof which permits the development of chimeric molecules that can be specifically targeted into RNA viruses in general, and more particularly retroviruses, including lantiviruses such as HIV. Such chimeric molecules could be used for the treatment of RNA virus infections, retroviral infections and lentiviral infections.

It would also be highly desirable to be provided with a therapeutic agent which permits targeting of chimeric molecules into RNA virions, as a treatment for diseases caused by such virions.

It would also be highly desirable to be provided with the identification of novel molecular determinants responsible for the incorporation of proteins into virions via their interaction with genomic RNA, for RNA genome incorporation into RNA viruses, as well as the identification of molecular determinants involved in the targeting of RNA molecules to the RER.

It would also be highly desirable to be provided with means to target RNA molecules to the RER.

It would also be very desirable to be provided with therapeutic agent molecules which interfere with the molecular determinant responsible for RNA genome incorporation into RNA virions as well as agents which interfere with the targeting of RNA molecules to the RER as such agents could have therapeutic utility for the treatment of diseases including viral diseases.

It would further be highly desirable to be provided with an assay which enables the screening and identification of molecules which modulate the interaction between the molecular determinant responsible for RNA genome incorporation into RNA virions. As well, it would be highly desirable to be provided with an assay which enables the screening and identification of molecules which modulate the targeting of RNA molecules to the RER.

It would in addition be highly desirable to be provided with a method for screening and identifying molecules which act as modulating agents of RNA genome incorporation into RNA virions and as well as a method for screening and identifying molecules which act as modulating agents for the targeting of RNA molecules to RER.

It would also be desirable to be provided with an antisense RNA or siRNA to decrease Staufen protein expression in a cell in order to modulate RNA genome incorporation into RNA virions, thereby decreasing RNA virus infectivity.

It would also be desirable to be provided with gene therapy or therapeutic vaccines to modulate RNA virus infectivity, more particularly HIV infectivity.

The present invention seeks to meet these and other needs.

The present description refers to a number of documents, the content of which is herein incorporated by reference in their entirety.

### SUMMARY OF THE INVENTION

The human homologue of the double-stranded RNA (dsRNA)-binding protein, Staufen, is shown herein to be incorporated into HIV-1 virions, and this correlates with HIV-1 genomic RNA encapsidation. It is also shown that the HIV-1 genomic RNA—but not the 1.8 or 4 kb spliced RNA species—selectively co-immunoprecipitate with Staufen. Furthermore, it is demonstrated that Staufen specifically interacts with pr55^{Gag} in a direct and RNA indenpendent manner, as shown in vitro as well as in live cells. It is further demonstrated that hStau is incorporated into clinical isolates of HIV-1, and several other retroviruses including HIV-2 and murine leukemia virus, and non-retroviral RNA viruses such as Reovirus, but is not detectable in DNA viruses.

When hStau is overexpressed, a corresponding increase of hStau in virions is observed. Strikingly, this increase in hStau incorporation into HIV-1 is accompanied by a dramatic impairment of HIV-1 infectivity. RNA interference experiments also demonstrate that the specific knockdown of Staufen gene expression results in a significant reduction in viral infectivity. This is the first demonstration of a dsRNA-binding protein within HIV-1 particles. Therefore, hStau is identified herein as an important therapeutic target in RNA virus infections such as HIV infection. These novel and unexpected finding have important implications not only in retroviral genome sorting, assembly and infectivity, but also in RNA virus therapy in general, retrovirus therapy and more particularly in HIV-1 therapy.

The present invention concerns in general mammalian Staufen and more particularly the sequence of the human and mouse Staufen proteins and nucleic acid molecules encoding same.

The present invention further relates to the demonstration that Staufen binds both dsRNA and tubulin in vitro via specific binding domains. Further, the invention relates to the localization of Staufen in the cytoplasm in association with the rough endoplasmic reticulum, implicating this protein in the targeting of RNA to its site of translation.

The present invention also relates to the demonstration that Staufen interacts with HIV pr55^{Gag} via specific domains both in vitro and in vivo.

More particularly, the present invention provides isolated polypeptides having the amino acid sequences shown in FIGS. 1A, 1B, 1C, 1D and FIG. 1′.

The present invention further relates to isolated nucleic acid molecules comprising polynucleotides which encode a Staufen polypeptide and more particularly a mammalian Staufen polypeptide. More paticularly, the present invention relates to isolated nucleic acid molecules encoding the Staufen polypeptides having the amino acid sequences shown in FIGS. 1A, 1B, 1C and 1′.

The invention in addition relates- to recombinant vectors harboring the isolated nucleic acid molecules of the present invention. More particularly, the invention relates to expression vectors which express the Staufen polypeptides of the present invention and more particularly mammalian Staufen. The present invention further relates to host cells containing such recombinant vectors or expression vectors, to methods of making such host cells, and to methods of making such vectors. Vectors and host cells expressing a mammalian Staufen sequence or fragments thereof (e.g., wild type Staufen nucleic acid sequence, Staufen siRNA, Staufen antisense) can be used in gene therapy or other methods to modulate (either increase or decrease) Staufen expression thereby decreasing virus infectivity.

The present invention further relates to the use of vaccines directed against Staufen protein as a mean of treating or preventing RNA virus infections, more particularly HIV 1 infection.

Further, the present invention provides screening assays and methods for identifying modulators of Staufen activity and especially of mammalian Staufen activity. More particularly, the present invention relates to assays and methods for screening and identifying compounds which can enhance or inhibit the RNA virion incorporation ability of Staufen and especially mammalian Staufen. In one particular embodiment of the present invention, the screening assay for identifying modulators of Staufen's incorporation ability comprises contacting cells or extracts containing Staufen and a candidate compound, assaying a cellular response or biological function of Staufen such as virion incorporation or RER targeting, for example, wherein the potential modulating compound is selected when the cellular response or Staufen's biological activity in the presence of the candidate compound is measurably different than in the absence thereof.

More particularly, the present invention further relates to methods for preventing or treating an RNA viruses infection such as a HIV infection comprising a modulation of the expression of Staufen in a cell or organism. Such methods include the use of gene therapy to overexpress a Staufen nucleic acid (or fragment thereof) or to decrease Staufen expression (e.g., by using an antisense of Staufen nucleic acid or of a small interfering RNA of Staufen) in cells thereby preventing or treating a RNA virus infection. Other agents, which decrease the level and/or an activity of Staufen, are also encompassed as agents useful in the treatment of RNA viruses' infection.

In a further embodiment, the present invention relates to the use of small molecules to block Staufen association with Pr55^{Gag} to affect HIV virus assembly, therefore decreasing virus infectivity. Fragment of Staufen domains that interact with Pr55^{Gag} or inversably, the NC domain of Pr55^{Gag} that interact with Staufen may be used to modulate the interaction between the two proteins thereby affecting virus assembly and morphogenesis. IN other words, the present invention also relates to a modulation of the interaction between between Staufen and Pr55^{Gag} in order to modulate RNA virus infection.

In another embodiment, the present invention relates to methods for treating an animal (such as a human) infected with a RNA virus or preventing an RNA virus infection, which comprises an administration thereto of a composition comprising a therapeutically effective amount of a Staufen (such as mammalian Staufen) polypeptide, and/or Staufen nucleic acid molecule encoding same (or vector comprising same), and/or modulators of Staufen activity. In one embodiment, the present invention relates to an administration of a recombinant Staufen molecule having an additional antiviral activity (i.e. RNAse or protease activity).

The invention further relates to the use of polypeptides and nucleic acid molecules encoding same of the present invention to target molecules into virions of RNA viruses. In a particular embodiment, such targeting finds utility for example, in packaging cell lines.

In a further embodiment, the present invention relates to antisense oligonucleotides or siRNA that are able to decrease Staufen expression (e.g., at the mRNA or protein level) in a cell, thereby decreasing genomic RNA encapsidation and infectivity of viruses.

In another embodiment, the present invention relates to methods for treating an animal (such as a human) infected with a RNA virus or preventing an RNA virus infection, which comprises an administration thereto of a composition comprising a therapeutically effective amount of a Staufen siRNA.

In another embodiment, Staufen is used as a carrier for virion targeting and is part of a fusion/chimeric protein.

In accordance with the present invention, there is therefore provided, an isolated mammalian Staufen protein exhibiting homology to mammalian Staufen as well as lower eukaryotic Staufen.

In accordance with the present invention, there is also provided, an isolated nucleic acid molecule comprising a polynucleotide sequence encoding mammalian Staufen.

In accordance with another aspect of the present invention, there is provided, an isolated nucleic acid molecule comprising a polynucleotide sequence which hybridizes under stringent conditions to a polynucleotide sequence encoding mammalian Staufen or to a sequence which is complementary thereto.

In accordance with yet another aspect of the present invention, there is provided a method of constructing a recombinant vector which comprises inserting an isolated nucleic acid molecule (or portion thereof) of mammalian Staufen (or a derivative thereof) into a vector under the control of appropriate regulatory sequences. In addition, the present invention further relates to a recombinant vector harboring an isolated nucleic acid molecule encoding a C. elegans Staufen or fragments or derivatives thereof. In addition, the present invention relates to recombinant vectors harboring an isolated nucleic acid molecule encoding the molecular determinant of a mammalian or lower eukaryotic Staufen, which is responsible for incorporation into RNA virions. In yet a further aspect, the present invention relates to recombinant vectors harboring a siRNA sequence capable of decreasing Staufen expression in a cell. The vectors comprising a Staufen sequence (or fragment thereof), a siRNA or an antisense of the present invention may be used in gene therapy or as vaccines to treat RNA virus infections, more particularly HIV infections.

In a further aspect, the present invention relates to a method for making a recombinant cell comprising introducing thereinto a recombinant vector harboring a Staufen nucleic acid sequence (or portion thereof) of the present invention.

In a particular embodiment, the present invention, relates to an antibody which recognizes specifically a Staufen polypeptide or derivative thereof of the present invention.

The mammalian Staufen polypeptides and nucleic acid molecules of the instant invention have been isolated from human and mouse. Nevertheless, it will be clear to the person of ordinary skill that the present invention should not be so limited. Indeed, using the teachings of the present invention and those of the art, homologues of hStau and mStau can be identified and isolated from other animal species. Non-limiting examples thereof include monkey, mouse, rat, rabbit, and frog. The significant identity between the human and mouse Staufen protein validates this contention.

The conservation of Staufen between mammals and lower eukaryotes (Drosphila and C. elegans) further supports this notion. In addition, it suggests that certain embodiments of the present invention could be carried out using lower eukaryotic Staufen or fragments or derivatives thereof.

The invention further relates to the morphogenesis RNA virions and more particularly of HIV virions and especially to the packaging of RNA genomes into RNA viruses.

In one embodiment, the present invention relates to means to target molecules to RNA virions. In one particular embodiment, the present invention relates to such means to affect the morphogenesis of such RNA virions, thereby reducing infectivity thereof. In one particular embodiment, the present invention relates to a mammalian Staufen protein, which upon incorporation into HIV-1 virions, significantly decreases the infectivity thereof.

The present invention relates to compounds, compositions and methods useful for modulating RNA virus infections by RNA interference using short interfering RNA (siRNA). In one particular embodiment, the RNA virus infection is a HIV infection. In particular, the present invention features siRNA molecules and methods to modulate the expression of Staufen RNA thereby decreasing RNA virus infectivity.

In another embodiment, the present invention relates to antisense oligonucleotides hybridizing to a nucleic acid sequence encoding Staufen protein (SEQ ID NO: 5, SEQ ID NO:1, SEQ ID NO 6, SEQ ID NO:3, SEQ ID NO:7, and SEQ ID NO:10) which decreases Staufen protein cell expression. The present invention further relates to small double stranded RNA molecules (siRNAs) derived from Staufen nucleic acid sequence (SEQ ID NO:5, SEQ ID NO:1, SEQ ID NO:6, SEQ ID NO:3, SEQ ID NO:7, and SEQ ID NO:10) which also decrease Staufen protein cell expression. The present invention also relates to methods utilizing siRNA or antisense RNA targeted to reduce Staufen mRNA and protein expression and therefore significantly decreasing the infectivity of a virion in which Staufen protein has a role in packaging. In a particular embodiment, the virion is an RNA virus. In an even more particular embodiment, the virion is a HIV virion.

In one embodiment, the present invention features a method of reducing Staufen expression in a subject by administering to the subject a dsRNA (e.g., siRNA) or vector producing same in an effective amount to reduce Staufen expression thereby decreasing an RNA virus infection or preventing such infection. The dsRNA can be modified so as to be less susceptible to enzymatic degradation or to facilitate its delivery to a target cell (e.g., a T lymphocyte). RNA interference (i.e., RNAi) toward a targeted DNA segment in a cell can be achieved by administering a double stranded RNA (e.g., siRNA) molecule to the cell, wherein the ribonucleotide sequence of the double stranded RNA molecule corresponds to the ribonucleotide sequence of the targeted DNA segment. In the particular case where the siRNA is chemically modified or contains point mutations, the antisense region of the siRNAs of the present invention is still capable of hybridizing to the ribonucleotide sequence of the targeted gene (e.g., Staufen mRNA) and to trigger RNAi.

In a further embodiment, the present invention features pharmaceutical composition comprising a siRNA of the present invention, which can be chemically modified, in a pharmaceutically acceptable carrier or diluent. In another embodiment, the present invention features a method for treating or preventing a disease or condition in a subject (e.g., RNA viruses infection such as HIV-1 infection), comprising administering to the subject a composition of the invention under conditions suitable for the treatment or prevention of the disease or condition in the subject (e.g., an RNA virus infection, an HIV-1 infection), alone, or in conjunction with one or more therapeutic compounds.

In one embodiment, pharmaceutical compositions of the present invention comprise a specific nucleic acid sequence (e.g., a mammalian Staufen sequence, siRNA, antisense and the like) or fragment thereof in a vector, under the control of appropriate regulatory sequences to target its expression into a specific type of cell (e.g., a T lymphocyte) thereby reducing or preventing viral infection.

The methods of the present invention can be used for subjects with a preexisting viral infection, or subject predisposed to an viral infection. Aditionnally, the methods of the present invention can be used to correct or compensate for cellular or physiological abnormalities involved in conferring susceptibility to viral infections in patients and/or alleviate symptoms of viral infections or as a preventive measure in patients.

### BRIEF DESCRIPTION OF THE DRAWINGS

Having thus generally described the invention, reference will be made to the accompanying drawings, showing by way of illustration only an illustrative embodiment thereof and in which:

“FIG. 1 shows Staufen sequences and alignment thereof. Panel A shows the alignment of the two cDNAs of the human Stauten cDNAs, designated T1 (SEQ ID NO: 1) and T2 (SEQ ID NO: 3) and the translation of the putative protein sequences thereof, starting at amino acid no.-81 and 1, respectively, and presented as the amino acid sequences in SEQ ID NO: 2 and SEQ ID NO: 4, respectively. The numbers refer to the sequence of the short cDNA. The positions of the 4 dsRNA-binding consensus domains (RBD1 to RBD4) and of the tubulin-binding domain (TBD) are indicated between brackets above the sequence. The sequences were deposited in the GenBank database under accession numbers AF061938 and AF061939. Panel B is similar to FIG. 1A but shows the alternative splicing which occurs in the human Staufen gene and gives rise to 4 alternatively spliced transcripts, namely T1, a 3142 bp nucleotide sequence appearing in SEQ ID NO: 5 and encoding from nucleotide 288 to 1775 the protein appearing in SEQ ID NO: 4; T2, a 3217 bp nucleotide sequence discussed above and appearing in SEQ ID NO: 3 and also encoding from nucleotide 363 to 1850 the protein appearing in SEQ ID NO: 4; T3 (designated T1 in FIG. 1A), a 3506 bp nucleotide sequence appearing in SEQ ID NO: 1 and encoding from nucleotide 409 to 2139 the protein appearing in SEQ ID NO: 2; and T4, a 3348 bp nucleotide sequence appearing in SEQ ID NO: 6 and encoding from nucleotide 494 to 1981 the protein appearing in SEQ ID NO: 4. These 4 transcripts therefore give rise to the two proteins as described in FIG. 1A and in the text below. FIG. 1C shows the nucleic acid and predicted amino acid sequence (SEQ ID NO: 8) of mouse Staufen. FIG. 1D shows an alignment of the mouse and human Staufen (SEQ ID NO: 4), highlighting the significant conservation of the protein during evolution. As per FIG. 1A, the 4 dsRNA binding domains (RBD) and tubulin-binding domains are highlighted.

FIG. 1′ shows an alignment between phylogenetically different Staufen proteins of Drosophila (SEQ ID NO: 9), C. elegans (SEQ ID NO: 10) and human (SEQ ID NO: 4). This alignment permits the elaboration of a consensus sequence for Staufen. As shown in FIG. 1B, T1, T2 and T4 give rise to the short protein of 55 kba (SEQ ID NO: 4) while T3 gives rise to the 63 kDa protein (SEQ ID NO: 2). FIG. 1′B shows an alignment between region comprising the human Staufen and tubulin-binding domain (SEQ ID NO: 11) and the human MAPiB microtubule-binding domain (SEQ ID NO: 12).

FIG. 2 shows the characterization of the hStau mRNA and proteins. A) Northern blot analysis of hstau:expression in human tissues. A Human Multiple Tissues Northern Blot (Clontech) was-hybridized with the 1.2 kbp BamHI fragment of hStau cDNA. Lane 1, brain; lane 2, pancreas; lane 3, heart; lane 4, skeletal muscles; lane 5, liver; lane 6, placenta; lane 7, lung; lane 8, kidney). B) Western blot experiment with anti-hStau antibodies. Lane 1, HeLa cell extracts; lane 2, HEK 293 cell extracts. C) HEK cells were transfected with cDNAs coding for either the short (lane 2) or the long (lane 3) hStau isoforms, lysed and analysed by western blotting using the anti-hStau antibodies. Mock-transfected cells are shown in lane 1. D) Schematic representation of the Drosophila (accession number M69111), mammalian and C. elegans (accession number U67949) Staufen proteins. The human protein P1 has an insertion of 81 amino acids at its N-terminal extremity, as compared to protein P2. Large open and black boxes represent the full-length and short dsRNA-binding domains, respectively. Small boxes and lines are regions of high and low sequence similarity, respectively. The hatched boxes indicate the position of the region which is similar to the MAP1B microtubule-binding domain. The percentage of identity between the domains of the human and invertebrate proteins is indicated.

FIG. 3 shows an RNA-binding assay. A) Bacterially expressed his/hStau (lanes S) and his/NEP (lane N) fusion proteins or B) bacterially-expressed MBP/mStau (lanes S) or MBP/aminopeptidase fusion proteins (lane A), were electrophoresed on a polyacrylamide gel, transferred to nitrocellulose, and incubated with [³² P]-labeled nucleic acids, in the presence or absence of cold competitors, as indicated below each gel. After extensive washing, binding was detected by autoradiography.

FIG. 4 shows a tubulin-binding assay. Bacterially expressed MBP/hStau (lanes S) or MBP/aminopeptidase (lanes A) fusion proteins were electrophoresed on SDS-polyacrylamide gels, transferred to nitrocellulose, and incubated with tubulin or actin. After extensive washing, tubulin and actin were detected with monoclonal anti-tubulin or anti-actin antibodies, respectively. As controls, the same experiments were also performed in the absence of either tubulin or anti-tubulin antibodies. Purified actin was also loaded on the gel as control (lane C).

FIG. 5 shows a molecular mapping of the dsRNA- and tubulin-binding domains. Bacterially expressed MBP/mStau (lanes 1), MBP/mStau deletion mutants (lanes 2-7) or MBP/aminopeptidase (lanes C) fusion proteins were electrophoresed on a polyacrylamide gel, transferred to nitrocellulose, and incubated either with [32P]labeled 3′-UTR bicoid RNA (A) or tubulin and anti-tubulin antibodies (B), and revealed as described above. C) Schematic representation of the mutant proteins. Their RNA- and tubulin-binding responses are indicated.

FIG. 6 shows a subcellular localization of the GFP/hStau fusion proteins. COS7 cells were transfected with cDNAs coding for either the hStau/GFP (A, B) or TBD/GFP(C) fusion proteins, or GFP alone (D). Untreated (A, C, D) or Triton X-100 treated (B) cells were fixed and visualized by autofluorescence. Bar=20 mm.

FIG. 7 shows a co-localization of hStau with markers of the rough endoplasmic reticulum (RER) using confocal microscopy. A cDNA coding for an hStau/HA fusion protein was transfected into COS7 cells. Triton X-100 treated cells were fixed and double-labeled with anti-HA (B) and anti-calreticulin (A) or anti-HA (E) and anti-calnexin (D). Anti-HA was detected with Texas Red-coupled anti-mouse IgG antibodies using the Texas Red channel, whereas anti-calreticulin and anti-calnexin were detected with fluorescein-conjugated anti-rabbit IgG antibodies, using the fluorescein channel. C and F are the superposition of A-B and D-E, respectively. No overlap was observed between the fluorescein and Texas Red channels. Bar=10 mm.

FIG. 8(A) shows a Northwestern analysis of hStau TAR RNA-binding. Extracts of bacteria expressing either histidine (his)-tagged hStau (lane 1) or his-NEP (neutral endopeptidase, lane 2) fusion proteins were electrophoresed on a polyacrylamide gel (PAGE), transferred to nitrocellulose and incubated for 2 h with a uniformly [³²P]labelled TAR(1-80) RNA in 50 mM NaCl, 10 mM MgCl₂, 10 mM Hepes, pH 8.0, 0.1 mM EDTA, 1 mM DTT, 0.25% milk. After extensive washing, the membrane was exposed to autoradiographic film. (B). shows a sucrose density gradient analysis of hStau in HIV-1.50×10⁶ cpm of microfiltered and ultracentrifuged virus HxBru was layered onto a continuous 20-60% sucrose gradient, ultracentrifuged at 136 000×g for 16 h. 16-0.7 mL fractions were collected and RT activity was measured by standard assay. Each fraction was subsequently diluted to 20% sucrose and centrifuged at 136000×g for 1 hr to pellet virus particles. After rinsing, the virus pellet was resuspended in PBS and 2× Laemmli loading buffer was added before loading onto a 10% PAGE. The proteins were transferred to nitrocellulose and probed with a rabbit anti-hStau antibody. hStau was visualized using the enhanced chemiluminescence (ECL) kit (Amersham, Mississauga, ON). (C). shows a subtilisin protease resistance assay. Subtilisin assays were performed essentially according to Ott et al., (1995, AIDS Res. Hum. Retroviruses 11, 1003; 1996, J. Virol. 70, 7734) with minor modifications. 70×106 cpm of pelletted virus preparations were treated (+) or mock treated (−) with 1 mg/mL subtilisin (Boehringer Mannheim, Montreal, PQ) in 10 mM Tris-HCl, pH 8, 1 mM CaCl₂, containing 1.5 mg/mL bovine serum albumin (ICN Biochemicals, Montreal, PQ) for 24 h at 37° C. Virus was then pelletted as above and resuspended in PBS, and made to 1×Laemmli and then loaded onto PAGE followed by Western blotting. The blot was sequentially probed with anti-gp120 (Palker et al. (1987) Proc. Natl. Acad. Sci. U.S.A. 84:2479; Palker et al. (1988) Proc. Natl. Acad. Sci. U.S.A. 85:1932; Palker et al. (1989) J. Immunol. 142:3612), a mouse monoclonal antibody #3H11-Cl to p17 (Cogniaux et al. (1990), J. Immunol. Meth. 128:165), a human patient's serum (#162) to reveal p24, and anti-hStau. (D) shows hStau incorporation into virus particles from clinical isolates and the retroviruses HIV-2, MLV, and CasBr. 293T cells were transfected with proviral constructs encoding HIV-1, HIV-2 (ROD), MLV (kindly provided by Dr. Guy Lemay, University of Montreal) and. CasBr retroviruses (Bergeron et al. (1991), J. Virol. 65:7). Virus (passage #2) was also harvested following infection of MT4 cells with two T-tropic viral clinical isolates (T1 & T2; a kind gift from Dr. Mark Wainberg, McGill AIDS Center). 10×106 RT cpm (HIV-1, HIV-2 and MLV) were loaded onto gels and incorporated hStau was assessed by Western blotting. The 55 and 63 kDa hStau species are due to translation initiation from alternatively spliced transcripts (Wickham et al. (1996), Genomics 36:527). Longer exposures reveal both species in all lanes. E. hStau is the only TAR-binding protein to be virion incorporated. Three sets of 25 000 293T cell equivalents (C) and 50 ng p24 virus equivalents (V) were run in parallel on 10% PAGE and each of three blots was probed with antibodies to hStau, TRBP (kindly provided by Dr. Sundararajan Venkatesan, NIAID), and PKR (kindly provided by Dr. Antonis Koromilas, McGill University). For the assessment of Tat in virus particles, 293T cells were transfected with pNL4.3 and at 48 h postinfection (p.i.) cells were lysed in Laemmli buffer and 25 000 cell equivalents were run in parallel with 50 ng p24. An amino-terminal anti-peptide Tat antibody was used for Western blot analysis. Antigens were revealed by ECL and are indicated by bold arrowheads. TRBP, PKR and Tat were undetectable in virion preparations in longer exposures of Western blots.

FIG. 9 shows a localization of hStau in cotransfected 293T cells by confocal laser scanning microscopy. 293T cells were cotransfected with pNL4.3 and a plasmid encoding a HA-tagged hStau (Wickham et al. (1996), supra). 36 h posttransfection, cells were trypsinized and plated on glass slides and allowed to grow for 12 h. After washing, cells were fixed with acetone:methanol (50:50) and allowed to dry. Indirect immunofluorescence was performed using a mouse anti-HA monoclonal (12CA5, Boehringer Mannheim) and a rabbit anti-p24 (Barr et al. (1987), U.C.L.A. Symp. Mol. Cell. Biol. New Ser. 43:205; Steimer et al. (1986), Virology 150:283). Texas Red- and fluorescein-conjugated secondary antibodies were employed to reveal p24 and HA-hStau, respectively. Confocal laser scanning microscopy was performed using a Zeiss LSM410 microscope with excitation wavelengths of 488 nm and 568 nm for fluorescein and Texas Red, respectively. Emission filters for fluorescein and Texas Red were BP515-540, and BP575-640, respectively. p24 (A), hStau (B), and superimposed images (C) are presented. The yellow regions indicate colocalization of p24 and hStau (mostly at the cell periphery). A representative cell is shown.

FIG. 10 shows hStau incorporation correlating with genomic RNA encapsidation in HIV-1 particles. Proviral DNAs [(wildtype, NC (14,15), vpr− or vpr+ (6) and psi mutants (Miele et al. (1996), J. Virol. 70, 944)] were transfected into 293T cells and equal quantities of virus were loaded onto 12% PAGE and probed with anti-hStau (A) and anti-p17 (Figure B; Cogniaux et al., 1990, supra) antisera and antigens were revealed by ECL. In C, RNA was isolated from equal quantities of virus using an NP-40 lysis method (Mouland et al. (1992), Mol. Endocrinol. 6:1781) and probed with a [³²P]-labelled probe to the Gag mRNA leader (Yao et al. (1998), J. Virol. 72:4686-4693). Lane 1, pNL4.3; lane 2, HxBru; lane 3, ²⁸C/9C-S NC; lane 4, ¹⁵C/¹⁸C-S NC; lane 5, ³⁶C/³⁹C—S NC; lane 6 delta ¹⁴K-⁵⁰T NC; lane 7, psi signal mutant; lane 8, HxBru Vpr− provirus; lane 9, HxBru Vpr+ provirus.

FIG. 11 shows overexpression of hStau causing a decrease in infectivity of HIV-1 particles. 10 μg pNL4.3 was transfected into 293T cells with or without an expression plasmid encoding HA-hStau at a 1:1.3 molar basis (or KS DNA carrier). (A), Virus was prepared from mock, pNL4.3 and pNL4.3+hStau transfected cells and used in Western blot analysis using equal quantities of p24 in each lane. For infectivity assays, equal quantities of p24 were used to infect MAGI and BF-24 indicator cells and infectivity was quantitated at 48 h p.i. by calorimetric and CAT activity assays, respectively. (B), BF-24 cells were washed extensively and lysed by freeze-thaw in 0.25 M Tris, pH 7.5, followed by heat inactivation. CAT activity in cells was determined by standard assay by thin layer chromatography (a representative result is shown here). (C), The data shown are the means and standard errors of the means (S.E.M.) from three independent infectivity assays in BF-24 cells. Relative CAT activity (compared to the pNL4.3 lane which is set to 1) was calculated by phosphoimager analysis using the Molecular Dynamics ImageQuant software. MAGI assay results conferred with those from BF-24 assays revealing a 4-fold.(0.3, S.E.M.) reduction in the number of blue □-galactosidase-positve cells 48 h p.i. (7).

FIG. 12 shows one embodiment of primer combinations used in PCR amplifications for Gag constructs based on HIV-1 HxB2 pr55^{Gag}.

FIG. 13 shows one embodiment of the construction and characterization of Staufen-expressing HIV-1 chimeric proviruses. (A) Schematic representation of chimeric proviruses. cDNAs coding for either wild-type Staufen protein, the full-length Staufen with a point mutation in the RNA-binding domain dsRBD3 (StaufenF135A) and a C-terminal truncated mutant containing dsRBD2,3 and 4 (dsRBD2-4) were inserted in the nef open reading frame of pNL4-3/PKR. Staufen proteins were tagged with an HA epitope. (B) Expression of chimeric proviruses. 293T cells were transfected with an empty vector (lane M), pNL4-3 (lane 1), pNL4-3/Staufen-HA (lane 2), pNL4-3/Staufen^{F135A}-HA (lane 3) or pNL4-3/dsRBD2-4-HA (lane 4) and cell extracts were analyzed by Western blotting using anti-HA and anti-CA antibodies to monitor expression of both Staufen-HA and Gag proteins.

FIG. 14 shows that Staufen and pr55 ag cofractionate on sucrose gradients. (A), 293T cells were transfected with pNL4-3 and cell extracts were separated on a 20-60% sucrose gradient. Fifteen fractions were collected and analyzed by Western blotting using mouse monoclonal anti-Staufen, anti-CA and anti-ribosomal protein L7 antibodies as indicated. Input from mock (M) and transfected (T) cells are shown on the right. (B), Same experiment as in (A) except that 293T cells were transfected with pNL4-3/Staufen-HA. Staufen was detected with a mouse monoclonal anti-HA antibody. (C), 293T cells were transfected with pNL4-3/Staufen-HA and lysed in a buffer containing 0.5% NP-40. Cell lysates were separated on a 10-60% sucrose gradient containing 0.5% NP-40. Fractions were analyzed by Western blot analyses using anti-HA, anti-CA and anti-L7 antibodies as indicated.

FIG. 15 shows that treatment of the Staufen-Gag complexes does not affect their interaction. (A) 293T cells were transfected with either an empty vector (M) or pNL4-3 (T) and the cell lysates were immunoprecipitated using anti-CA antibodies. Endogenous Staufen was identified in cellular lysates (A, top-left panel). Following immunoprecipitation using the anti-CA antibody, Staufen (A, top-right panel) and Gag products (A, lower panel) were identified by Western analyses in the immunoprecipitates. (B, C) 293T cells were transfected with an empty vector (lane M), pNL4-3 (lane 1), pNL4-3/Staufen-HA (lane 2), pNL4-3/Staufen^{F135A}-HA (lane 3) or pNL4-3/dsRBD2-4-HA (lane 4) and cell extracts were immunoprecipitated with anti-CA (B) or anti-HA (C) antibodies. Proteins in the pellets were analyzed by Western blotting using anti-HA and anti-CA as indicated. (D) 293T cells were transfected with an empty vector (lane M), pNL4-3 (lane 1) or pNL4-3/Staufen-HA (lane 2). Cell lysates were incubated in the presence (+) or absence (−) of RNases A and T1 during 30 minutes prior to immunoprecipitation analysis. The asterisk (*) shows non-specific IgG labeling. Data shown are representative of four independent experiments.

FIG. 16 shows that Staufen binds pr55^{Gag} via the NC domain. (A) Schematic representation of pr55^{Gag} and deletion mutants fused to Rluc, labelled 1-5. (B, C) 293T cells were cotransfected with these constructs [labeled 1 to 5 in (A) and corresponding to lanes 1 to 5 in (B, C)] and a Staufen-HA expressor. Cell lysates were analyzed by Western blotting using anti-CA and anti-HA antibodies to detect expression of the proteins (B) or immunoprecipitated with anti-CA antibodies (C). Co-immunoprecipitated proteins were analyzed by Western blotting using anti-HA and anti-CA (as control) antibodies. The asterisk (*) shows non-specific IgG labeling. Data shown are representative of two independent experiments.

FIG. 17 shows that Staufen directly binds the HIV-1 NC domain of pr55^{Gag} in living cells. (A) 293T cells were cotransfected with Staufen/YFP and different Rluc-fused truncated Gag expressors. BRET ratios were defined as described in Example 26. N is the replicate number and the error bars represent the SEM calculated from at least three experiments for each interaction set. (B) 293T cells were cotransfected with constant amounts of pCMV-CA-pl/Rluc and varying amounts of either Staufen/YFP or YFP expressors. The graph presented here is a representative example of the saturation curves performed to provide evidence for a specific interaction between the proteins. BRET ratios were plotted as a function of the “excited YFP activity/total Rluc activity” ratio, allowing comparison of BRET ratios between Staufen-YFP and YFP when expressed at the same levels.

FIG. 18 shows the selective association of HIV-1 genomic RNA with Staufen-containing complexes. (A) Schematic representation of the three different primer pair combinations. Each primer pair is specific for the amplification of either the 9 kb genomic RNA (primer pair #1) or the spliced 4 kb (primer pair #2) or 1.8 kb (primer pair #3) RNA species, respectively. (B, C, D) 293T cells were transfected with empty vector (lane 1), pNL4-3 (lane 2), pNL4-3/Staufen-HA (lane 3), pNL4-3/Staufen^{F135A}-HA (lane 4) or pNL4-3/dsRBD2-4-HA (lane 5) and cell lysates were immunoprecipitated with anti-HA antibodies. Coimmunoprecipitated RNAs were RT-PCR amplified with the primer pair described above. PCR products obtained with primer pair #1 (B), #2 (C) and #3 (D) were analyzed on 0.8% agarose gels. Negative controls included RT-PCR amplification in the absence of RNA (lane 6) or reverse transcriptase (lane 7) and amplification of mock-transfected cells (lane 8). Positive controls included RT-PCR amplification of RNA from pNL4-3-transfected cells before immunoprecipitation (lane 9). Amplification of RNA from cell extracts in the absence of reverse transcriptase did not yield a PCR signal (not shown). (E, F) PCR products obtained with primer set #2 (E) or #3 (F) were analyzed on 6% denaturing acrylamide gels after 2 or 5 additional PCR cycles in the presence of [³²P]dCTP. Products from spliced RNA species are indicated on the right.

FIG. 19 shows that siRNA-mediated knock-down of Staufen expression results in decreased HIV-1 infectivity. (A)Western Blot analysis of Staufen and HIV-1 structural proteins levels in 293 T cells 48 h post-transfection. HxBRU plasmid (2 μg) were transfected in the presence of a non silencing (N-S) siRNA (lane 2) and with 50 nM of Staufen siRNA 3084 directed against Staufen (lane 3). Blots were probed with monoclonal antibodies to Staufen (9E9) and anti-GAPDH to control for protein loading. (B) RT-PCR analysis of intracellular Staufen mRNA levels 48 h after co-transfection of HxBRU and siRNA. RNA was purified from a portion of the samples described for A. 1 ug of RNA from each sample was reverse transcribed and amplified with oligonucleotides designed for Staufen and GAPDH to control amount of RNA. Western Blot and RT-PCR quantification of intracellular Staufen protein or mRNA seen in A and B was done with a Can berra Packard Alpha-imager system. The average level of Staufen protein or mRNA present in each sample is given below each lane as a percentage relative to the level found in cells treated with HxBRU and a non-silencing siRNA. Percent standard errors (SE) are given based on the average of three independant experiments. (C) GFP-Based infectivity Assay. The infectivity of virus preparations was determined by infecting CEM-GFP indicator T cells as described in Example 29. Data are expressed as relative infectivity, where HxBRU (treated with a N-S siRNA) infectivity was arbitrarily set to a value of 1. Standard error (SE) is given based on the average of 5 independent experiments.

FIG. 20 shows a proposed model for Staufen involvement in the post-transcriptional steps of HIV-1 lifecycle. Following pr55^{Gag} synthesis on polysomes, pr55^{Gag} interacts with Staufen (Step 1). The Staufen-pr55^{Gag}-HIV-1 genomic RNA complex is released from ribosomes and migrates towards the assembly complexes (Step 2). The Staufen/pr55^{Gag}/genomic RNA ternary complexes are assembled as virion particles at the plasma membrane (Step 3). Following viral protease activation, most of the Staufen is excluded from assembly complexes leading to encapsidation of only 2-10 Staufen molecules per virion (Step 4; Barr et al., 1987, supra, and Steimer et al., 1986, supra). See text for additional discussion of this model.

FIG. 21 shows the association of Staufen and TRBP with HIV-1 Gag during proviral expression. (A) Staufen, TRBP and pr55^{GAG} cofractionate on sucrose gradients. 293T cells were transfected with pNL4-3/Staufen-HA and cell extracts were separated on a 20-60% sucrose gradient. Fisteen fractions were collected and analyzed by western blotting using anti-HA, anti-TRBP, anti-CA and anti-ribosomal protein L7 antibodies as indicated. Input from mock (M) ant transfected cells (T) are shown on the right. Peak fraction no.13 is shown in a box. (B), 293T cells were transfected with pNL4-3 and expressing construct of either Staufen-HA or Myc-TRBP. The resulting cell lysate were immunoprecipitated using anti-CA antibodies. Immunoprecipitated pellets were analyzed by Western blotting with anti-HA, anti-Myc and anti-CA, as indicated.

FIG. 22 shows that Staufen is found in UHC-1 promonocytic cells. (A) 150×108 cpm (RT cpm) (A) or 250×108 cpm (B) of purified virus derived from chronically infected UHC-1 promonocytic cells (a cellular clone derived from HIV-1 strain HIV-IIIB chronically infected U-937 promonocytic cells) were fractionated on a 20-60% sucrose gradient. 15-16 fractions were collected and an aliquot from each fraction was loaded on SDS-PAGE and blotted for Staufen (upper panels in A and B; rabbit antibody described in ref. 34) or p24 (lower panels in A and B). The virus in B was treated with NP-40 to completely lyse virions prior to loading on sucrose gradients. Staufen is shown to cosediment with p24 in the absence and presence of detergent. * Nonspecific band.

FIG. 23 shows that TAR RNA-Binding Protein (TRBP) associates with all three HIV-1 RNA species. 293T cells were co-transfected with pNL4-3 and Myc-TRBP or Staufen-HA expressors. HIV-1, TRBP and Staufen expression levels were checked by Western blot analyses (see additional figure: Reviewer 2: FIG. R2-B of the co-immunoprecipitation of Gag and TRBP). Cell lysates were immunoprecipitated with mouse monoclonal anti-Myc (ascites #9E10) antibody. Coimmunoprecipitated RNAs were treated with DNase I, purified by Trizol extraction and amplified by RT-PCR with the primer pairs described in FIG. 6A. PCR products obtained from IP anti-Myc with primer pairs #1 (HIV-1 genomic RNA), #2 (HIV-14 kb RNA species) and #3 (HIV-1 1.8 kb RNA species) were analyzed on 1% agarose gels and are shown in lane 2 (pNL4-3+Myc-TRBP). Negative controls included RT-PCR amplification in the absence of reverse transcriptase (lane 3), RNA (lane 7), cDNA (lane 8) and RT-PCR after anti-Myc IP on HIV-1 and Staufen-HA positive cell lysate (lane 1). Positive controls included PCR on pNL4-3 (lane 9) and RT-PCR amplification of RNA from pNL4-3-transfected cell lysates (lane 5). Amplification of these cell extracts in the absence of reverse transcriptase (lane 6) and amplification of mock-transfected cells (lane 4) are also shown. *non-specific amplification in mock.

Other objects, advantages and features of the present invention will become more apparent upon reading of the following non-restrictive description of preferred embodiments with reference to the accompanying drawings, which are exemplary and should not be interpreted as limiting the scope of the present invention.

### DESCRIPTION OF ILLUSTRATIVE EMBODIMENT

The present invention therefore relates to Staufen, a double-stranded RNA-binding protein which binds dsRNA via each of two full-length dsRNA-binding domains and tubulin via a region similar to the microtubule-binding domain of MAP1B. Immunofluorescent double-labeling of transfected mammalian cells revealed that Stau is localized to the rough endoplasmic reticulum (RER), implicating this RNA-binding protein in mRNA targeting to the RER. These results are the first demonstration of the association of an RNA-binding protein with the RER, implicating this class of proteins in the transport of RNA to its site of translation.

The human homologue of the double-stranded RNA (dsRNA)-binding protein, Staufen, is shown herein to be incorporated into HIV-1 virions, and this correlates with HIV-1 genomic RNA encapsidation. hStau is incorporated into clinical isolates of HIV-1, and several other retroviruses including HIV-2 and murine leukemia virus, and non-retroviral RNA viruses such as Reovirus, but is not detectable in DNA viruses. When hStau is overexpressed, a corresponding increase of hStau in virions is observed. Strikingly, however, this increase in hStau incorporation into HIV-1 is accompanied by a dramatic impairment of HIV-1 infectivity. Moreover, knockdown of Staufen expression by small interfering RNA (siRNA) in HIV-1 expressing cells also demonstrates that this cellular protein is important for the generation of infectious virus. This is the first demonstration of a dsRNA-binding protein within HIV-1 particles. These novel and unexpected findings have important implications not only in retroviral genome sorting, assembly and infectivity, but also in RNA virus therapy in general and more in particularly HIV-1 therapy. Therefore, modulation of Staufen expression by gene therapy, vaccine therapy, or by the use of small molecules interfering with Staufen-Gag interactions may be used in accordance with the present invention to treat or prevent RNA virus infections, more particularly HIV infections.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.) or The Harper Collins Dictionary of Biology (Hale & Marham, 1991, Harper Perennial, New York, N.Y.), Rieger et al., Glossary of genetics: Classical and molecular, 5^{th} edition, Springer-Verlag, New-York, 1991; Alberts et al., Molecular Biology of the Cell, 4^{th} edition, Garland science, New-York, 2002; and, Lewin, Genes VII, Oxford University Press, New-York, 2000. Generally, the procedures of molecular biology methods and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. (1994, Current Protocols in Molecular Biology, John Wiley & Sons, New-York).

In the present description, a number of terms are extensively utilized. In order to provide a clear and consistent understanding of the specification and claims, including the scope to be given such terms, the following definitions are provided

Definitions

As used herein, the terms “nucleic acid molecule” or “polynucleotides”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11 ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

The terminology “Staufen nucleic acid” or “Staufen polynucleotide” refers to a native Staufen-nucleic acid sequence. In one embodiment, the Staufen nucleic acid sequence comprises the sequence or part thereof of anyone of the sequences set forth in SEQ ID NO 5, SEQ ID NO:3, SEQ ID NO1, SEQ ID NO:6, and SEQ ID NO:7. In one particular embodiment, the Staufen nucleic acid encodes a Staufen protein such as one having the sequence of SEQ ID NO 4, SEQ-ID NO:2, SEQ ID NO:8, SEQ ID NO:10).

The term “isolated nucleic acid molecule” refers to a nucleic acid molecule purified from its natural environment. Non-limiting examples of an isolated nucleic acid molecule is a DNA sequence inserted into a vector, and a partially purified polynucleotide sequence in solution.

The term “recombinant DNA” as known in the art refers to a DNA molecule resulting from the joining of DNA segments. This is often referred to as genetic engineering.

The term “DNA segment” is used herein, to refer to a DNA molecule comprising a linear stretch or sequence of nucleotides. This sequence when read in accordance with the genetic code (e.g., an open reading frame or ORF), can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions. For example, homologs of human or mouse Staufen could be isolated using an amplification method such as PCR with an amplification pair designed by comparing the homology of the human and mouse sequences.

“Amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA). Replicase-mediated amplification uses self-replicating RNA molecules, and a replicase such as Qβ-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195,4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification is used in the present invention. Those skilled in the art will understand that the oligonucleotide primer sequences of the present invention may be readily used in any in vitro amplification method based on primer extension by a polymerase. (see generally Kwoh et al. (1990), Am. Biotechnol. Lab. 8:14-25; Kwoh et al. (1989), Proc. Natl. Acad. Sci. USA 86:1173-1177; Lizardi et al. (1988), BioTechnology 6:1197-1202; Malek et al. (1994), Methods Mol. Biol., 28:253-260; and Sambrook et al. (2000), Molecular Cloning—A Laboratory Manual, Third Edition, CSH Laboratories). As commonly known in the art, the oligos are designed to bind to a complementary sequence under selected conditions.

The nucleic acid (i.e. DNA or RNA) for practicing the present invention may be obtained according to well known methods.

As used herein, the term “physiologically relevant” is meant to describe interactions which can modulate a function wich is physiologically relevant. The present invention encompassed for example the transcription of a gene in its natural setting. Of course a binding of a protein (e.g., pr55^{Gag}) or RNA (e.g., HIV-1 RNA) is also considered as a physiologically relevant function since this binding occur in a natural setting

Oligonucleotide probes or primers of the present invention may be of any suitable length, depending on the particular assay format and the particular needs and targeted genomes employed. In general, the oligonucleotide probes and primers of the present invention comprise at least 10 contiguous nucleotides (preferably, 10, 11, 12, 13, 14, 15, 16, 17, 18, 19, 20, 21, 22, 23, 24, 25, 26, 27, 28, 29, 30, 31, 32) of a Staufen nucleic acid molecule or its complementary sequence. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 15 and 30 nucleotides in length are usually designed and used in the art. As well known in the art, probes ranging from 20 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention the oligonucleotide probes or primers are adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 200, Molecular Cloning—A Laboratory Manual, 2nd Edition, CSH Laboratories; Ausubel et al., 1989, in Current Protocols in Molecular Biology, John Wiley & Sons Inc., N.Y.) defines an oligonucleotide which is capable of annealing to a target sequence, thereby creating a double stranded region which can serve as an initiation point for nucleic acid synthesis under suitable conditions. Primers can be, for example, designed to be specific for certain alleles so as to be used in an allele-specific amplification system.

The terms “DNA oligonucleotide”, or “DNA molecule” or “DNA sequence” refer to a molecule comprised of the deoxyribonucleotides adenine (A), guanine (G), thymine (T) and/or cytosine (C). Oligonucleotide or DNA can be found in linear DNA molecules or fragments, viruses, plasmids, vectors, chromosomes or synthetically derived DNA.

By “RNA” or “mRNA” is meant a molecule comprising at least one ribonucleotide residue. By ribonucleotide is meant a nucleotide with a hydroxyl group at the 2′ position of a □-D-ribo-furanose moiety. The term include double stranded RNA, single stranded RNA, isolated RNA such as partially purified RNA, essentially purified RNA, synthetic RNA, recombinantly produced RNA, as well as altered RNA that differs from naturally occurring RNA by the addition, deletion, substitution and/or alteration of one or more nucleotide. Such alterations can include addition of non-nucleotide material, such as to the end(s) of an siRNA or internally, for example at one or more nucleotides of the RNA molecule. Nucleotides in the RNA molecules of the instant invention can also comprise non-standard nucleotides or chemically synthesized nucleotides or deoxynucleotides. These altered RNAs can be referred to as analogs or analogs of naturally occurring RNA.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra) and are commonly known in the art. In the case of a hybridization to a nitrocellulose filter, as for example in the well known Southern blotting procedure, a nitrocellulose filter (or other such support like nylon) can be incubated overnight at 65° C. with a labeled probe in a solution containing high salt (5×SSC or 5×SSPE), 5× Denhardt's solution, 1% SDS, and 100 μg/ml denatured carried DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° (moderate stringency) or 65° C. (high stringency). The salt and SDS concentration of the washing solutions may also be adjusted to accommodate for the desired stringency. The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 2000, supra). As well known in the art other stringent hybridization conditions can be used (i.e. 42° C. in the presence of 50% of formamide). Other protocols or commercially available hybridization kits (e.g., ExpressHyb™ from BD Biosciences Clonetech) using different annealing and washing solutions can also be used as well known in the art.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e, resulting from a probe hybridizing directly to the target or amplified sequence) or indirect (i.e., resulting from a probe hybridizing to an intermediate molecular structure that links the probe to the target or amplified sequence). A probe's “arget” generally refers to a sequence within an amplified nucleic acid sequence (i.e, a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988 (Ann. Reports Med. Chem. 23:295) and Moran et al., 1987 (Nucl. Acids Res., 14:5019). Probes of the invention can be constructed of either ribonucleic acid (RNA) or deoxyribonucleic acid (DNA), and preferably of DNA.

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. Appropriate hybridization conditions are well known to those skilled in the art, can be predicted readily based on sequence composition and conditions, or can be determined empirically by using routine testing (see Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7.37-7.57, 9.47-9.51 and 11.47-11.57, particularly at §§ 9.50-9.51, 11.12-11.13, 11.45-11.47 and 11.55-11.57).

The terminology “about” as used herein is meant to designate a possible variation of up to 10%. Therefore, a variation of 1, 2, 3, 4, 5, 6, 7, 8, 9 and 10% of a value is included in the term about.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation.

A “label” refers to a molecular moiety or compound that can be detected or can lead to a detectable signal. A label is joined, directly or indirectly, to a nucleic acid probe or the nucleic acid to be detected (e.g., an amplified sequence). Direct labeling can occur through bonds or interactions that link the label to the nucleic acid (e.g., covalent bonds or non-covalent interactions), whereas indirect labeling can occur through a use of a “linker” or bridging moiety, such as additional oligonucleotide(s), which is either directly or indirectly labeled. Bridging moieties may amplify a detectable signal. Labels can include any detectable moiety (e.g., a radionucleotide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Probes can be labeled according to numerous well known methods (Sambrook et al., 2000, supra). It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma [³²P] ATP and polynucleotide kinase, using the Klenow fragment of Pol I of E. coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well known methods.

As used herein, a “primer” defines an oligonucleotide which is capable of annealing to a target sequence, thereby creating a double stranded region which can serve as an initiation point for DNA synthesis under suitable conditions. Primers can be, for example, designed to be specific for certain alleles so as to be used in an allele-specific amplification system. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Of course the design of a primer from a known nucleic acid sequence is well known in the art. As for the oligos, it can comprise a number of types of different nucleotides.

Amplification of a selected, or target, nucleic acid sequence may be carried out by a number of suitable methods. See generally Kwoh et al., 1990, supra. Numerous amplification techniques have been described and can be readily adapted to suit particular needs of a person of ordinary skill. Non-limiting examples of amplification techniques include polymerase chain reaction (PCR), ligase chain reaction (LCR), strand, displacement amplification (SDA), transcription-based amplification, the Qβ replicase system and NASBA (Kwoh et al., 1989, supra; Lizardi et al., 1988, supra; Malek et al., 1994, supra; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patents are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss (1991), Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696.

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will readily recognized by the person of ordinary skill, that the nucleic acid sequence of the present invention can be incorporated into anyone of numerous established kit formats which are well known in the art.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a structural gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often refered to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites. Typically, expression vectors are prokaryote specific or eukaryote specific although shuttle vectors are also widely available.

Prokaryotic expression are useful for the preparation of large quantities of the protein encoded by the DNA sequence of interest. This protein can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine-Dalgarno sequences in addition to the −10 and −35 consensus sequences.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “junctional fragments”, “dunctional segments”, “functional variants”, “functional analogs” or “functional chemical derivatives” of the subject matter of the present invention. “Fragments” of the nucleic acid molecules according to the present invention refer to such molecules having at least 12 nt, more particularly at least 18 nt, and even more preferably at least 24 nt which have utility as diagnostic probes and/or primers. It will become apparent to the person of ordinary skill that larger fragments of 100 nt, 1000 nt, 2000 nt and more also find utility in accordance with the present invention.

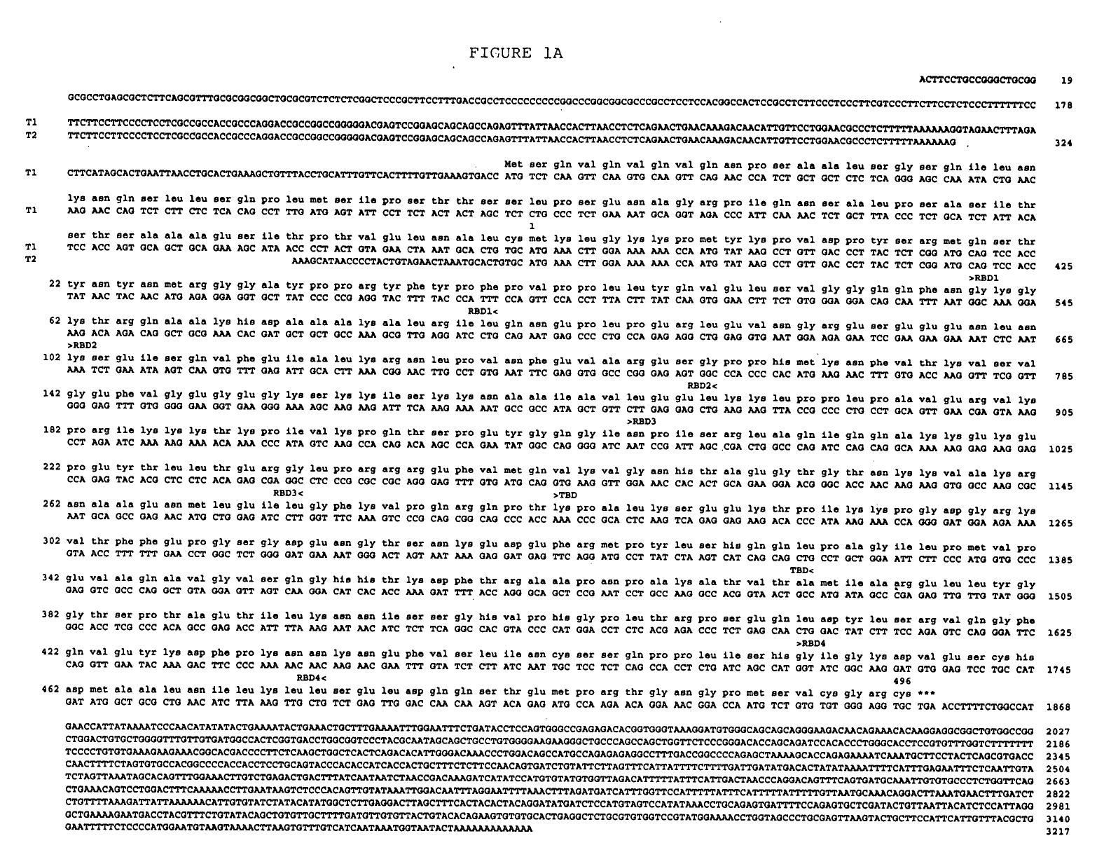

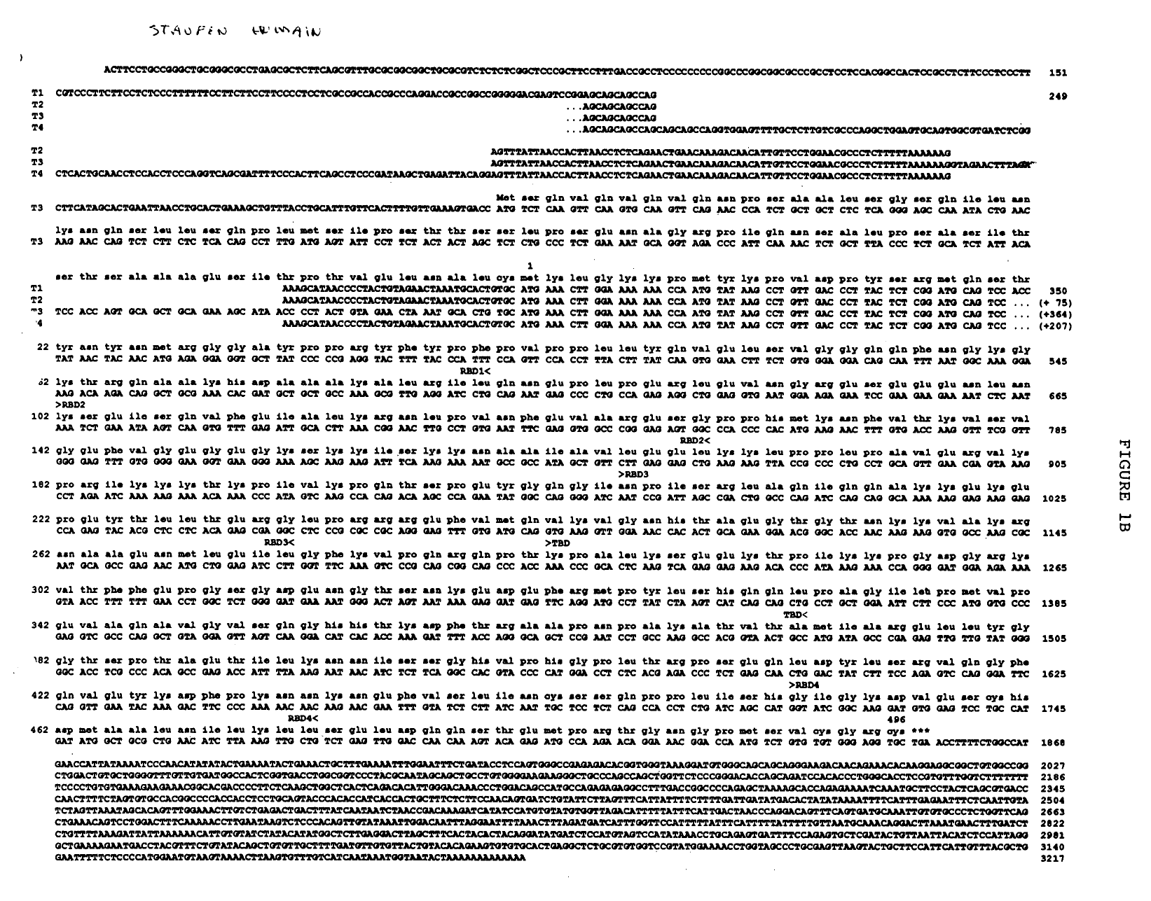

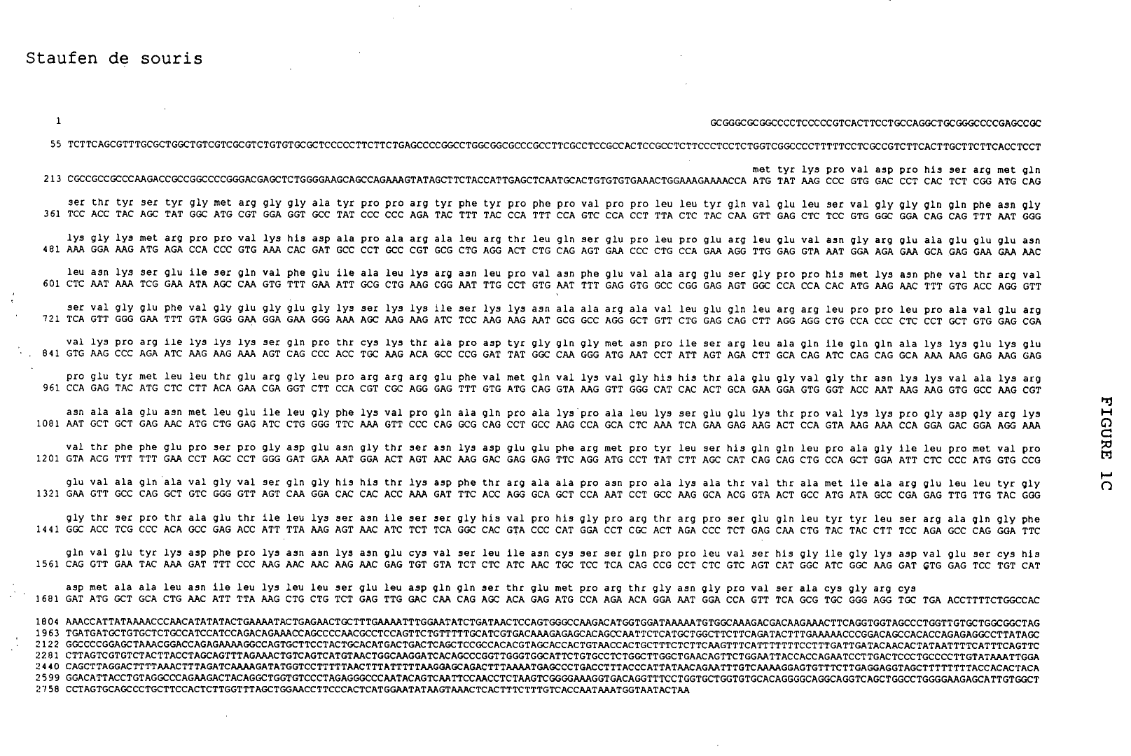

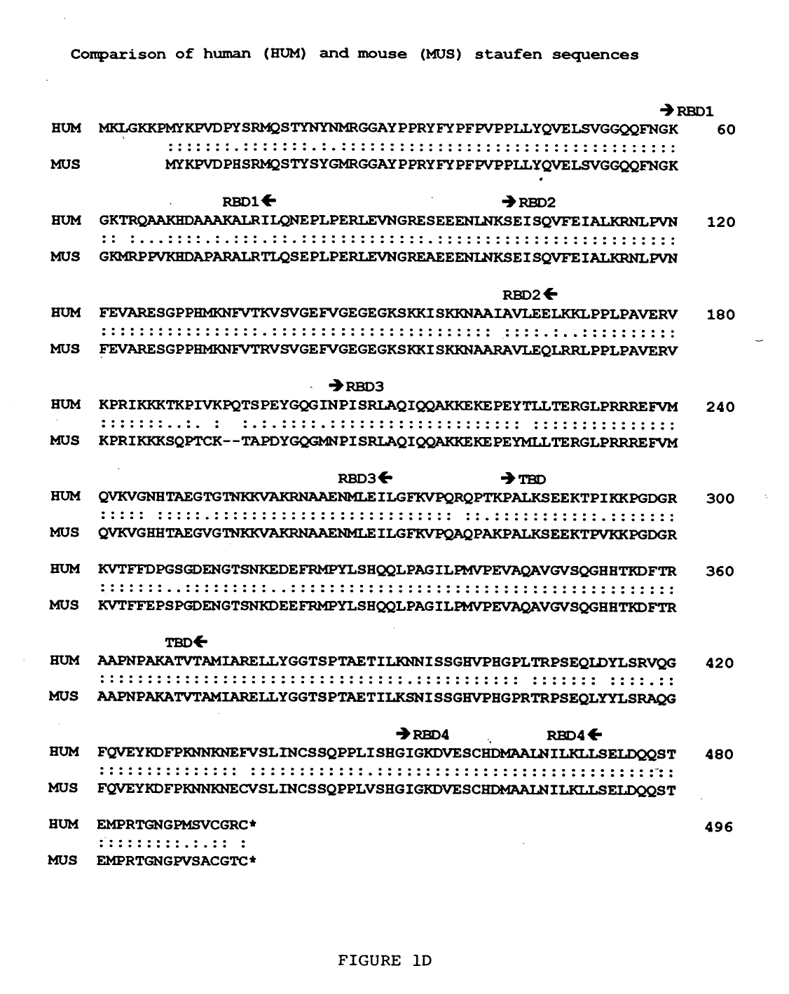

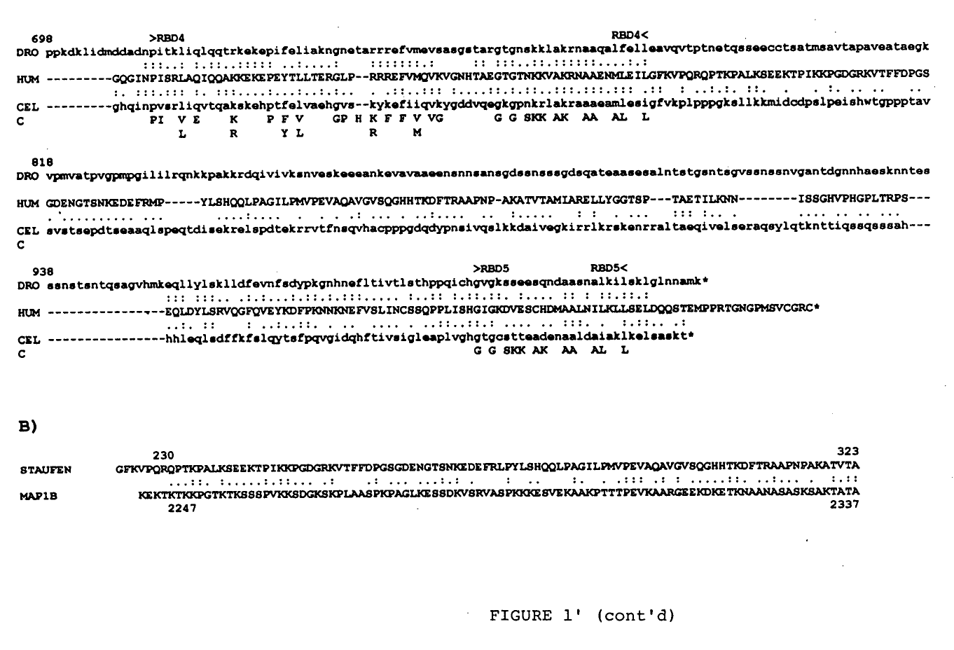

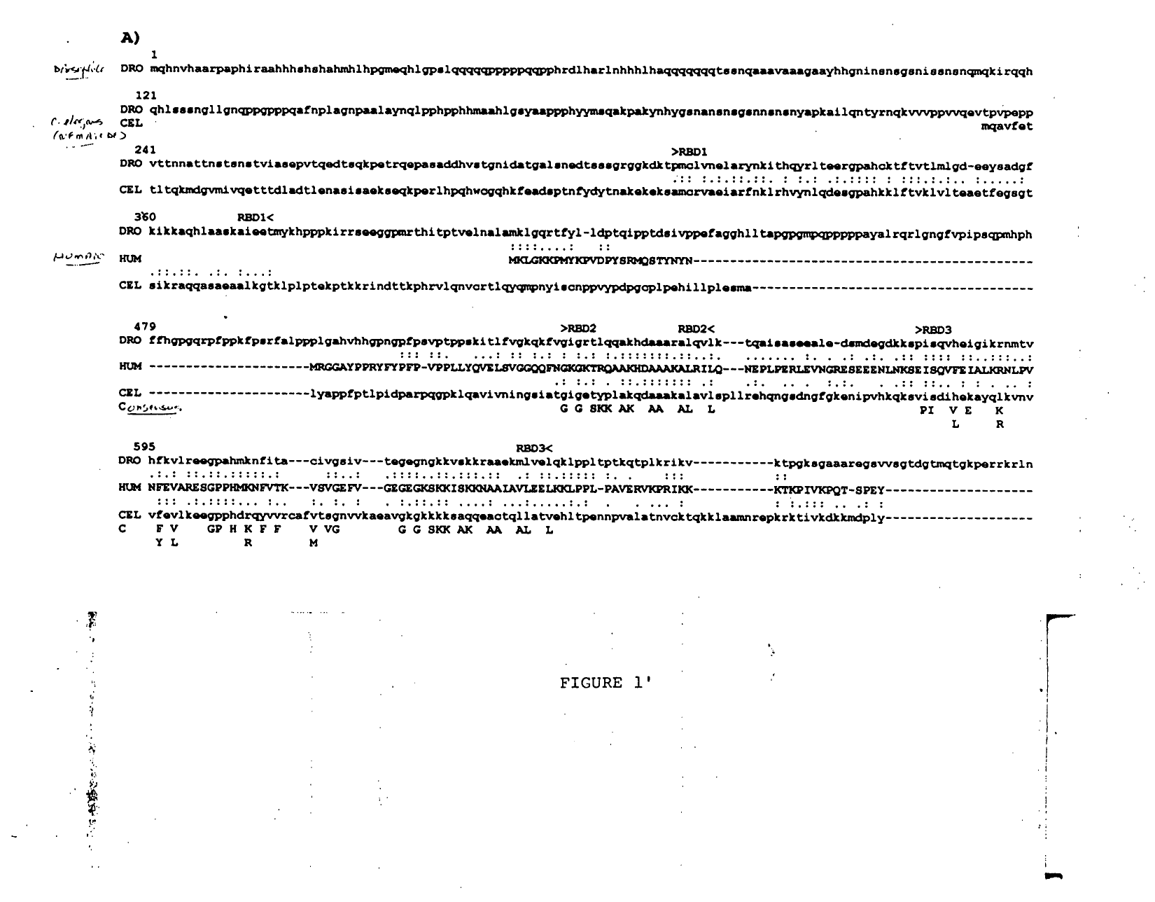

The term “at least 24 nt” is meant to refer to 24 contiguous nucleotides of a chosen sequence such as shown for example in FIG. 1A, 1B, 1C, 1D and 1′.

The term “functional variant” refers herein to a protein or nucleic acid molecule which is substantially similar in structure and biological activity to the protein or nucleic acid of the present invention. For example, a functional variant of Staufen protein would be any variant that retains at least one of Staufen biological activity such as RNA binding activity, tubulin binding activity, Pr55^{Gag} binding activity or HIV-1 genomic RNA binding activity. Similarly, a functional variant of a Staufen nucleic acid is a nucleic acid that when translated into a protein retains at least one of Staufen.s biological activity.

The functional derivatives of the present invention can be synthesized chemically or produced through recombinant DNA technology, all these methods are well known in the art.

The term “molecule” is used herein in a broad sense and is intended to include natural molecules, synthetic molecules, and mixture of natural and synthetic molecules. The term “molecule” is also meant to cover a mixture of more than one molecule such as for example pools or libraries of molecules. Non-limiting examples of molecules include chemicals, biological macromolecules, cell extracts and the like.

The term “compound” is used herein interchangeably with molecule and is similarly defined.

Nucleic acid fragments in accordance with the present invention include epitope-encoding portions of the polypeptides of the invention. Such portions can be identified by the person of ordinary skill using the nucleic acid sequences of the present invention in accordance with well known methods. Such epitopes are useful in raising antibodies that are specific to the polypeptides of the present invention. The invention also provides nucleic acid molecules which comprise polynucleotide sequences capable of hybridizing under stringent conditions to the polynucleotide sequences of the present invention or to portions thereof.

The term hybridizing to a “portion of a polynucleotide sequence” refers to a polynucleotide which hybridizes to at least 10 nt (e.g., 10, 11, 12, 13, 14, 15, 16, 17, 18, 19, 20 . . . ), more preferably at least 18 nt (e.g, 18, 19, 20, 21, 22, 23, 24, 25, 26 . . . ), even more preferably at least 24 nt (e.g., 24, 25, 26, 27, 28, 29, 30 . . . ) and especially to about 50 (e.g, 50, 51, 52, 53 . . . ) nt of a polynucleotide sequence of the present invention.

The present invention further provides isolated nucleic acid molecules comprising a polynucleotide sequences which is at least 95% identical (e.g., 95, 96, 97, 98, 99, 100%), and preferably from 96% to 99% (e.g., 96, 97, 98, 99, 100%) identical to the polynucleic acid sequence encoding the full length Staufen polypeptides (i.e. 55 and 63 kDa hStau) or fragments and/or derivatives thereof. Methods to compare sequences and determine their homology/identity are well known in the art and exemplified herein.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington: The Science and Practice of Pharmacy by Alfonso R. Gennaro, 2003, 21 h edition, Mack Publishing Company. Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, “RNA viruses” is used broadly to cover retroviruses and non-retroviruses (such as Reovirus and poliovirus).

As used herein, HIV is used loosely to refer to HIV-1, HIV-2 and to SIV and related viruses.

The term “subject” or “patient” as used herein refers to an animal, preferably a mammal, most preferably a human who is the object of treatment, observation or experiment.

A functional activity of a polypeptide or protein is any activity associated with a structural, biochemical or physiological activity of the protein (either structural or functiorial). For example, one non-limiting example of a functional activity of Staufen protein includes its RNA binding activity (e.g., HIV RNA) and its intecraction with other proteins (e.g., pr55^{Gag}).

As used herein, the term “purified” refers to a molecule (e.g. nucleic acid) having been separated from a component of the composition in which it was originally present. Thus, for example, a “purified nucleic acid” has been purified to a level not found in nature. A “substantially pure” molecule is a molecule that is lacking in most other components. (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. For the sake of brevity, the units (e.g. 66, 67 . . . 81, 82, . . . 91, 92% . . . ) have not been specifically recited but are considered nevertheless within the scope of the present invention.

The term “isolated polypeptide” refers to a polypeptide removed from its natural environment. Non-limiting examples of isolated polypeptides include a polypeptide produced recombinantly in a host cell and partially or substantially purified polypeptides from such host cells. The polypeptides of the present invention comprise polypeptides encoded by the nucleic acid molecules of the present invention, as shown for example in FIG. 1A, 1B, 1C, 1D and 1′. The present invention also provides polypeptides comprising amino acids sequences which are at least 95% (e.g., 95, 96, 97, 98, 99, 100%) homologous, preferably from 96-99% (e.g., 96, 97, 98, 99, 100%) homologous, even more preferably at least 95% (e.g., 95, 96, 97, 98, 99, 100%) identical and especially preferably from 96% to 99% identical to the full length Staufen polypeptide sequence or fragments or derivatives thereof.

As used herein, the terms “molecule”, “compound” or “ligand” are used interchangeably and broadly to refer to natural, synthetic or semi-synthetic molecules or compounds. The term “molecule” therefore denotes for examples chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeastasis of the cell and/or tissue is compromised by a defect in in modulating gene expression and/or translation. Alternatively, the molecules identified in accordance with the teachings of the present invention find utility in the development of more efficient cell lines or cell extracts for translating mRNAs. Non-limiting examples of diseases and/or conditions in which the protein and/or nucleic acid molecules of the present inventiori find utility include cancer, apoptosis and aberrant proliferation of cells.

As used herein, agonists and antagonists of translation activity also include potentiators of known compounds with such agonist or antagonist properties. In one embodiment, agonists can be detected by contacting the indicator cell with a compound or mixture or library of molecules, for a fixed period of time, and then determining the effect of the compound on the cell.

The level of gene expression of the reporter gene (e.g. the level of luciferase, or β-gal, produced) within the treated cells can be compared to that of the reporter gene in the absence of the molecule(s). The difference between the levels of gene expression indicates whether the molecule(s) of interest agonizes the aforementioned interaction. The magnitude of the level of reporter gene product expressed (treated vs. untreated cells) provides a relative indication of the strength of that molecule(s) as an agonist. The same type of approach can also be used in the presence of an antagonist(s).

Alternatively, an indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules vs in the presence thereof. Of course, the antagonistic effect of a molecule can also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells can be prepared and used in one of the aforementioned “in vitro” tests (such as binding assays or in vitro translations).

As used herein the recitation “indicator cells” refers to cells wherein an interaction between Staufen and dsRNA and/or Staufen and tubulin for example is coupled to an identifiable or selectable phenotype or characteristic such that it provides an assessment of the interaction between these domains. Such indicator cells can be used in the screening assays of the present invention. In certain embodiments, the indicator cells have been engineered so as to express a chosen derivative, fragment, homolog, or mutant of Staufen. The cells can be yeast cells or higher eukaryotic cells such as mammalian cells (WO 96/41169). In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the a Staufen domain with a binding partner (i.e. tubulin). Such an indicator cell could be used to rapidly screen at high-throughput a vast array of test molecules. In a particular embodiment, the reporter gene is luciferase or β-Gal.

As exemplified herein below in one embodiment, at least one Staufen domain may be provided as a fusion/chimeric protein. The design of constructs therefor and the expression and production of fusion proteins are exemplified herein (i.e. Example 2) and are well known in the art (Sambrook et al., 2000, supra; and Ausubel et al., 1994, supra). In a particular embodiment, both the binding partner of Staufen and Staufen are part of fusion proteins.

Non-limiting examples of such fusion proteins include a hemaglutinin A (HA) fusions and Gluthione-S-transferase (GST) fusions, HIS fusions, FLAG fusions, and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non-limiting examples of proteins containing signal sequences. In certain embodiments, it might also be beneficial to introduce a linker (commonly known) between the interaction domain and the heterologous polypeptide portion. Such fusion protein finds utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of Staufen activity.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on an episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 2000, supra; Ausubel et al., 1994, supra). It will be understood that extracts from animal cells or mammalian cells for example could be used in certain embodiments, to compensate for the lack of certain factors in lower eukaryotic indicator cells.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

The present invention further relates to RNA interference (RNAi) to decrease Staufen expression in target cells. “RNA interference” refers to the process of sequence specific suppression of gene expression mediated by small interfering RNA (siRNA) without generalized suppression of protein synthesis. While the invention is not limited to a particular mode of action, RNAi may involve degradation of messenger RNA (e.g., Staufen mRNA) by an RNA induced silencing complex (RISC), preventing translation of the transcribed targeted mRNA. Alternatively, it may involve methylation of genomic DNA, which shuts down transcription of a targeted gene. The suppression of gene expression caused by RNAi may be transient or it may be more stable, even permanent.

RNA interference is triggered by the presence of short interfering RNAs of about 20-25 nucleotides in length which comprise about 19 base pair duplexes. These siRNAs can be of synthetic origin or they can be derived from a ribonuclease III activity (e.g., dicer ribonuclease) found in cells. The RNAi response also features an endonuclease complex containing siRNA, commonly referred to as an RNA-induced silencing complex (RISC), wich mediates the cleavage of single standed RNA having a sequence complementary to the antisense region of the siRNA duplex. Cleavage of the target RNA (e.g., Staufen mRNA) takes place in the middle of the region complementary to the antisense strand of the siRNA duplex (Elbashir et al., 2001, Genes Dev., 15:188).

“Small interfering RNA” of the present invention refers to any nucleic acid molecule capable of mediating RNA interference “RNAi” or gene silencing (see for example, Bass, 2001, Nature, 411:428-429; Elbashir et al., 2001, Nature, 411:494-498; Kreutzer et al., International PCT publication No. WO 00/44895; Zernicka-Goetz et al., International PCT publication No. WO 01/36646; Fire, International PCT publication No. WO99/32619; Mello and Fire, International PCT publication No. WO01/29058; Deschamps-Depaillette, International PCT publication No. WO99/07409; Han et al., International PCT puplication No. WO 2004/011647; Tuschl et al., International PCT publication No. WO 02/44321; and Li et al., International PCT publication No. WO 00/44914). For example, siRNA of the present invention are double stranded RNA molecules from about ten to about 30 nucleotides long that are named for their ability to specifically interfere with protein expression. In one embodiment, siRNA of the present invention are 12-28 nucleotides long, more preferably 15-25 nucleotides long, even more preferably 19-23 nucleotides long and most preferably 21-23 nucleotides long. Therefore preferred siRNA of the present invention are 12, 13, 14, 15, 16, 17, 18, 19, 20, 21, 22, 23, 24, 25, 26, 27, 28 nucleotides in length. Has used herein, siRNA molecules need not to be limited to those molecules containing only RNA, but further encompasses chemically modified nucleotides and non-nucleotides.

The length of one strand designates the length of an siRNA molecule. For example, an siRNA that is described as a 23 ribonucleotides long (a 23 mer) could comprise two opposite strands of RNA that anneal together for 21 contiguous base pairing. The two remaining ribonucleotides on each strand would form what is called an “overhang”. In a particular embodiment, the siRNA of the present invention contains two strand of different lengths. In this case, the longer strand designates the length of the siRNA. For example, a dsRNA containing one strand that is 20 nucleotides long and a second strand that is 19 nucleotides long, is considered a 20 mer.

siRNAs that comprises an overhang are desirable. The overhang may be at the 3′ or 5′ end. Preferably, the overhangs are at the 3′ end of an RNA strand. The length of an overhang may vary but preferably is about 1 to 5 nucleotides long. Generally, 21 nucleotides siRNA with two nucleotides 3′-overhang are the most active siRNAs.

siRNA of the present invention are designed to decrease Staufen expression in a target cell by RNA interference. siRNA of the present invention comprise a sense region and an antisense region wherein the antisense region comprises a sequence complementary to a Staufen mRNA sequence (e.g., SEQ ID NO: 5, SEQ ID NO: 1, SEQ ID NO: 7, SEQ ID NO: 3, SEQ ID NO: 10, and SEQ ID NO: 6) and the sense region comprises a sequence complementary to the antisense sequence of Staufen mRNA. A siRNA molecule can be assembled from two nucleic acid fragments wherein one fragment comprises the sense region and the second fragment comprises the antisense region of siRNA molecule. The sense region and antisense region can also be covalently connected via a linker molecule. The linker molecule can be a polynucleotide linker or a non polynucleotide linker.

In one embodiment, the present invention features a siRNA molecule having RNAi activity against Staufen RNA, wherein the siRNA molecule comprises a sequence complementary to any RNA having a Staufen encoding sequence. A siRNA molecule of the present invention can comprise any contigous Staufen sequence (e.g., 19-23 contiguous nucleotides present in a Staufen sequence such as SEQ ID NO: 5, SEQ ID NO: 1, SEQ ID NO: 7, SEQ ID NO: 3, SEQ ID NO: 10, and SEQ ID NO: 6). In the particular case where alternate splicing produces a family of transcripts that are distinguished by specific exons, the present invention can be used to inhibit gene expression of a particular gene family member through the targeting of the appropriate exon(s) (e.g., to specifically knock down the expression of the 55 kDa or the 63 kDa Staufen protein).

siRNA of the present invention comprises a ribonucleotide sequence that is at least 80% identical to a Staufen ribonucleotide sequence. Preferably, the siRNA molecule is at least 90%, at least 95% (e.g., 95, 96, 97, 99, 99, 100%), at least 98% (e.g., 98, 99,100%) or at least 99% (e.g., 99,100%) identical to the ribonucleotide sequence of the target gene (e.g., Staufen RNA). siRNA molecule with insertion, deletions, or single point mutations relative to the target may also be effective. Mutations that are not in the center of the siRNA molecule are more tolerated. Tools to assist siRNA design are well known in the art and readily available to the public. For example, computer-based siRNA design tool is available on the internet at www.dharmacon.com or are available in the web site of several companies that offers the synthesis of siRNA molecules.

By “complementary” or “complementarity” is meant that nucleic acid can form hydrogen bond(s) with another nucleic acid sequence by either traditional Watson crick base pairing or other non-traditional types of interactions. In reference to the nucleic acid molecules of the present invention, the binding free energy for a nucleic acid molecule with its complementary sequence is sufficient to allow the relevant function of the nucleic acid to proceed (e.g., RNAi activity). For example, the degree of complementarity between the sense and antisense region (or strand) of the siRNA construct can be the same or can be different from the degree of complementarity between the antisense region of the siRNA and the target RNA sequence (e.g., Staufen RNA sequence). Complementarity to the target sequence of less than 100% in the antisense strand of the siRNA duplex (including deleltions, insertions and point mutations) is reported to be tolerated when these differences are located between the 5′-end and the middle of the antisense siRNA (Elbashir et al.,2001, Embo, 20(23):68-77-6888). Determination of binding free energies for nucleic acid molecules is well known in the art (e.g., see Turner et al., 1987, J. Am. Chem. Soc. 190:3783-3785; Frier et al., 1986 Proc. Nat. Acad. Sci. USA, 83 :9373-9377) “Perfectly complementary” means that all the contiguous residues of a nucleic acid molecule will hydrogen bond with the same number of contiguous residues in a second nucleic acid sequence.

In one embodiement, the siRNA molecules of the present invention are chemically modified to confer increase stability against nuclease degradation but retain the ability to bind to the target nucleic acid that is present in a cell. Modified siRNA of the present invention comprises modified ribonucleotide, and are resistant to enzymatic degradation such as RNAse degradation, yet they retain their ability to reduce virus infectivity by decreasing Staufen expression in a target cell. The siRNA may be modified at any position of the molecule so long as the modified siRNA is still capable of binding to the target sequence and is more resistant to enzymatic degradation. Modifications in the siRNA may be in the nucleotide base (i.e., purine or pyrimidine), the ribose or phosphate.

More specifically, the siRNA may be modified in at least one purine, in at least one pyrimidine or a combination thereof. Generally, all purines (adenosine or guanine) or all pyrimidine (cytosine or uracyl) or a combination of all purines and all pyrimidines of the siRNA are modified. Ribonucleotides on either one or both strands of the siRNA may be modified.

Non-limiting examples of chemical modification that can be included in an siRNA molecule include phosphorothioate internucleotide linkages (see US 2003/0175950), 2′-O-methyl ribonucleotides, 2′-O-methyl modified ribonucleotides, 2′-deoxy-2′-fluoro ribonucleotides, 2′-deoxy-2′-fluoro modified pyrimidines nucleotides, 5-C-methyl nucleotides and deoxyabasic residue incorporation. The ribonucleotides containing pyrimidine bases can be modified at the 2′ position of the ribose residue. A preferable modification is the addition of a molecule from the halide chemical group such as fluorine: Other chemical moieties such as methyl, methoxymethyl and propyl may also be added as modifications (see International PCT publication No. WO2004/011647). These chemical modifications, when used in various siRNA constructs, are shown to preserve RNAi activity in cells while at the same time, dramatically increasing there stability in cells or serum. Chemical modifications of the siRNA of the present invention can also be used to improve the stability of the interaction with the target RNA sequence.

siRNA of the present invention may also be modified by the attachment of at least one receptor binding ligand to the siRNA. Receptor binding ligand can be any ligand or molecule that directs the siRNA of the present invention to a specific target cell (e.g., T-lymphocytes and macrophages). Such ligands are useful to direct delivery of siRNA to a target cell in a body system, organ or tissue of a subject such has cells infected by a RNA virus. Receptor binding ligand may be attached to one or more siRNA ends, including any combination of 5′ or 3′ ends. The selection of an appropriate ligand for delivering siRNAs depends on the cells, tissues or organs that are targeted and is considered to be within the ordinary skill of the art. For example, to target an siRNA to hepatocytes, cholesterol may be attached at one or more ends, includind 3′ and 5′ ends. As another example, siRNA molecules can be targeted to T lymphocytes by attaching at the 3′ end or 5′ end of a siRNA molecules a HIV-1 surface antigen capable of binding to the CD4 surface protein located on T-cells (Kilby M. et al., 2003, New England J.of medicine, 348(22):2228-2238; Song, E. et al., 2003, J. of Virology, 77(13):7174-7181). Other conjugates such as other ligands for cellular receptors (e.g., peptides derived from naturally occurring protein ligands), protein localization sequences (e.g., ZIP code sequences), antibodies, nucleic acid aptamers, vitamins and other cofactors such as N-acetylgalactosamine and folate, polymers such as polyethyleneglycol (PEG), polyamines (e.g., spermine or spermidine) and phospholipids can be linked (directly or indirectly) to the siRNA molecule for improving its bioavailability.

siRNAs can be prepared in a number of ways well known in the art, such as by chemical synthesis, T7 polymerase transcription, or by treating long double stranded RNA (dsRNA) prepared by one of the two previous methods with Dicer enzyme. Dicer enzyme create mixed population of dsRNA from about 21 to 23 base pairs in length from double stranded RNA that is about 500 base paires to about 1000 base pairs in size. Dicer can effectively cleave modified strands of dsRNA, such as 2′-fluoromodified dsRNA (see WO2004/011647).

In one embodiment, vectors are employed for producing siRNAs by recombinant techniques. Thus, for example, a DNA segment encoding an siRNA derived from a Staufen sequence (e.g., SEQ ID 5, SEQ ID NO:1, SEQ ID NO:7, SEQ ID NO:3, SEQ ID NO:10, and SEQ ID NO:6) may be included in anyone of a variety of expression vectors for expressing any DNA sequence derived from a Staufen sequence. Such vectors include synthetic DNA sequences (e.g., derivatives of SV40, bacterial plasmids, baculovirus, yeast plamids, viral DNA such as vaccinia, fowl pox virus, adenovirus, lentivirus, retrovirus, adeno-associated virus, alphavirus etc), chromosomal, and non chromosomal vectors. Any vector may be used in accordance with the present invention as long as it is replicable and viable in the desired host. The DNA segment in the expression vector is operatebly linked to an appropriate expression control sequence(s) (e.g., promoter) to direct siRNA synthesis. Preferably, the promoters of the present invention are from the type III class of RNA polymerase III promoters (e.g., U6 and H1 promotors). The promoters of the present invention may also be inducible, in that the expression may be turned on or turned off (e.g., tetracycline-regulatable system employing the U6 promotor to control the production of siRNA targeted to Staufen).

In a particular embodiment, the present invention utilizes a vector wherein a DNA segment encoding the sense strand of the RNA polynucleotide is operatebly linked to a first promoter and the antisense strand of the RNA polynucleotide is operably linked to a second promoter (i.e., each strand of the RNA polynucleotide is independently expressed).

In another embodiment, The DNA segment encoding both strands of the RNA polynucleotide are under the control of a single promoter. In a particular embodiment, the DNA segment encoding each strand are arranged on the vector with a loop region connecting the two DNA segments (e.g., sense and antisense sequences), where the transcription of the DNA segments and loop region creates one RNA transcript. When transcribed, the siRNA folds back on itself to form a short hairpin capable of inducing RNAi (e.g., SEQ ID NO:30). The loop of the hairpin structure is preferably from about 4 to 6 nucleotides in length. The short hairpin is processed in cells by endoribonucleases wich removes the loop thus forming a siRNA molecule. In this particular embodiment, siRNAs of the present invention comprising a hairpin or circular structures are about 35 to about 65 nucleotides in length (e.g., 35, 36, 37, 38, 49, 40, 41, 42, 43, 44, 45, 46, 47, 48, 49, 50, 51, 52, 53, 54, 55, 56, 57, 58, 59, 60, 61, 63, 64, 65 nucleotides in length), preferably between 40 and 64 nucleotides in length comprising for example about 18, 19, 20, 21, 22, or 23, 24,25 base pairs.

In yet a further embodiment, the vector of the present invention comprises opposing promoters. For example, the vector may comprise two RNA polymerase III promoters on either side of the DNA segment (e.g., a specific Staufen DNA segment) encoding the sense strand of the RNA polynucleotide and placed in opposing orientations, with or without a transcription terminator placed between the two opposing promoters.

Non-limitimg examples of expression vectors used for siRNA expression are described in Lee et al., 2002, Nature Biotechnology, 19:505; Miyagishi and Taira, 2002, Nature biotechnology, 19:497; Pau et al., 2002, Nature Biotechnology, 19:500 and Novina et al., 2002, Naure Medecine, July 8(7):681-686).

The present invention also relates to antisense nucleic acid molecules which can be used for example to decrease or abrogate the expression of Staufen. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845, and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In one embodiment, antisense approach of the present invention involves the design of oligonucleotides (either DNA or RNA) that are complementary to Staufen mRNA. The antisense oligonucleotides bind to Staufen mRNA and prevent its translation. Absolute complementarity, althought preferred, is not absolutely a prerequisite. One skilled in the art can identify a certain'tolerable degree of mismatch by use of standard methods to determine the melting point of the hybridized antisense complex. In general, oligonucleotide that are complementary to the 5′untranslated region (up to the first AUG initiator codon) of Staufen mRNA should work more efficiently at inhibiting translation and production of Staufen protein. However, oligonucleotides that are targeted to a coding portion of the sequence may produce inactive truncated protein or diminish the efficiency of translation thereby lowering the overall expression of Staufen protein in a cell. Antisense oligonucleotides targeted to the 3′ untranslated region of messages have also proven to be efficient in inhibiting translation of targeted mRNAs (Wagner, R. (1994), Nature, 372:333-335). The Staufen antisense oligonucleotides of the present invention are less than 100 nucleotides in lenght, particularly, less than 50 nucleotides in length and more particularly less 30 nucleotides in length. Generally, effective antisense oligonucleotides are at least 15 or more oligonucleotides in length.

The antisense oligonucleotides of the present invention can be DNA, RNA, Chimeric DNA-RNA analogue, and derivatives thereof (see Inoue et al. (1987), Nucl. Acids. Res. 15: 6131-6148; Inoue et al. (1987), FEBS lett. 215: 327-330; Gauthier at al. (1987), Nucl. Acids, Res. 15: 6625-6641.). As mentioned above, antisense oligonucleotides of the present invention may include modified bases or sugar moiety. Examplary modified bases' include xanthine, hypoxanthine, 2 methyladenine, N6-isopentenyladenine, 2-methylguanine, 3-methylcytosine, 5-methylcytosine, N6-adenine, 7-methyguanine, 5-fluorouracil, 5-chlorouracil, 5-bromouracil, 5-iodouracyl, 5-carboxymethylaminomethyluracil, 5-methoxycarboxymethyluracil, queosine, 4-thiouracil and 2,6-diaminopurine. Examples of modified sugar moieties include hexose, xylulose, arabinose and 2-fluoroarabinose. The antisense oligonucleotide of the present invention may also include modified phosphate backbone such as methylphosphonate, phosphoramidate, phosphoramidothioates, phosphordiamidate and alkyl phosphotriesters. The synthesis of modified oligucleotides can be done according to methods well known in the art.

Once an antisense oligonucleotide or siRNA is designed, its effectiveness can be appreciated by conducting in vitro studies that assess the ability of the antisense to inhibit gene expression (e.g., Staufen protein expression). Such studies ultimately compare the level of Staufen RNA or protein with the level of a control experiment (e.g., an oligonucleotide wich is the same has that of antisense experiment but being a sense oligonucleotide or an oligonucleotide of the same size as the antisense oligonucleotide but that does not bind to a specific Staufen sequence).

The present invention generally relates to Staufen expression modulation and the use of Staufen expression modulation (i.e. Staufen overexpression, and Staufen expression inhibition) to treat or prevent of RNA viruses infections such as HIV infections. In one particular embodiment, the modulation of Staufen expression is carried out by using siRNAs or antisense sequences. In another particular embodiment, the invention relates to gene therapy to modulate Staufen gene expression.

As used herein, the term “gene therapy” relates to the introduction and expression in an animal (preferably a human) of an exogenous sequence (e.g.; a Staufen gene or cDNA sequence, a Staufen siRNA or antisense nucleic acid) to supplement, replace or inhibit a target gene (i.e., Staufen gene), or to enable target cells to produce a protein (e.g., a Staufen chimeric protein to target a specific molecule to HIV virion) having a prophylactic or therapeutic effect toward RNA virus infection such as HIV infection.

Non virus-based and virus-based vectors (e.g., adenovirus- and lentivirus-based vectors) for insertion of exogenous nucleic acid sequences into eukaryotic cells are well known in the art and may be used in accordance with the present invention. Virus-based vectors (and their different variations) for use in gene therapy have already been described in details (see). In virus-based vectors, parts of a viral gene are replaced by the desired exogenous sequence so that a viral vector is produced. Viral vectors are no longer able to replicate due to DNA manipulations.

In one specific embodiment, lentivirus derived vectors are used to target a Staufen sequence (e.g., siRNA, antisense, nucleic acid encoding a partial or complete Staufen protein) into specific target cells (e.g., T-lymphocytes or macrophages of a patient infected with HIV). These vectors have the advantage of infecting quiescent cells (for example see U.S. Pat. No. 6,656,706; Amado et al., 1999, Science 285: 674-676).

In addition to a Staufen nucleic acid sequence, siRNA or antisense, the vectors of the present invention may contain a gene that acts as a marker by encoding a detectable product.

Since overexpression of Staufen and inhibition of Staufen expression both cause a decrease in infectivity of RNA viruses and since it is incorporated in such viruses, the use of a vaccine against Staufen should be an alternative and/or complementary way to treat a RNA virus infection. In the specific case of a Staufen vaccine, the Staufen exogenous sequence may be linked to other molecules including diphtheria toxin, other immunogenic toxin peptides or helper antigen peptides in order to improve its efficiency in eliciting the desire immunological response in vivo. Humanized mouse monoclonal antibodies or DNA vaccines comprising a Staufen nucleic acid sequence or fragment thereof (for an example on DNA vaccines see U.S. Pat. No. 6,472,375) may be used in accordance with the present invention to treat RNA virus infections.

The vectors of the present invention may contain an IRES (internal ribosome entry site). An IRES will be used in circumstances that one wants to express two proteins from the same promoter. For example Staufen protein and a marker gene or a further therapeutic agent. When using an IRES the expression of the two proteins is coordinated. A further gene or genes may also be present under the control of a separate promotor. Other components may be included to serve certains functions, for example for directing the nucleic to a certain location in the cell.

One way of performing gene therapy is to extract cells from a patient, infect the extracted cells with a viral vector and reintroduce the cells back into the patient. A selectable marker may or may not be included to provide a means for enriching for infected or transduced cells. Alternatively, vectors for gene therapy that are specially formulated to reach and enter target cells may be directly administered to a patient (e.g., intravenously, orally etc.).

The exogenous sequences (e.g., antisense RNA, si RNA, a nucleic acid encoding Staufen of parts thereof) may be delivered into cells that express Staufen according to well known methods. Apart from infection with virus-based vectors, examples of methods to deliver nucleic acid into cells include DEAE dextran lipid formulations, liposome-mediated transfection, CaCl₂-mediated transfection, electroporation or using a gene gun. Synthetic cationic amphiphilic substances, such as dioleoyloxypropylmethylammonium bromide (DOTMA) in a mixture with dioleoylphosphatidylethanolamine (DOPE), or lipopolyamine (Behr, Bioconjugate Chem., 1994 5:382), have gained considerable importance in charged gene transfer. Due to an excess of cationic charge, the substance mixture complexes with negatively charged genes and binds to the anionic cell surface. Other methods include linking the exogenous oligonucleotide sequence (e.g., siRNA, antisense, Staufen nucleic acid or fragment thereof) to peptides or antibodies that especially binds to receptors or antigens at the surface of a target cell. U.S. Pat. No. 6,358,524 describe target cell-specific non-viral vectors for inserting at least one gene into cells of an organism. The method described the uses of non-viral carriers that are cationized to enable them to complex with the negatively charged DNA. Moreover, the method also includes the use of a ligand (e.g., a monoclonal antibody or fragment thereof that is specific for membrane antigen on macrophages or lymphocytes, glycoproteins of the coats of viruses etc.) which can specifically bind to the desired target cell in order to enter it.

To achieve high cellular concentration of the Staufen nucleic acid sequence, the antisense nucleic acid or small inhibitor RNAs of the present invention an effective method utilizes a recombinant DNA construct in which the nucleic acid sequence is placed under a strong promoter and the entire construct is targeted into the cell. Such promoters may constitutively or inducibly produce Staufen or part thereof, the antisense RNA or siRNA of the present invention.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a encapsulation in liposome or nanoerythrosome or by incorporation in other vehicles, such as hydrogels, cyciodextrins, biodegradable nanocapsules and bioadhesive microsphere or by proteinacious vectors (PCT No. WO 00/53772) which can be designed to be targeted to a specific cell type, and engineered to be administered through different routes. Methods for the delivery of nucleic acid molecules are described in Akthar et al., 1992, trends Cell Bio., 2:139; Delivery strategies for antisense oligonucleotide therapeutics, ed. Akhtar, 1995; Maurer et al., 1999, Mol. Membr. Biol., 16:129-140; Hoflamd et Huang, 1999, Handb. Exp. Pharmacol., 137:165-192; and Lee et al., 2000, ACS symp. Ser., 752:184-192. Delivery of nucleic acid molecules of the present invention (e.g., Staufen nucleic acid sequence, siRNAs and antisense RNAs) to target cells such as T-cells can be accomplished by methods well as known in the art (see e.g., Rudoll et al., 1996, Gene Therapy, 3(8):695-705; Smith at al., 1996, Antiviral Research, 32(2):99-115; Phillips et al., 1996, Nature Medecine 2(10):1154-1156 and U.S. Pat. No. 6,622,854). Beigelman et al., U.S. Pat. No. 6,395,713 and Sullivan et al., PCT WO 94/02595, further describes the general methods for delivery of nucleic acid molecules.

The amount of the therapeutic or pharmaceutical composition (e.g., antiviral agent such as Staufen antisenses, Staufen protein, Staufen fusion/chimeric protein, siRNAs and the like) which is effective in the treatment of a particular disease, disorder or condition (e.g., RNA virus infection, HIV infection) will depend on the nature and severity of the disease, the chosen therapeutic regimen (i.e DNA construct, protein, cells), the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.01 to 100 mg/kg of body weight/day will be administered to the subject depending on the potency of the negatively charged polymer. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In vivo administration of nucleic acid constructs, virus stocks or cells which have been transduced with the constructs of the present invention is to be determined by dose escalation, with the upper dose being limited by the onset of unacceptable adverse effects. Preliminary starting doses may be extrapolated from experiments using the same type of vectors (e.g., lentivirus based vectors or non-virus based vectors) in animal models. Generally, small dosages will be used initially and if necessary, will be increased by small increments until the optimum effect under the circumstances is reached. Examplary dosages are within the range of 10⁸ to 5×10¹⁵ particles.

In the case of vaccines, the chosen route of administration will depend on the vaccine composition and the disease status of the patient. Relevant considerations include the types of immune cells to be activated, the time during which the antigen is exposed to the immune system and the immunization schedule. Althought many vaccines are administered consecutively within a short period of time, spreading the immunization over a longer period may maintain effective clinical and immunological responses.

A composition within the scope of the present invention should contain the active agent (i.e.Staufen recombinant protein or fragments thereof, Staufen fusion/chimeric protein, nucleic acids such as siRNAs, antisenses and the like), in an amount effective to achieve an inhibitory effect on RNA viruses including HIV and related viruses while avoiding adverse side effects. Pharmaceutically acceptable preparations and salts of the active agent are within the scope of the present invention and are well known in the art (Remington: The Science and Practice of Pharmacy by Alfonso R. Gennaro, 2003, 21^{th} edition, Mack Publishing Company.).

For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 100 mg/kg/day will be administered to the mammal.

The pharmaceutical composition of the present invention can be administered by any suitable route including, intrevanous or intramuscular injection, intraventricular or intrathecal injection (for central nervous system administration), orally, topically, subcutaneously, subconjunctivally, or via intranasal, intradermal, sublingual, vaginal, rectal or epidural routes.

Other delivery system well known in the art can be used for delivery (and/or targeting) of the pharmaceutical compositions of the present invention, for example via aqueous solutions, encapsulation in microparticles, liposomes, or microcapsules.

In yet another embodiment, the pharmaceutical compositions of the present invention can be delivered in a controlled release system. In one embodiment polymeric materials can be used (see Smolen and Ball, Controlled Drug Bioavailability, Drug product design and performance, 1984, John Wiley & Sons; Ranade and Hollinger, Drug Delivery Systems, pharmacology and toxicology series, 2003, 2nd edition, CRRC Press), in another embodiment, a pump may be used (Saudek et al. (1989), N. Engl. J. Med. 321: 574).

Compounds (e.g., antiviral agent for infection with RNA viruses such Xas HIV) of the present invention may also be delivered by the use of monoclonal antibodies as individual carriers to which the compound molecules are coupled. The compounds of the present invention may also be coupled to a class of biodegradable polymers useful in achieving controlled release of the drug, for example, polylactic acid, polyorthoesters, cross-linked amphipathic block copolymers and hydrogels, polyhydroxy butyric acid and polydihydropyrans.

Pharmaceutical compositions of the present invention comprise at least one antiviral agent (e.g., Staufen antisense RNA, siRNA, Staufen protein, Staufen fusion/chimeric protein and the like) combined with a pharmaceutically acceptable carrier. The term carrier refers to diluents adjuvants, excipients or vehicles with which the antiviral agent is administered. Such pharmaceutical carriers include sterile liquids such as water and oils including mineral oil, vegetable oil (e.g., peanut oil, soybean oil, sesame oil), animal oil or oil of synthetic origin. Aqueous glycerol and dextrose solutions as well as saline solutions may also be employed as liquid carriers of the pharmaceutical compositions of the present invention. Of course, the choice of the carrier depends on the nature of antiviral compound, its solubility and other physiological properties as well as the target site of delivery and application. For example, the Staufen antisense nucleic acid or siRNA can be combined with a lipophilic carrier such as any one of a number of sterols including cholesterol, cholate and deoxycholic acid. In one embodiment, the sterol is cholesterol. Examples of suitable pharmaceutical carriers are described in Remington: The Science and Practice of Pharmacy by Alfonso R. Gennaro, 2003, 21^{th} edition, Mack Publishing Company.

Further pharmaceutically suitable materials that may be incorporated in pharmaceutical preparations of the present invention include absorption enhancers, pH regulators and buffers, osmolarity adjusters, preservatives, stabilizers, antioxidants, surfactants, thickeners, emollient, dispersing agents, flavoring agents, coloring agents and wetting agents all of which being well known in the art.

Examples of suitable pharmaceutical excipients include, water glucose, sucrose, lactose, glycol, ethanol, glycerol monostearate, gelatin, rice, starch flour, chalk, sodium stearate, malt, sodium chloride and the like. The pharmaceutical compositions of the present invention can take the form of solutions, capsules, tablets, creams, gels, powders sustained release formulations and the like. The composition can be formulated as a suppository, with traditional binders and carriers such as triglycerides (see Remington: The Science and Practice of Pharmacy by Alfonso R. Gennaro, 2003, 21^{th} edition, Mack Publishing Company). Such compositions contain a therapeutically effective amount of the therapeutic composition, together with a suitable amount of carrier so as to provide the form for proper administration to the subject. The formulations are designed so as to suit the mode of administration and the target site of action (e.g., a particular organ or cell type).

The pharmaceutical compositions of the present invention can be formulated as neutral or salt forms. Pharmaceutically acceptable salts of proteins or peptides (e.g., Staufen protein, Staufen fusion/chimeric protein) include those that form with free amino groups and those that react with free carboxyl groups. Non-toxic alkali metal, alkaline earth metal and ammonium salts commonly used in the pharmaceutical industry include sodium, potassium, lithium, calcium, magnesium, barium, ammonium, and protamine zinc salts, which are prepared by methods well known in the art. The term also includes non-toxic acid addition salts, which are generally prepared by reacting the compounds of the present invention with suitable organic or inorganic acid. Representative salts include the hydrobromide, hydrochloride, valerate, oxalate, oleate, laureate, borate, benzoate, sulfate, bisulfate, acetate, phosphate, tysolate, citrate, maleate, fumarate, tartrate, succinate, napsylate salts and the like.

The pharmaceutical compounds of the present invention may be administered alone or in combination with other active agents useful for the treatment, prophylaxis or amelioration of symptoms of RNA viruses associated disease or condition. Thus, the compositions and methods of the present invention can be used in combination with other agents exhibiting the ability to inhibit or reduce RNA viruses activity (e.g., infectivity, replication, assembly, morphogenesis and the like). Example of such agents include but are not limited to antiretroviral agents such as zidovudine, didanosine, tenofovir, nevirapine, TMC-125, nelfinavir, tripanavir, atazanatir, stavudine, zalcitabine, abacavir and efavirenz.

The present invention is illustrated in further details by the following non-limiting example. The examples are provided for illustration only and should not be construed as limiting the scope of the invention.

### EXAMPLE 1

### Molecular Cloning and Sequencing of the cDNAs

In order to clone a human Staufen homologue, the GenBank database was searched with Drosophila dsRNA-binding domain sequences to find consensus sequences and eventually design degenerate oligonucleotide primers for RT-PCR. However, searching in the expressed sequence tags (EST) database identified a partial sequence, clone HFBDQ83 (GenBank accession number T06248), with high homology to the Drosophila sequence. This clone was purchased from the American Type Culture Collection and used as a probe to screen both human brain (Clontech) and foetal total mouse (a generous gift from A. Royal) cDNA libraries as described previously (Wickham et al. (1991), DNA and Cell biology 10: 249-258). DNA from the isolated λGT10 clones was subcloned into a Bluescript™ vector (Stratagene). Double-stranded DNA was sequenced by the dideoxynucleotide method, according to Sequenase TMprotocols (United States Biochemical Corp.).

### EXAMPLE 2

### Construction of Fusion Proteins

The 1.2 kbp BamHI fragment of the human HFBDQ83 cDNA was subcloned in frame in either pQE32 (Qiagen) or pMAL-c (New England Biolabs) thus generating the protein fused to a hexahistidine tag or to the maltose-binding protein (MBP), respectively. The protein was expressed in bacteria by inducing with IPTG, as recommended by the manufacturer. Full-length and internal fragments of the mStau protein were PCR-amplified and cloned into pMal-c to produce fusion proteins with the maltose-binding protein. For the expression of the internal domains, which do not contain an endogenous stop codon, the PCR fragments were cloned in a modified pMal-c vector (pMal-stop) in which stop codons were introduced at the HindIII site, by the ligation of the annealed complementary oligonucleotides 5′-AGCTTAATTAGCTGAC-3′ (SEQ ID NO:13) and 5′-AGCTGTCAGCTMTTA-3′ (SEQ ID NO:14). The MBP/mSTAU fusion protein, containing the full-length mStau sequence, was generated by PCR amplification with Vent DNA polymerase (New England BioLabs), using the primer pair 5′-CCTGGATCCGMAGTATAGCTTCTACCATTG-3′ (SEQ ID Nb:15) and 5′-TACATMGCTTCTAGATGGCCAGAAAAGGTTCAGCA-3′ (SEQ ID NO:16). The resulting 1562 bp fragment was digested with HindIII and BamHI, and ligated in the pMal-c vector. The C-terminal fragment (mSTAU-C) was amplified with the primer pair 5′-GGATGAATCCTATTAGTAGACTTGCAC-3′ (SEQ ID NO:17) and 5′-TACATAAGCTTCTAGATGGCCAGAAAAGGTTCAG-CA-3′ (SEQ ID NO:23), digested with HindIII and cloned in the EagI* and HndIII sites of pMal-c. Eagl* was created by filling in the cohesive ends of Eagl-digested pMal-c vector using the Klenow fragment of DNA polymerase 1. This fusion vector was then digested with SacI and EcORI and the resulting fragment was subcloned in the pMal-stop vector to generate the mSTAU-RBD3 construct. The mSTAU-TBD construct was prepared by PCR using the primer pair 5′-GCTCTAGATTCAAAG-TTCCCCAGGC-GCAG-3′ (SEQ ID NO:18) and 5′-TTTAAGCTTCTCAGA-GGGTCTAGT-GCGAG-3′ (SEQ ID NO:19); the product was digested with XbaI and HindIII and cloned in the pMal-stop vector. mSTAU-RBD2 and mSTAU-RBD1 were constructed by first amplifying a fragment using the primer pair 5′-CAATGTATMGCCCGTGGACCC-3′ (SEQ ID NO:20) and 5′-AAAAAGCTTGTGCMGTCTACTMTAGGATTCACC-3′ (SEQ ID NO:21). The resulting product was digested with HindIII and cloned in the EagI* and HindIII sites of the pMal-stop vector. This vector was then used to purify the 398 bp PstI and HindIII fragment, which was subcloned in the pMAL-stop vector to generate the mSTAU-RBD2 construct. In the same way, the mSTAU-RBD1 vector was obtained by digestion with SmaI and StuI, followed by recircularization of the digestion product using T4 DNA ligase. The mSTAU-RBD4 was PCR amplified using the primer pair 5′-ATAGCCCGAGAGTTGTTG-3′ (SEQ ID NO:22) and 5′-TACATMGCTTCTAGATGGC-CAGAAAAGGTTCAGCA-3′ (SEQ ID NO:23). The resulting fragment was digested with HindIII and ligated in the pMal-stop vector at the StuI and HindIII sites. All the MBP/Staufen fusion plasmids were transformed in the BL-21 E. coli strain. The fusion proteins were obtained after induction with 1 mM IPTG for 2-3 hours. Cells were lysed in SDS-PAGE loading buffer for immediate use, or frozen at −80οC for storage.

### EXAMPLE 3

### Antibody Production and Western Blotting

For the production of antibodies, a large amount of the his/hStau fusion protein was purified on Ni-NTA resin (Qiagen), as recommended by the manufacturers, and injected into rabbits, as done previously (Aloyz et al. (1995), Peptides 16, 331-338). For western blotting, cells were lysed in 1% n-octylglucosid, 1 mM PMSF, 1 mg/ml aprotinin and 1 mg/ml pepstatin A in PBS. Protein extracts were quantified by the Bradford method (Bio-Rad), and similar amounts of proteins were separated on 10% SDS-polyacrylamide gels and transferred onto nitrocellulose membranes. Membranes were blocked for 30 min in TBS (Tris-buffered saline) plus 5% dry milk and incubated with primary antibodies in TBS plus 0.05% Tween for 1 hr at room temperature. Detection was accomplished by incubating the blots with peroxydase-conjugated anti-rabbit immunoglobulin antibodies (Dimension Labs) followed by Supersignal™ Substrate (Pierce), as recommended by the manufacturer.

### EXAMPLE 4

### RNA-Binding Assay

Bacterial extracts from IPTG-induced cultures were separated on 10% SDS-polyacrylamide gels and the proteins transferred onto nitrocellulose membranes. Membranes were incubated in the presence of [³²P]-labeled RNA probes in 50 mM NaCl, 10 mM MgCl₂, 10 mM Hepes, pH 8.0, 0.1 mM EDTA, 1 mM DTT, 0.25% milk, for 2 hr at room temperature, washed in the same buffer for 30 min, and exposed for autoradiography. For competition assays, an excess of cold homopolymers (Pharmacia) was added to the hybridization mixture along with the labeled probe. The 3′-UTR of bicoid cDNA (position 4016 to 4972) which was PCR-amplified from Drosophila genomic DNA, and subcloned in the bluescript™ vector, was transcribed using T7 RNA polymerase in the presence of [α-³²P]CTP. Synthetic RNAs (Pharmacia) were labeled with T4 polynucleotide kinase in the presence of [δ-³²P]ATP.

### EXAMPLE 5

### Tubulin-Binding Assay

Bacterial extracts from IPTG-induced cultures were separated on 10% SDS-polyacrylamide gels and the MBP-tagged proteins were transferred onto nitrocellulose membranes. Membranes were incubated in 10 mM Tris, pH 8.0,150 mM NaCl (TBS) and 1% Tween 20 for 45 min prior to an overnight overlay with 7 mg/ml tubulin (Sigma) in TBS plus 0.2% Tween 20. Blots were washed several times in TBS plus 0.2% Tween 20, and then incubated with a mixture of mouse monoclonal anti-α- and anti-β-tubulin antibodies (ICN). Bound antibodies were detected with secondary peroxydase-conjugated anti-mouse immunoglobulin antibodies (Dimension Labs) and Supersignal Substrate (Pierce), as stated previously. Separate assays were performed with actin and anti-actin antibodies (both from Sigma).

### EXAMPLE 6

### Cell Culture and Transfection

293T cells were grown at 37° C. in Dulbecco's modified Eagles Medium containing 8% fetal bovine serum and antibiotics (Gibco BRL, Invitrogen). All transfections were performed using the calcium phosphate-mediated co-precipitation method as reported (Mouland et al. (2000), J. Virol. 74:5441-5451) except that a lipofection technique was used for the siRNA experiments described below.

### EXAMPLE 7

### Immunofluorescence

Hstau/HA and hStau/GFP were constructed by PCR-amplification of the full-length cDNA using the primer pair 5′-TACATGTCGACTTCCTGCCA/GGGCTGCGGG-3′ (SEQ ID NO:24) and 5′-TACMTCTAGATTATCAGCGGCCGCACCTCCCACACACAGAC-AT-3′ (SEQ ID NO:25). The 3′-primer was synthesized with a NotI site just upstream from the stop codon allowing ligation of a NotI cassette containing either three copies of the HA-tag or the GFP sequence. The resulting fragment was cloned in Bluescript™ following digestion with SalI and XbaI. The KpnI/XbaI fragment was then subcloned in the pcDNA3/RSV vector (Jockers et al. (1996), J. Biol. Chem. 271, 9355-9362) and a NotI-cassette was introduced at the NotI site. For the TBD/GFP fusion protein, the TBD was PCR-amplified with oligonucleotides on each side of this region (SEQ ID NO: 26 5′-TACATMGCTTMGCCACCATGGTCAAAGTTCC—CCAGGCGC-3′) and (SEQ ID NO:27 5′-TACMTC-TAGAGCGGCCGCGCT-CAGAGGGTCTAGTGCGAG-3′). The sense primer contained an ATG initiation codon and the so called Kozak consensus sequence, upstream from the TBD sequence. The anti-sense primer contained a NotI site, just upstream from a stop codon. The resulting fragment was digested with HindIII and XbaI and cloned into the pcDNA3/RSV vector. The GFP NotI-cassette was then introduced at the NotI site.

Mammalian cells were transiently transfected with the cDNAs by the calcium/phosphate precipitation technique, fixed in 4% paraformaldehyde in phosphate buffered-saline (PBS) for 25 min at room temperature and permeabilized with 0.3% Triton X-100 in PBS containing 0.1% BSA. The cells were then blocked with 1% BSA in PBS, 0.3% Triton X-100 and incubated with mouse anti-HA, rabbit anti-calreticulin or rabbit anti-calnexin antibodies for 1 hr at room temperature, as indicated. Cells were washed in permeabilization buffer and incubated with fluorescein-conjugated or Texas-Red-conjugated species-specific secondary antibodies (Jackson Immunoresearch Laboratories, West Grove, Pa.) in blocking buffer for 1 hr. GFP and GFP fusion proteins were detected by autofluorescence. Mounting was done in ImmunoFluor Mounting Medium (ICN). For the analysis of cytoskeleton-associated proteins, transfected cells were first extracted in 0.3% Triton X-100, 130 mM HEPES (pH 6.8), 10 mM EGTA, 20 mM MgSO₄ for 5 min at 4° C., as previously described (Davis et al. (1987), Nature 330, 477-479). They were then fixed in 4% paraformaldehyde in PBS and processed for immunofluorescence as described above. Cells were visualized by immunofluorescence using the 63× planApochromat objective of a Zeiss Axioskop fluorescence microscope.

Confocal microscopy was performed with the 60× Nikon Plan Apochromat objective of a dual channel BioRad 600 laser scanning confocal microscope equipped with a krypton/argon laser and the corresponding dichroid reflectors to distinguish fluorescein and Texas Red labeling. No overlap was observed between the fluorescein and Texas Red channels. Confocal images were printed using a Polaroid TX1500 video printer.

### EXAMPLE 8

### Immunoprecipitation Experiments

In immunoprecipitation experiments, 293T cells were either transfected with Staufen-expressing chimeric proviruses or co-transfected with cDNAs coding for Gag/Rluc and Staufen-HA fusion proteins depending on the type of assay. Forty hours post-transfection, cells were harvested, washed three times in PBS and lysed [100 mM NaCl, 10 mM Tris, 1 mM EDTA, 0.5% NP-40 and protease inhibitor cocktail (Roche)]. Staufen and Gag expression was monitored by Western blot analysis using mouse monoclonal anti-HA and anti-CA antibodies, respectively. For immunoprecipitation analysis, 3-5 mg of cell lysates was precleared with normal rabbit serum and 25 μL of a 50:50 slurry of protein A-Sepharose (Amersham Biosciences), incubated with rabbit polyclonal anti-CA antiserum (American BioTechnologies; dilution 1/200) for 90 minutes at 4° C. and with 25 μL of a 50:50 slurry of protein A-Sepharose for 1 h at 4° C. To immunoprecipitate HA-tagged proteins, lysates were incubated with rat monoclonal anti-HA antibody covalently coupled with agarose beads (Roche Applied Bioscience) for 4 h at 4° C.: In some cases, stringency of the immunoprecipitation was increased by adjusting the concentrations of NP-40 and SDS to 1% and 0.1%, respectively. Resulting IP-pellets were washed 4 times with lysis buffer and once with PBS. Immunoprecipitates were analysed by SDS-PAGE using mouse monoclonal anti-HA and anti-CA antibodies to detect Staufen and Gag in Western blot analyses. The immunoprecipitation of endogenous Staufen was performed essentially as described above for anti-CA except that a rabbit polyclonal antiserum was used (Mouland et al., 2000, supra). The same rabbit antiserum was used to identify Staufen in Western analyses.

### EXAMPLE 9

### Molecular Cloning of Mammalian Staufen cDNAs

In order to understand the mechanism of mRNA transport in mammalian cells, the human and mouse Staufen homologues was cloned. Thirteen overlapping human cDNAs, ranging in size between 0.8 and 2.5 kb, were isolated from a human central nervous system cDNA library, using the expressed sequence tag (EST) HFBDQ83 cDNA as a probe (FIG. 1A). Purified human HeLa cell poly(A)+ RNAs were also reverse transcribed and PCR-amplified using different 5′-RACE protocols, allowing the cloning of the 5′-end of the transcript. Two different cDNAs of 3217 and 3506 nucleotides were identified from overlapping clones (see below). The presence of multiple transcripts in human cells was confirmed by RT-PCR experiments (not shown). One of the human cDNAs was then used to screen a foetal total mouse cDNA library under low stringency conditions, which led to the isolation of a full-length cDNA (mStau)(GB accession number: AF061942). The nucleic and amino acid sequences of mStau Is shown in FIG. 1C. The human and mouse proteins are 90% identical (98% similarity), as shown in the alignment of the sequences thereof (FIG. 1D).

Hybridization of a Human Multiple Tissues Northern Blot with a human cDNA reveals that hStau mRNA is found in every tested tissue (FIG. 2A), unlike the Drosophila Staufen gene which is exclusively expressed in oocytes and in the CNS at the larval stage (St Johnston et al., 1991, supra). The size of the cDNAs is close to that of the transcripts, which migrate on a Northern blot as an unresolved large band of around 3.6 kb.

### EXAMPLE 10

### A Differential Splicing Event Generates Two Human Staufen Proteins

Characterization of the human cDNAs revealed the presence of four types of transcripts which only differ by an insertion of 289 bp at position 324 (FIGS. 1A and 1B). Interestingly, this sequence introduces an ATG initiation codon upstream from the first one found in the short transcript (FIG. 1A). This suggests that two putative proteins of 63 and 55 kDa may be translated, with one protein exhibiting an 81 amino acid extension at its N-terminal extremity, as compared to the other protein. Using anti-hStau antibodies in western blot experiments, two protein bands of around 63 and 55 kDa in human cell extracts were observed (FIG. 2B). To determine whether the cDNAs could account for the presence of the two proteins, each of them was subcloned in an expression vector and expressed in mammalian cells. As seen in FIG. 2C, each cDNA gives rise to a single overexpressed protein which perfectly comigrates with the endogenous proteins.

To determine whether these transcripts are the products of differential splicing, genomic DNA was PCR-amplified with primers located on each side of the insert. The resulting fragments were cloned, and their extremities sequenced. Comparison of the genomic and cDNA sequences demonstrated that the DNA insert is carried on a single exon, and that typical splicing consensus sequences are present at each intron/exon junction (not shown).

Taken together, these results demonstrate that the human Staufen gene produces two different transcripts by alternative splicing and exon skipping, and that the transcripts code for two highly homologous proteins which differ in their N-terminal extremities.

### EXAMPLE 11

### Comparison of the Mammalian and Drosophila Staufen Proteins

The amino acid sequences of the mammalian proteins are similar to that of the Drosophila Staufen protein and of the product of an uncharacterized ORF on the X chromosome of Caenorhabditis elegans (FIG. 2D and FIG. 1′). The overall structure and relative position of the full-length and short-RBDs are well conserved and high sequence identity is found between corresponding dsRBDs. This is highly significant since an alignment of the domains found in the members of the dsRNA-binding protein family shows an average of only 29% amino acid identity to one another (St Johnston et al., 1992, supra). In addition, domains 1 and 4 in the human sequence, which are short domains when compared to the consensus, are nevertheless highly similar to the corresponding fly sequences, even in the region that extends far beyond the N-terminal side of the consensus sequence, suggesting that they must play an essential role in Staufen function.

Mammalian Stau does not contain the first dsRNA-binding domain nor the long N-terminal sequence of the Drosophila protein which was shown to bind to oskarprotein (Breitwieser et al. (1996), Genes & Dev.10, 2179-2188). In addition, a putative tubulin-binding domain located between the third and fourth dsRNA-binding domains of mammalian Stau is not found in the Drosophila protein, at least at the amino acid level. This region contains a stretch of 91 amino acids which show 25% amino acid identity (66% similarity) to a microtubule-binding domain of MAP1B (Zauner et al. (1992), Eur. J. Cell Biol. 57, 66-74). It is meaningful that the sequence similarity covers the full microtubule-binding domain of MAP1B and that it is restricted to this domain.

### EXAMPLE 12

### The Human and Mouse Staufen Proteins Bind Double-Stranded RNAs

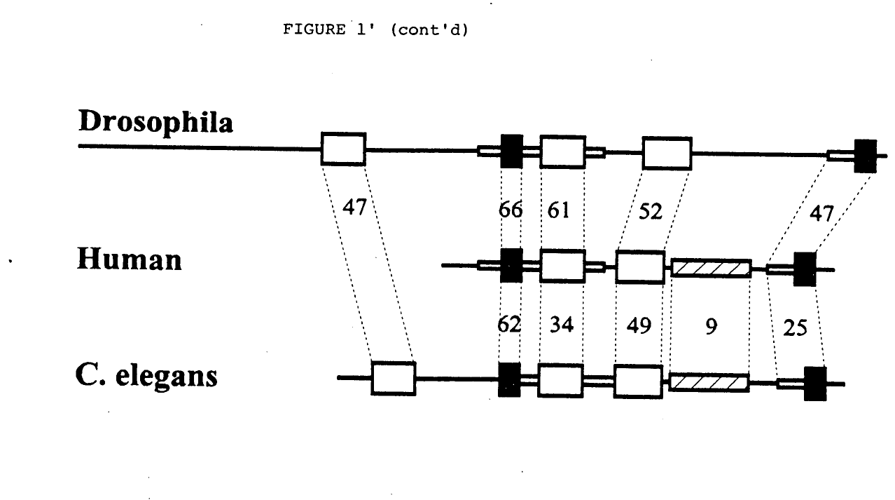

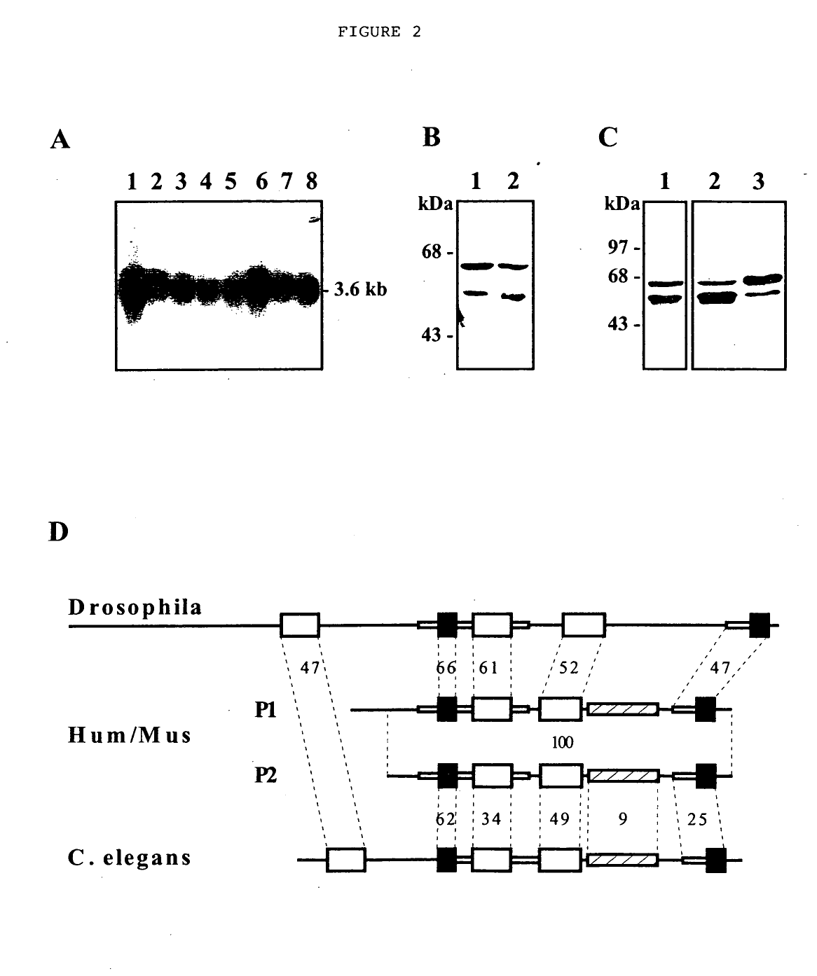

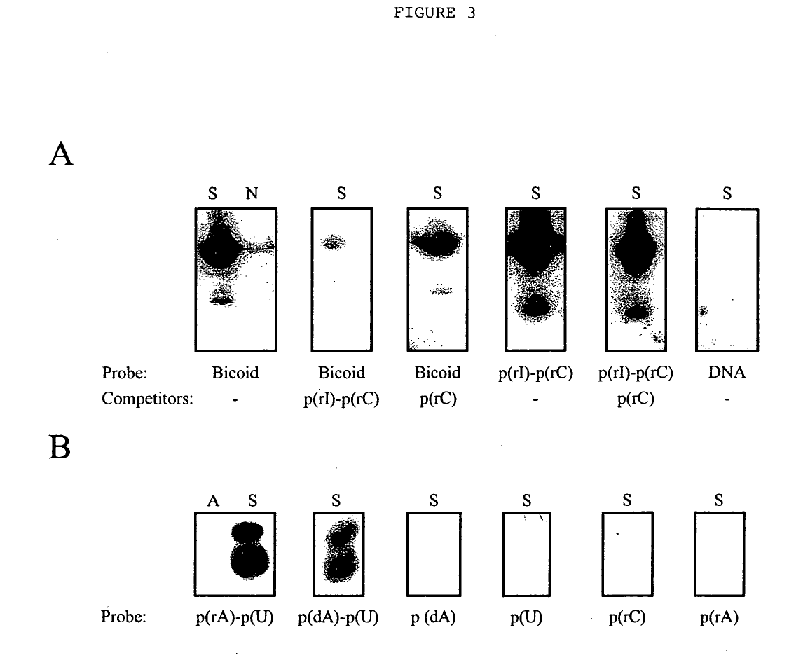

As seen in FIGS. 2D and 1′, mammalian Stau proteins contain multiple dsRNA-binding domains. In order to determine whether Stau binds RNAs, two bacterially-expressed fusion proteins were used in an RNA-binding assay, his/hStau and MBP/mStau. The fusion proteins were probed with in vitro-labeled bicoid mRNA, which is known to adopt an extensive secondary structure and to strongly bind to the Drosophila Staufen protein, both in vivo and in vitro (St Johnston et al., 1992, supra; Ferrandon et al., 1994, supra). Both fusion proteins strongly bind this RNA. The binding is competed by an excess of cold poly(rI)-poly(rC), but not by poly(rI), poly(rC), poly(rA) or poly(U), nor by tRNA or dsDNA (for example, see FIG. 3A), suggesting that mammalian Stau recognizes double-stranded structures in the RNA rather than a sequence-specific region. Both fusion proteins also directly bind labeled double-stranded RNAs and RNA/DNA hybrids, but not single-stranded RNA or DNA homopolymers (for example, see FIG. 3). As controls, a his/NEP (neutral endopeptidase) or MBP/aminopeptidase fusion proteins were also included on the blot; they did not bind any of these nucleic acids.

This demonstrates that both the human and mouse Staufen proteins, regardless of the protein to which they are fused, are able to bind dsRNAs and RNA with extensive secondary structure, as reported for the Drosophila protein (St Johnston et al., 1992, supra).

### EXAMPLE 13

### The Human and Mouse Staufen Proteins Bind Tubulin In Vitro

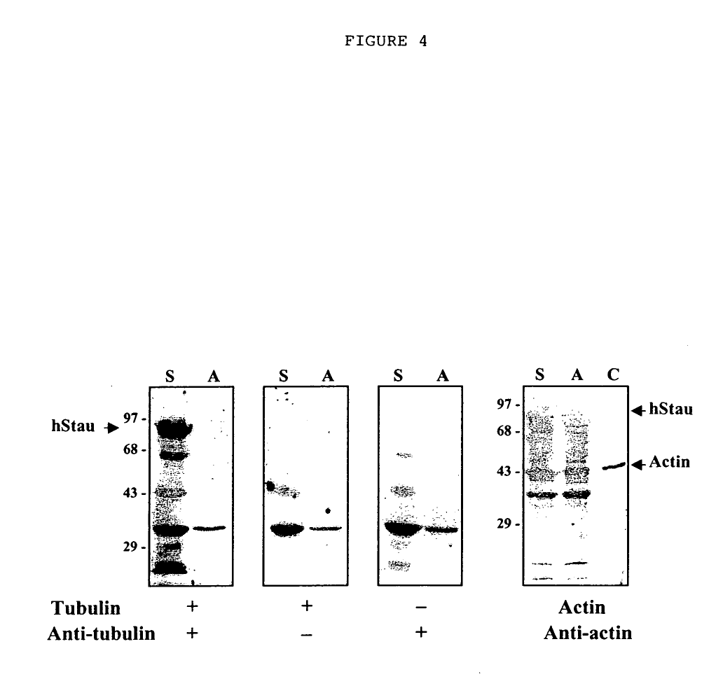

As described above, Stau contains a region which is similar to the microtubule-binding domain of MAP-1B. To determine whether mammalian Stau can bind tubulin, bacterially-expressed MBP/Stau fusion proteins were used in a tubulin-binding assay. As shown in FIG. 4, hStau binds tubulin in vitro. As a control, the MBP/aminopeptidase fusion protein was also included on the blot; it did not show any tubulin-binding capability. Under the same conditions, hStau cannot bind actin (FIG. 4), which suggests that the binding of tubulin to Staufen is specific. The same results were obtained with the MBP/mStau fusion protein (see FIG. 5B, lane 2). Binding to mRNAs and microtubules are two of the characteristics expected of localizing proteins, making hStau and mStau very good candidates for mRNA transport and localization in mammals.

### EXAMPLE14

### Molecular Mapping of the RNA- and Tubulin-Binding Domains

To determine which Staufen domain(s) is involved in RNA and/or tubulin binding, the MBP/mStau fusion protein was used to construct a series of deletion mutants (FIG. 5). The production and relative abundance of each fusion protein was first verified by Western blotting (not shown). Using the RNA-binding assay, it was demonstrated that both of the full-size dsRNA-binding domains (dsRBD2 and dsRBD3) are independently sufficient to bind bicoid RNA (FIG. 5A). In contrast, the two short-domains (dsRBD1 and dsRBD4) were unable to bind dsRNA in this assay. It was also demonstrated that the C-terminal half of mStau is able to bind tubulin (FIG. 5B, lane 4). More specifically, the region which is similar to the MAP1B-microtubule-binding domain is sufficient to bind tubulin (FIG. 5B, lane 6). The faint bands (FIG. 5B, lanes 3 and 5) were not reproducible.

These experiments confirm that the regions identified by sequence comparison as putative dsRNA- and tubulin-binding domains are biochemically functional.

### EXAMPLE 15

### Staufen is Associated with the Detergent-Insoluble Fraction In Vivo

The cellular distribution and cytoskeletal association of the two human Stau proteins in vivo was then addressed. To do so, the Green Fluorescent Protein (GFP) or an HA-tag were fused to the 63 and 55 kDa hStau isoforms, respectively. Using confocal microscopy, it was first shown that the two fusion proteins co-localize when co-expressed in mammalian cells (not shown). Then, it was shown that they are non-homogeneously distributed throughout the cytoplasm and label numerous vesicular and tubular structures which concentrate in the perinuclear region (FIG. 6A). Minimal staining was found in the nucleus. When the cells were treated with Triton X-100 prior to fixing, allowing soluble proteins to be separated from the cytoskeleton and cytoskeleton-associated proteins (Pachter, 1992, supra), the tubulovesicular labeling was still present, demonstrating that hStau is associated with the detergent-insoluble material in vivo (FIG. 6B). Labeled structures were also present in cell processes, suggesting that Stau may target mRNAs to peripheral ER elements. The same results were obtained following expression of the GFP/mStau protein (not shown). The association between hStau and the cytoskeletal-associated material was confirmed by in vitro cell fractionation in the presence of Triton X-100. In this assay, hStau partitioned mainly in the cytoskeleton-associated fractions, although a significant fraction was found in a soluble form, as judged by Western blotting (not shown).

To determine whether the tubulin-binding domain identified in vitro is truly involved in this function in vivo, mammalian cells were transfected with a cDNA coding for a fusion protein in which the minimal tubulin-binding domain was fused to GFP. In contrast to the full-length protein, the TBD/GFP fusion protein is randomly distributed in the cytoplasmic and nuclear domains of the cells (FIG. 6C), as is the GFP protein used as a control (FIG. 6D). This staining was completely extracted by the Triton X-100 treatment (not shown), suggesting that the minimal tubulin-binding domain found in vitro is not sufficient to render the protein insoluble and form a stable association with the microtubule network and/or the cytoskeleton-associated material.

### EXAMPLE 16

### Staufen Localizes to the Rough Endoplasmic Reticulum In Vivo

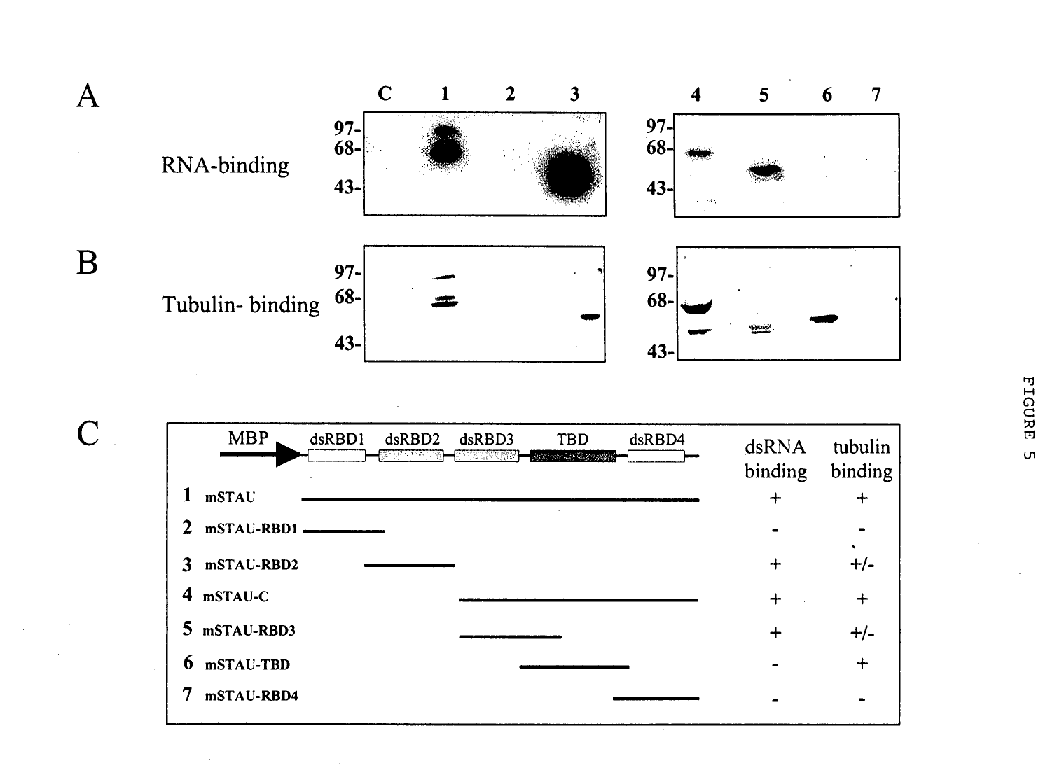

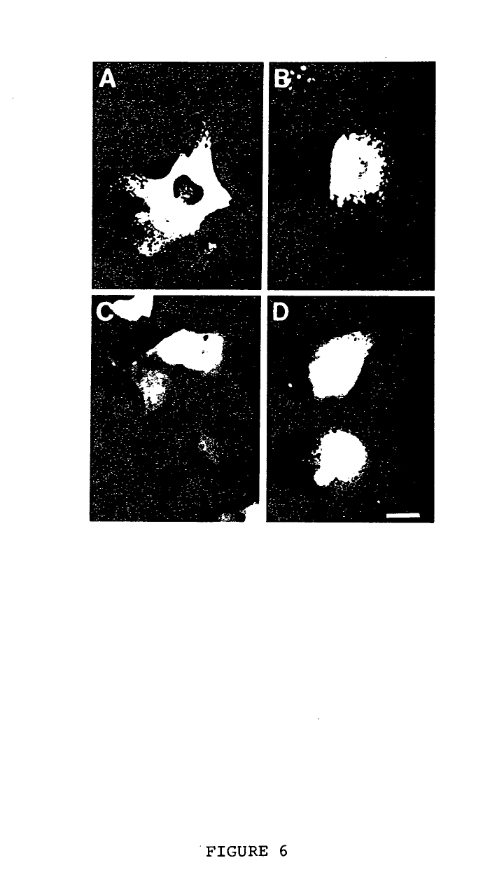

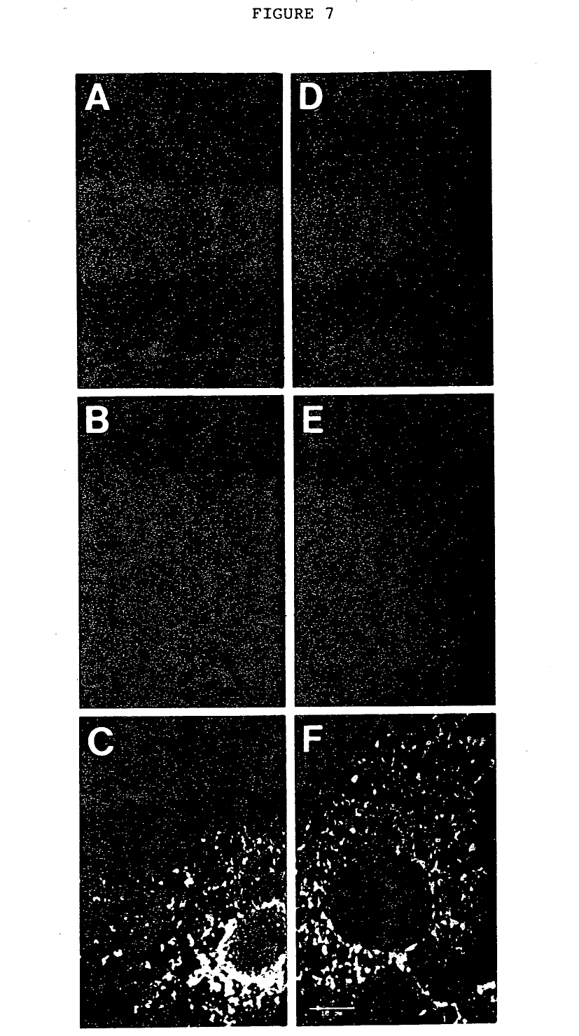

Interestingly, the pattern of localization of Stau resembles that of the endoplasmic reticulum. To test a putative localization of Stau to the ER, mammalian cells were transfected with a cDNA coding for a fusion protein in which a HA-tag was introduced at the C-terminal end of the short hStau protein. The cells were then double-labeled transfected with anti-HA, to recognize hStau, and with anti-calreticulin or anti-calnexin, two markers of the ER. Using a confocal microscope, it was shown that hStau completely co-localizes with anti-calreticulin, although HA-staining appears to be absent in some parts of the ER, in particular around the nucleus (FIG. 7A-C). To confirm these results, the co-localization of Staufen and calnexin, a specific marker for the RER (Hochstenback et al. (1992), PNAS 89, 4734-4738) (FIG. 7D-F) was examined. The patterns of staining obtained with anti-hStau and anti-calnexin were identical, demonstrating that hStau co-localizes exclusively with the RER.

### EXAMPLE 17

### Implication of Staufen in mRNA Transport and Localization

The transport and localization of specific mRNAs have important functions in cell physiology. For example, mRNA targeting plays a key role in the formation of cytoskeletal filaments and in the establishment of morphogenetic gradients (St Johnston, 1995, supra). However, the nature of the ribonucleoprotein complexes as well as the mechanisms involved in these processes are still largely uncharacterized. Herein, a novel RNA-binding protein which localizes to the rough endoplasmic reticulum in mammalian cells has been described. Although its precise role is still unclear, its biochemical and molecular properties strongly suggest that it is involved in mRNA transport and/or localization. Consistent with such a role, we recently demonstrated that hStau is involved in HIV-1 genomic RNA encapsidation

Similarly, a mammalian Staufen homologue was shown to be involved in the polarized transport of mRNAs in hippocampal neurons (Kiebler et al J. Neurosci. 1999 Jan 1 ;19(1):288-97).

### EXAMPLE 18

### Structure/Function of Staufen

As is the case for all members of the dsRNA-binding protein family (St Johnston, 1995, supra), it was observed that mammalian Staufen can bind any dsRNA or RNAs forming extensive secondary structures in vitro, regardless of its primary sequence, as well as RNA/DNA hybrids. The latter adopt a conformation that is more closely related to that of dsRNA than dsDNA, which probably explains why they can bind to Staufen. The fact that the full-length Stau protein, as observed with single dsRBD, binds to any dsRNA in vitro, suggests that the correspondence between the position of the dsRNA-binding domains and the arrangement of double-stranded stems in the folded RNAs may not be sufficient for specificity; post-translational modifications and/or essential co-factors capable of forming complex ribonucleoprotein structures along with mRNA molecules, could be necessary to discriminate between different RNA secondary structures. Packaging of mRNAs into ribonucleoprotein complexes (Ainger et al. (1993), J. Cell. Biol. 123, 431-441; Ferrandon et al., 1994, supra; Forristall et al. (1995), Development 121,201-208; Knowles et al. (1996), J. Neurosci. 16, 7812-7820), the intermolecular dimerization of the localization signal of bicoid mRNA (Ferrandon et al. (1997), EMBO J. 16, 1751-1758) and the involvement of untranslatable hnRNAs in mRNA transport (Tiedge et al. (1991), PNAS 88,2093-2097; Tiedge et al. (1993), J. Neurosci. 13, 4214-4219; Kloc et al. (1994), Science 265, 1101-1103), are consistent with this interpretation. Until now, specific mRNA/Staufen interactions were only shown in vivo after injection of different RNAs into Drosophila embryos, but the mechanisms underlying the specificity are not known (Ferrandon et al., 1994, supra). Since specific RNA binding cannot be obtained in vitro, it precludes the use of classic techniques to isolate and identify relevant RNAs which would bind Staufen in vivo. Cross-linking of mRNA to Staufen in vivo, and isolation of the resulting complexes will be necessary to identify the nature of bound RNAs.

Regardless of their limitations, the in vitro assays did allow a mapping of the molecular determinants which are necessary and sufficient to bind RNAs. The presence of two functional domains in the mammalian Stau contrasts with what has been reported for other members of the dsRNA-binding protein family, which contain multiple full-length dsRBDs, but only one that is biochemically functional (Gatignol et al.-(1993), Mol. Cell. Biol. 13, 2193-2202; McCormack et al. (1994), Virology 198, 92-99; Schmedt et al. (1995), J. Mol. Biol. 249, 29-44; Krovat et al. (1996), A. J. Biol. Chem. 271, 28112-28119). Interestingly, full-length dsRBDs incapable to bind dsRNA by themselves can do so when joined to another inactive full-length domain, suggesting that multiple domains present in a given protein exhibit cooperative binding effect (Schmedt et al., 1995, supra; Krovat et al., 1996, supra). Whether the two mStau dsRNA-binding domains exhibit similar or different affinities is not yet clear. However, the identification of the molecular determinants of Staufen necessary and sufficient for RNA binding open the way to a wide variety of utilities. Non-limiting examples include viral therapy and prevention, targeting of molecules (comprising Staufen□s incorporation domain) into virions and gene therapy. In this respect, the PCT publication of Cohen et al. WO 96/07741 is of relevance, as it identified a new means for targeting molecules into HIV virions. The teachings of WO 96/07741, including vpr/vpr fusion proteins, vpr/vpr recombinant proteins and nucleic acid molecules encoding same can be applied to the present invention, now that Staufen has been identified as a RNA-virus targeting protein and more particularly as a HIV targeting protein.

Tubulin-binding domain was mapped to a region which is similar to a microtubule-binding domain of MAP1B. Although this region can efficiently bind tubulin in vitro, it is not sufficient to bring a TBD/GFP fusion protein to the microtubule network. Binding of Stau to microtubules in vivo may involve more than one molecular determinant or the proper localization and folding of the TBD in the full-length protein. Indeed, in our in vitro assay, the fusion protein which contains the C-terminal region in addition to the TBD binds tubulin more efficiently than does the TBD, alone, suggesting that this region may be necessary for binding to microtubules in vivo. Interestingly, the corresponding region of the Drosophila Staufen protein was shown to bind inscutable (Li et al., 1997, supra), a protein with ankyrin domains which is believed to associate with the cytoskeleton (Kraut et al. (1996), Dev. Biol. 174, 66-81), suggesting that corresponding regions of the mammalian and Drosophila proteins may have functional similarities. The characterization of the mammalian Staufen can therefore provide a guidance for a broadering of the present teachings to lower eukaryotic Staufen such as that of Drosophila and as of C. elegans.

Alternatively, binding may be weak and/or transitory in vivo, for example during the early steps of mRNA recruitment, during mRNA transport and/or at mitosis, as reported in Drosophila (Ferrandon et al., 1994, supra; Pokrywka et al: (1995), Dev. Biol. 167, 363-370; St Johnston, 1995, supra). These steps may be difficult to observe by immunofluorescence (Ferrandon et al., 1994, supra), and/or be masked by the anchoring of the protein to the RER. These steps may nevertheless be necessary to allow efficient and flexible transport of RNA along the cytoskeleton. In Drosophila, there is no evidence that Staufen directly binds to the microtubule network, although Staufen-dependent mRNA transport was shown to rely on this structure (Pokrywka et al., 1995, supra; St-Johnston, 1995, supra). A similar conclusion was reached when binding of MAP1B to the microtubule network was studied (Zauner et al., 1992, supra), suggesting that weak binding to the cytoskeleton may be a characteristic of proteins containing this type of tubulin-binding domain.

The present teachings demonstrate that Stau is anchored to the RER and that the putative TBD is not involved in this function. Indeed, preliminary results suggest that the binding of Stau to RER is carried out by one of the RNA-binding domains (data not shown). Similar domains in other members of the dsRNA-binding proteins were previously shown to be involved in protein dimerization and/or in protein/protein interactions (Cosentino et al. (1995), PNAS 92, 9445-9449; Benkirane et al. (1997), EMBO J. 16, 611-624). This also suggests that different Stau molecular determinants are necessary for binding to tubulin and anchoring to the RER. This is consistent with previous observations in Xenopus and Drosophila that demonstrated that mRNA localization was likely to occur via successive steps involving different elements of the cytoskeleton and overlapping molecular determinants (St Johnston, 1995, supra).

### EXAMPLE 19

### Localization of Staufen to the RER

When expressed in mammalian cells, Stau isoforms show a tubulovesicular pattern of localization which is found more abundantly in the perinuclear region. Stau is the first RNA-binding protein shown to be associated with the RER in mammals. No signal peptide or putative hydrophobic transmembrane domains are present in either the long or short Staufen proteins, indicating that they are cytosolic proteins and not residents of the RER and that their association to the RER is likely to reflect their mRNA transport function. Two recent papers also suggest that mRNA transport may be linked to the endoplasmic reticulum or ER-like structures. In Xenopus oocytes, vera, a Vg1 mRNA binding protein, was shown to co-sediment with TRAPa, a protein associated with the protein translocation machinery of the ER. However, in contrast to Stau, vera/Vg1 complexes were found associated only with a small subdomain of the ER, which was of the smooth variety (Deshler et al. 1997, Science 276, 1128-1131). Similarly, in Drosophila, at least some steps in mRNA transport in nurse cells and oocytes seem to occur within ER-like cisternae (Wilsch-Bräuninger et al. (1997), J. Cell Biol. 139, 817-829). As observed for the Vg1 mRNA/SER interaction in Xenopus, this structure seems to exclude most ribosomes, suggesting that translation is not the major function of these associations.

Hstau and mStau represent new members of a large family of proteins involved in the transport and/or localization of mRNAs to different sub-cellular compartments and/or organelles. Stau, TRBP/Xlrbpa and Spnr were shown to co-localize with RER (see above), with ribosomes and heterogenous nuclear RNPs (Eckmann et al. (1997), J. Cell Biol. 138, 239-253), and with the microtubular array of spermatids (Eckmann et al. (1997), J. Cell Biol. 138, 239-253), respectively. The present results strongly suggest that Staufen/mRNA ribonucleoprotein complexes are transported along the microtubule network and then anchored to the RER. It is well known that the ER is associated with the microtubule cytoskeleton (Terasaki et al. (1986), J. Cell Biol. 103, 1557-1568). Therefore, a transient interaction between microtubules and Stau may facilitate the localization of Stau and the targeting of mRNA to the RER. One of the roles of Stau might be to transport and localize specific mRNAs to the RER, such as those coding for secreted or membrane proteins which have to be translocated to the RER. This would bring them in proximity to the signal recognition particles (SRP) and RER, thus facilitating translation and translocation. The presence of Stau in cell processes, in association with ER structures, may represent a first clue to understanding the role of many mRNAs coding for neuropeptides, receptors or ion channels which were found to be localized in neuronal processes (Steward, 1997, supra). Stau may facilitate the transport of mRNAs to cell processes to ensure efficient local translation and translocation. In addition, the presence of multiple Staufen-like proteins in mammals creates the possibility that different members of the family could target sub-classes of mRNAs to different sub-domains of the ER. This phenomenon has been described before, and is thought to be the first step in the differential targeting of proteins in polarized cells (Okita et al. (1994), TIBS 4, 91-96).

The possibility that Staufen plays additional roles in mammals is not excluded; Stau may first be linked to the RER for storage, then a subset of molecules may be recruited by specific mRNAs and/or cofactors to form ribonucleoprotein complexes that will be transported along microtubules toward their final destination. Consistent with this possibility is the presence of large amounts of Stau in the perinuclear region, where it may await the nucleo-cytoplasmic transport of mRNAs. Alternatively, Stau may play key roles in the regulation of translation of localized mRNAs. The fact that Drosophila Staufen is essential for the translation of oskar mRNA, once it is localized at the posterior pole, is consistent with this hypothesis (Kim-Ha et al. (1995), Cell 81, 403-412). Characterization of mRNAs and putative co-factors which bind to Staufen will be necessary to understand the process.

In vertebrates, the mechanisms which underly the transport of mRNAs have not yet been deciphered. Characterization of the RNAs and proteins involved in transport and localization is particularly important since understanding the mechanisms responsible for the transport of mRNAs is fundamental for learning more on the development of polarity in cells, both during mammalian development and in somatic cells, at a time where RNA-based gene therapy is being considered as a possible approach to cure different disorders.

The present invention therefore opens the way to a development of better strategies for RNA-based gene therapy.

### EXAMPLE 20

### Staufen is Incorporated into HIV-1 Virions

In order to assess the functional significance of the dsRNA-binding activity of Staufen in mammalian cells, the possibility of its binding to the TAR sequence in the HIV-1 RNA leader was investigated (FIG. 8A). Its association with HIV-1 was further investigated by determining whether hStau was incorporated into HIV-1 particles, a possible result of its double-stranded RNA binding capacity. Indeed, using a polyclonal antiserum generated to highly purified recombinant hStau, the corresponding 55 and 63 kDa species (Wickham et al. (1996), Genomics 36:527) of Staufen were identified in purified viral preparations of laboratory strains of HIV-1 HxBc2 (HxBru, HxBH10) and pNL4.3, and in vesicular stomatitis virus G (VSVG) envelope pseudotyped HIV-1 particles (data not shown and FIG. 8) generated in human T lymphocyte (MT4 and Jurkat) or epithelial (293T) cell lines (data not shown).

To further substantiate hStau virion incorporation, sucrose gradient analyses were performed. First, microfiltered and ultracentrifuged HxBru virus was prepared in 293T cells. This cell type produces negligible amounts of contaminating microvesicles that contain cellular proteins (Fortin, J. F. et al. (1998), J. Virol. 72;2105). The virus was fractionated in a 20-60% sucrose gradient, and the presence of hStau in each fraction was evaluated by Western blot analysis. hStau was found to cosediment with reverse transcriptase (RT) activity, strongly indicating incorporation or strong association with viral particles (FIG. 8B). To further support virion incorporation, a subtilisin protease assay was performed on virus preparations (Ott et al. (1995), AIDS Res. Hum. Retroviruses 11, 1003). While envelope glycoprotein gp120 was completely degraded as expected after subtilisin treatment, viral proteins p24 and p17 remained in large part protected since they are found within the virus (FIG. 8C). hStau also remained intact (FIG. 8C), though there appeared to be some degradation by subtilisin treatment. This same phenomenon was recently observed in virus generated in H9 and CEM cells where an actin isoform was shown to be incorporated within HIV-1 particles while some of the protein was also sensitive to subtilisin treatment (Ott et al. (1996) J. Virol. 70, 7734).

Incorporation of hStau in two T-tropic viral clinical isolates minimally passaged in MT4 cells, and in three other retroviruses, HIV-2, murine leukemia virus. (MLV) and Casitas brain ecotropic MLV (CasBr; Bergeron et al. (1991), J. Virol. 65:7) was then examined. All of these vector viruses incorporated hStau (FIG. 8D) suggesting a common functional role. Of note, hStau was also shown to be incorporated into a non retrovirus RNA virus, Reovirus (data not shown). Purified cell-free preparations of the DNA viruses, adenovirus, Epstein Barr virus (EBV) and human herpesvirus 6 (HHV-6) did not contain hStau. The presence of hStau was evaluated in concentrated cell-free and cesium chloride-banded preparations of Adenovirus (kindly provided by Dr. Bernard Massie, Biotechnology Research Institute, Montreal, Quebec), EBV and HHV-6 (both kindly provided by Drs. Ali Ahmad and José. Menezes, Department of Microbiology and Immunology, University of Montreal). hStau was assessed by Western blot analysis: there were no detectable bands corresponding to hStau in up to 20×10⁹ viral particles.

While hStau is incorporated into virions, the dsRNA- and TAR RNA-binding proteins TAR RNA-binding protein (TRBP), dsRNA-activated protein kinase (PKR) and Tat, are not detectable in purified preparations of HIV-1 (FIG. 8E). Taken together, these data show that the TAR-binding activity is not sufficient to enable virion incorporation.

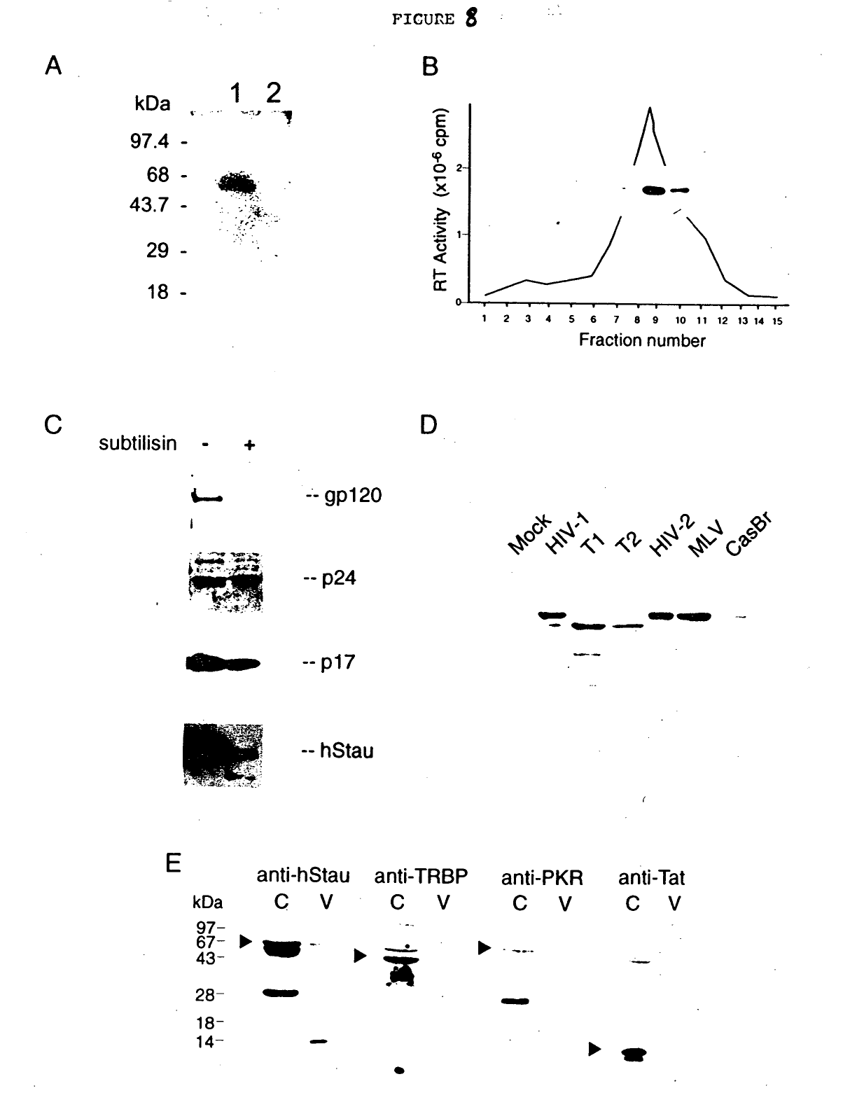

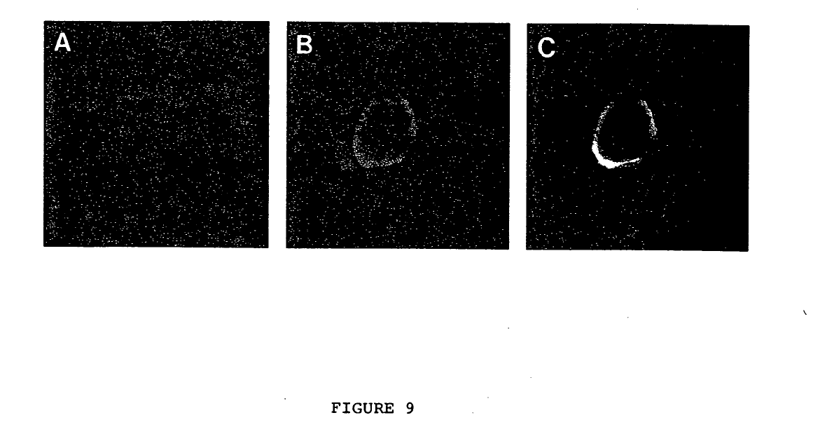

Confocal laser scanning microscopy was employed to determine the precise localization of hStau in HIV-1-producing cells. pNL4.3 and a hemagglutinin (HA)-tagged hStau were coexpressed in 293T cells and p24 and hStau were visualized by Texas Red- and fluorescein-conjugated secondary antibodies, respectively, in indirect immunofluorecence analyses (FIG. 9). hStau showed a diffuse cytoplasmic staining (Wickham et al., 1996, supra) and a large proportion of hStau was found to be colocalized with p24 antigen at the cell periphery (FIG. 9C). This colocalization is suggestive that hStau is present at sites of virus assembly, consistent with its presence in virions.

### EXAMPLE 21

### Characterization of Molecular Determinants, Involved in Staufen Incorporation into HIV-1

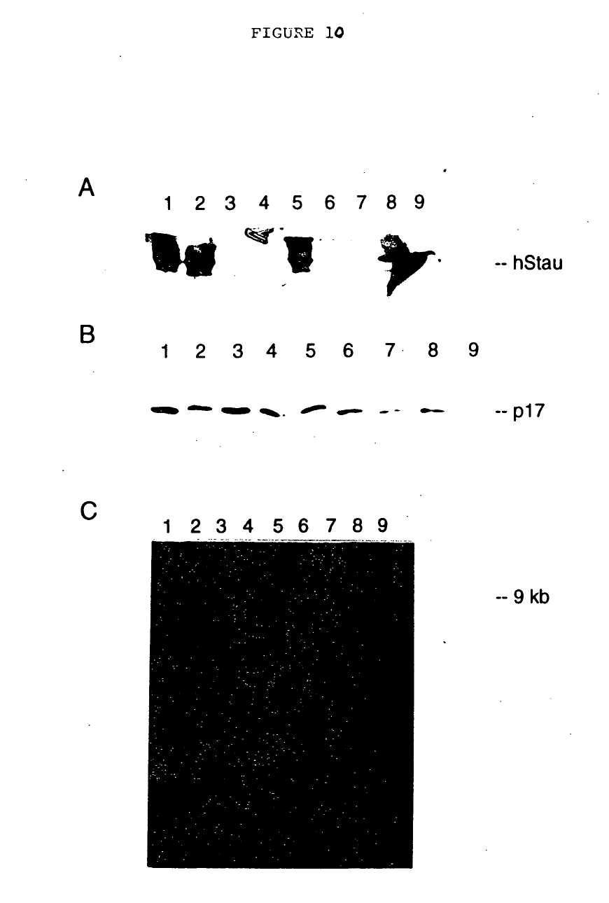

On the basis of hStau TAR RNA-binding and its virion incorporation, a role for hStau in virus assembly was investigated. It was therefore attempted to correlate genomic RNA encapsidation with hStau incorporation in HIV-1. Transfection of wildtype provirus DNAs yields virus particles containing comparable amounts of hStau (FIG. 10, lanes 1 & 2). Genomic RNA encapsidation in HIV-1 is primarily mediated through the association of the packaging (psi) domain in the 5′ leader sequence with the nucleocapsid (NC) protein (McBride et al. (1997), J. Virol.71, 4544; Lever et al. (1989), J. Virol. 63,4085; Berkowitz, R. D. et al. (1995), Virology 212,718; Poon et al. (1998), J. Virol. 72,1983). Therefore, an HIV-1 molecular clone HxBru in which the ²⁸Cys and ⁴⁹Cys of NC were mutated to Ser (²⁸C/⁴⁹C-S; 14) was initially tested. It was found that hStau incorporation was drastically reduced in these virus preparations (cf. FIG. 10, lane 3). Several other HIV-1 proviruses with NC mutations and deletions (Huang et al. (1997), J. Virol. 71, 4378), and a psi domain deletion mutant (Miele et al., 1996, supra) were then tested, most of which generate noninfectious virus particles that are significantly impaired in RNA encapsidation. With the exception of the ³⁶C/³⁹C-S NC mutant, transfection of all NC and psimutant DNA proviral constructs generated virus particles that contained negligible amounts of hStau. Genomic RNA encapsidation was assessed in Northern blots and these analyses revealed that the psi and NC mutant constructs yielded virus with drastically reduced levels of genomic 9 kilobase pair (kb) RNA. In the ³⁶C ³⁹C-S NC mutant virus preparation (FIG. 10, lane 5) hStau is present at approximately wildtype levels, and at the same time near wildtype levels of genomic RNA encapsidation are observed, consistent with several earlier observations (Mizuno et al. (1996), AIDS Res. Hum. Retrov. 12:793, Gorelicket al. (1990), J. Virol. 64:3207; Gorelick et al. (1993), J. Virol. 67:4027; Dannuil et al. (1994), EMBO J. 13:1525). hStau incorporation into HIV-1 particles is thus strongly correlated with genomic RNA encapsidation. Consequently, hStau may indeed sort viral RNAs into a vicinity of an infected cell where Gag proteins are present, during assembly of virus particles. Alternatively, the data presented herein suggest that hStau incorporation is mediated through both the psi and NC domains; and with the recent structural characterization of NC-psi binding (De Guzman et al. (1998), Science 279, 384) it will be interesting to determine whether hStau is necessary for this conserved and critical association.

### EXAMPLE 22

### Expression of Staufen from HIV-1 Chimeric Proviruses

For the construction of chimeric proviruses Staufen-HA cDNA was PCR-amplified from pcDNA3-RSV-hStau-HA (44) using the Pfu polymerase (Stratagene) and sense (5′-GATGCTCGAG ATGAAACTTG GAAAAAAACC-3′, SEQ ID NO. 46) and antisense (5′-CACATCTAGA TCATTTATTC AGCGGCCGCA CtGAGCAGCGT-3′, SEQ ID NO.47) oligonucleotides. Resulting PCR products were digested with Xho I and Xba I and cloned in pNL4-3/PKR (Benkirane et al. (1997), EMBO J. 16:611-624) to replace the XhoI/XbaI pkrfragment in the nef open reading frame. The same approach was used to introduce two Staufen mutants in the nef open reading frame: a full-length Staufen protein with a point mutation in dsRBD3 (StaufenF1³⁵A) and a C-terminal truncated mutant (dsRBD2-4) lacking the tubulin binding domain (TBD) and dsRBD5. HxBRU provirus (Yao et al. (1998), J. Virol. 72:4686-4693) was used in the siRNA experiments described below.

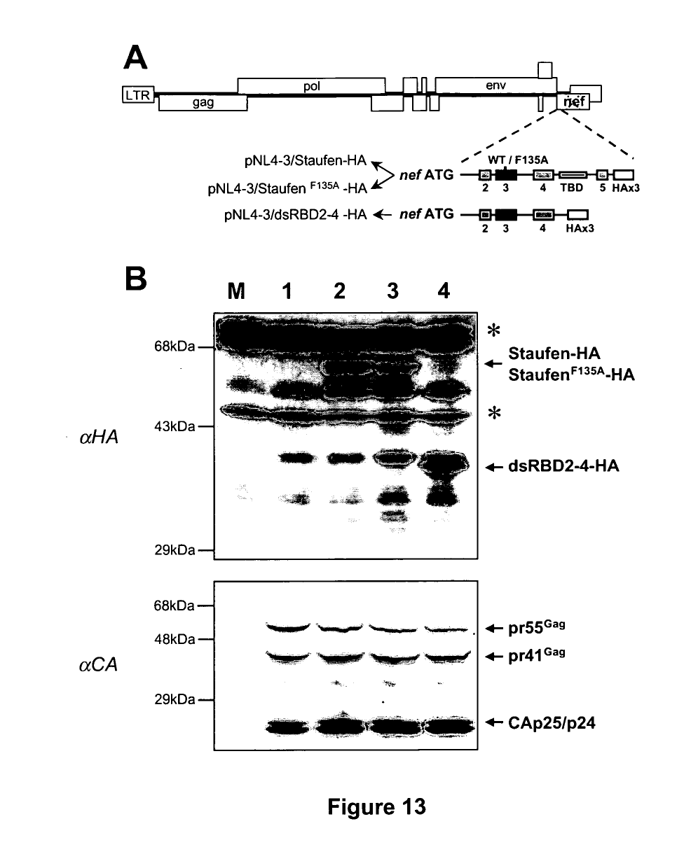

These HIV-1 chimeric proviruses, in which Staufen cDNA was inserted into the nef open reading frame (FIG. 13A), were constructed in order to characterize a putative interaction between Staufen and HIV-1 proteins/RNA. In this context, Staufen should be expressed early during HIV-1 expression, since Nef is amongst the first viral proteins to be expressed. We also prepared two additional chimeric proviruses containing either the full-length Staufen protein with a point mutation in dsRBD3 that abolishes its RNA binding capacity (StaufenF^{135A}), or a C-terminal truncated mutant (dsRBD2-4) lacking the tubulin binding domain (TBD) and the dsRBD5. To verify Staufen expression in this proviral context, expression levels were analyzed by Western blotting of extracts of transfected 293T cells. As shown in FIG. 13B, Staufen proteins were all expressed at comparable levels. In addition, Staufen expression in the context of the provirus did not affect Gag expression levels or Gag processing since the same levels of pr55^{Gag}, CA and CAp25 were observed (FIG. 13B).

### EXAMPLE 23

### Staufen and HIV-1 pr55^{GAG} Cofractionate on Sucrose Gradient

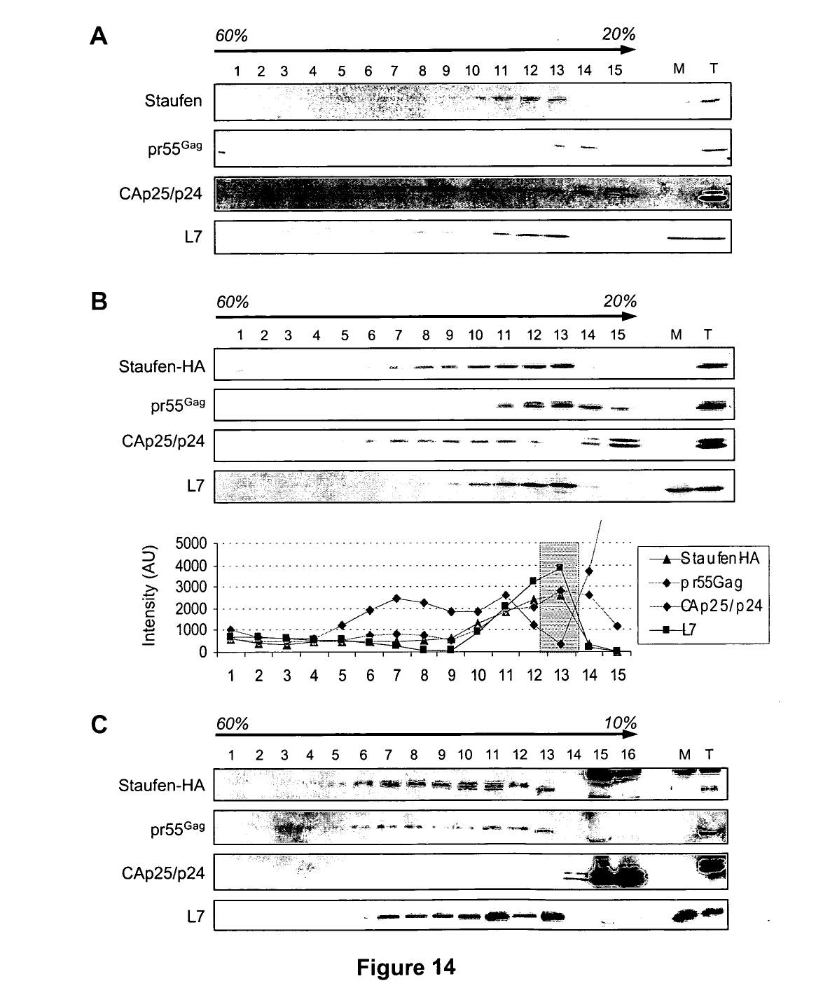

The subcellular distributions of Staufen and HIV-1 Gag during HIV-1 replication were studied. These studies showed that Staufen and pr55^{Gag} partially colocalized in the cytoplasm of HIV-1/Staufen expressing cells (data not shown). Sucrose gradient sedimentation assays were performed in order to determine the relationships between cytosolic Staufen and HIV-1 pr55Ga9 or CA. This technique allows for the separation of cellular compartment/organelles according to their buoyant density. Cytoplasmic cell extracts were prepared from pNL4-3- and pNL4-3/Staufen-HA-expressing cells and separated on sucrose gradients (20-60% w/v in Panels A and B of FIG. 14). Each fraction was analyzed by Western blotting for the presence of Staufen and HIV-1 proteins.

In detail, 293T cells were homogenized for 1 min in 12.5% sucrose, 10 mM HEPES pH 7.3, 1 mM EDTA and then centrifuged at 1000×g for 5 minutes. The supernatant was layered onto a continuous 20 to 60% (w/v) sucrose gradient (in 10 mM HEPES pH 7.3, 1 mM MgCl₂) as reported (Luo, M. et al. (2002), Biochem J. 365:817-824). In other experiments to increase stringency, cells were homogenized in a lysis buffer containing detergent (100 mM NaCl, 10 mM Tris, 1 mM EDTA, 0.5% NP-40, pH 7.3) and layered onto a 10 ml continuous 10 to 60% (w/v) sucrose gradient. Both gradients were centrifuged at 31,000×g for 2.5 h in an SW41 rotor (Beckmam-Coulter). Fractions were collected from the top and analyzed by Western blotting for Staufen (using mouse monoclonal anti-HA or anti-Staufen antibodies), CA, MA (from Dr. Spearman, NIH AIDS Reference and Reagent Program), and ribosomal L7 (Novus Biologicals).

In cells that express pNL4-3 only, endogenous Staufen was found in fractions 10 to 13 with a peak in fraction 13. Ribosomal protein L7, a marker of whole ribosomes, was also found in these Staufen fractions (FIG. 14A) as shown previously (Luo et al., 2002, supra). When anti-CA was used to identify Gag proteins, unprocessed pr55^{Gag}, like Staufen, was principally found in fractions 11 to 14 with a peak in fraction 13 (indicated in the shaded box under FIG. 14B). In contrast, the processed forms of pr55^{Gag}, CAp24/p25 sedimented mainly in fractions 6 to 12, likely corresponding to budding viruses. Approximately 50% of the total CA was detected in the low density fraction 15, likely representing lysed viruses. Identical results were obtained for the distribution of Staufen-HA in cells expressing pNL4.3/Staufen-HA (FIG. 14B) such that Staufen co-fractionated with pr55^{Gag} and not with the processed forms of pr55^{Gag}. This set of experiments demonstrates that while the distribution of Staufen and pr55^{Gag} were not identical, both proteins cosedimented in sucrose gradients and peaked in the same fraction. It also shows that the expression of Staufen in the context of pNL4-3/Staufen does not affect the cytoplasmic distribution of HIV-1 proteins, and moreover, HIV-1 gene expression does not have any apparent effects on that of Staufen.

To further characterize these complexes, these sucrose gradient analyses were repeated in the presence of non-ionic detergent (FIG. 14C; using a 10-60% sucrose gradient). This treatment disrupts weak interactions and releases membrane-associated complexes. Under these conditions, Staufen and pr55^{Gag} co-sedimented with a similar profile in intermediate density fractions 7-13. Because a 10-60% gradient was used in this latter analysis, we expected that the sedimentation profiles of Staufen and pr55^{Gag} profiles be slightly different, which is what we observed. The data show that detergent treatment did not significantly change the sedimentation profile of either Staufen or pr55^{Gag}. These proteins sedimented in fractions 7-13 (34.5-24% sucrose) after detergent treatment (FIG. 14C) or mainly in fractions 10-13 (34.5-25% sucrose) without detergent (FIG. 14A or 2B). In contrast, the processed forms of Gag such as CAp25/p24 and MA (not shown) were no longer present in high density fractions (6-12 in FIG. 14A-B) but were found at the top of the gradient (mainly in fractions 14 and 15) following detergent treatment, likely corresponding to detergent-solubilized viral particles. Therefore, Staufen and pr55^{Gag} are found in detergent-resistant complexes (DRC) consistent with previous Gag data (Lee et al. (1999), J. Virol. 73:5654-5662, Lee et al. (1998), Virology 243:78-93). When cell extracts were treated with trypsin before gradient analysis, neither Staufen nor pr55^{Gag} was detectable in sucrose gradients indicating that both proteins are sensitive to trypsin digestion and are mainly in a cytosolic compartment (data not shown). In contrast, CAp25/p24 levels and distribution were not affected since they are most likely protected from trypsin by their association to membranes. The herein presented results now show that Staufen co-fractionates with pr55 Gag and is likely part of the same HIV-1 DRC, a complex that was previously shown to mainly contain pr55^{Gag} and to correspond to cytoplasmic virion assembly intermediates (Lee et al., 1999, supra, Lee et al., 1998, supra).

### EXAMPLE 24

### Staufen Interacts with HIV-1 pr55^{GAG} in a RNA-Independent Matter

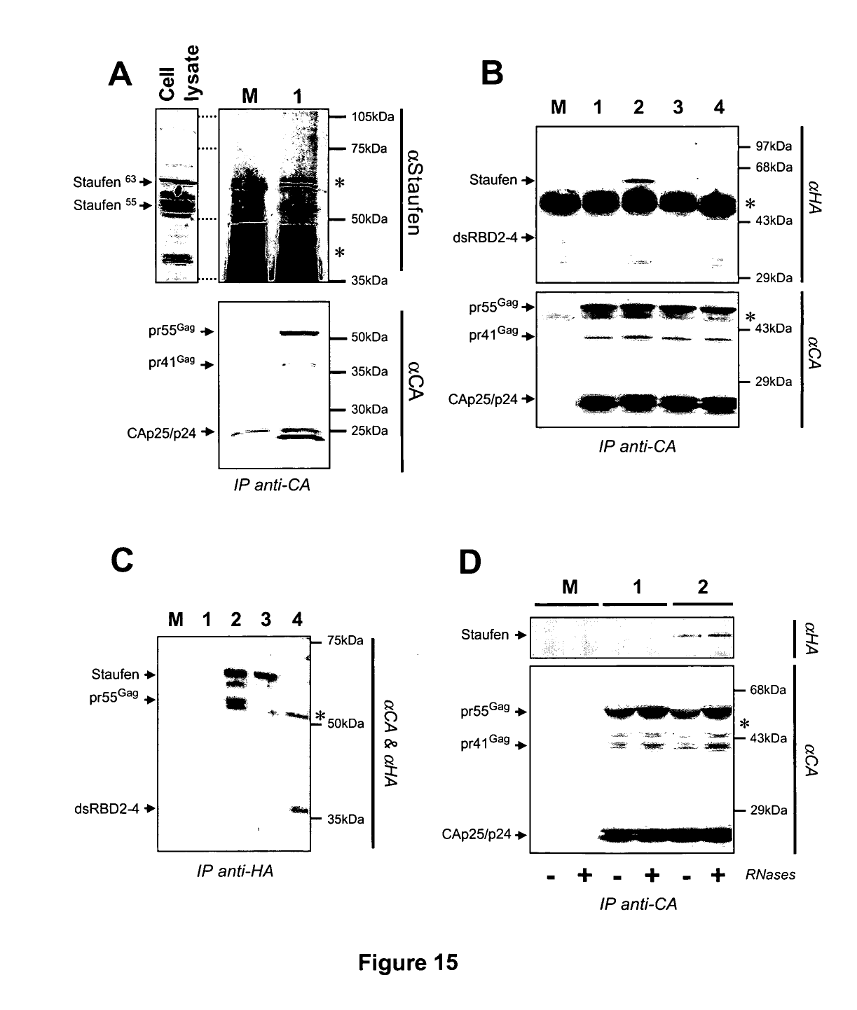

In preliminary studies, yeast two hybrid analyses revealed that full-length Staufen and pr55^{Gag} interacted (Mouland et al. (2000), supra) to a level similar to that found for pr55^{Gag}/pr55^{Gag} (Bachand, F. et al. (1999), J. Biol. Chem. 274:9083-91). Immunofluorescence results also suggested that at least a proportion of Staufen and pr55^{Gag} are in close proximity and could be components of the same HIV-1 assembly complex (data not shown). To clearly address this possibility, it was first determined whether endogenous Staufen interacted with pr55^{Gag} during proviral expression. For this, cells were mock transfected or transfected with pNL4-3 alone. Extracts from these cells were immunoprecipitated using a monoclonal anti-CA antibody. A cell extract was analyzed for endogenous Staufen prior to immunoprecipitation using a rabbit polyclonal antibody used previously (Mouland et al., 2000, supra) FIG. 15A, top-left panel). This antibody reacts with two isoforms of Staufen [(55 and 63 kDa (Mouland et al. (2000), supra)]. pr55^{Gag} was then immunoprecipitated from equal quantities of extracts and the immunoprecipitates were analyzed for the presence of endogenous Staufen protein (FIG. 15A, top-right panel), and immunoprecipitated Gag products (FIG. 15A, lower panel). Staufen was found to be present in the HIV-1 Gag immunoprecipitates during HIV-1 expression, but not in the immunoprecipitate from mock-transfected cell lysates. There was a specific immunoprecipitation with the 55 kDa Staufen isoform (FIG. 15A, top-right panel). A 63-65 kDa protein signal was obtained in both conditions and is not related to the 63 kDa Staufen isoform. These results thus suggest that endogenous Staufen is physically recruited to pr55^{Gag}-containing complexes during proviral gene expression.

In order to map the determinants in the Staufen protein that are important for this interaction, 293T cells were transfected with the chimeric Staufen proviruses shown in FIG. 13 followed by immunoprecipitation/Western analysis. Cell lysates were immunoprecipitated with anti-CA antiserum, and Staufen-HA was identified in the immunoprecipitates by Western blot. From cells transfected with pNL4-3/Staufen-HA provirus, a 60 kDa band corresponding to Staufen-HA was detected using an anti-HA antibody in the Gag immunoprecipitate, whereas Staufen was not detected in Gag immunoprecipitates from pNL4-3/StaufenF”³⁵A-HA- or dsRBD2-4-HA-expressing cells (FIG. 15B). An approximately equal amount of Gag (pr55^{Gag}, pr41^{Gag}, CAp24/p25) was immunoprecipitated in all conditions. These results demonstrate that Staufen's interaction with pr55^{Gag} in HIV-1-expressing cells requires contributions from both an intact dsRBD3 and C-terminal domain.

In the reverse experiment, cells were transfected with pNL4-3 or the pNL4-3/Staufen proviruses and Staufen-HA was immunoprecipitated using anti-HA. Immunoprecipitated proteins were then analyzed by Western blot analysis using monoclonal anti-CA. Anti-HA was included to identify Staufen-HA proteins at the same time. This analysis shows that pr55^{Gag} co-immunoprecipitates with wildtype Staufen-HA but not with either of the Staufen mutants (FIG. 15C). We were not able to detect processed forms of pr55^{Gag}, CA and NC, in the immunoprecipitates (data not shown). These results are consistent with the gradient analysis presented herein which show that these do not co-fractionate with Staufen (FIG. 14A-B). These data strongly suggest that Staufen preferentially interacts with the Gag precursor, pr55^{Gag}.

Since a single point mutation in Staufen's dsRBD3 abolishes both its RNA-binding activity (29) and the Staufen/pr55^{Gag} interaction (FIG. 15B), we investigated whether RNA contributes to this interaction, as it does for the pr55^{Gag}/EF-1α, pr55^{Gag}/pr55^{Gag} and pr55^{Gag}/Gag-Pol (or pr160^{Gag/Pol}) interactions (Burniston, M. T. et al. (1999), J. Virol. 73:8527-40, Cimarelli, A. et al. (1999), J. Virol. 73:5388-401, Cimarelli, A. et al. (2000), J. Virol. 74:3046-57, Khorchid, A. et al. (2002), J. Virol. 76:4131-7). Cell lysates were mock-digested or digested with RNases A and T1 prior to immunoprecipitation with anti-CA antiserum. As shown in FIG. 15D, Staufen remained associated with Gag-containing complexes in the presence of RNase, demonstrating that this interaction is maintained even if the cellular RNA is removed. However, we cannot exclude the possibility that bridging RNA may be protected from RNase digestion by the Staufen/pr55^{Gag} complex using this assay and these results, while reproducible, should be interpreted with care.

### EXAMPLE 25

### Staufen/pr55^{GAG} Interaction Requires the HIV-1 NC Domain

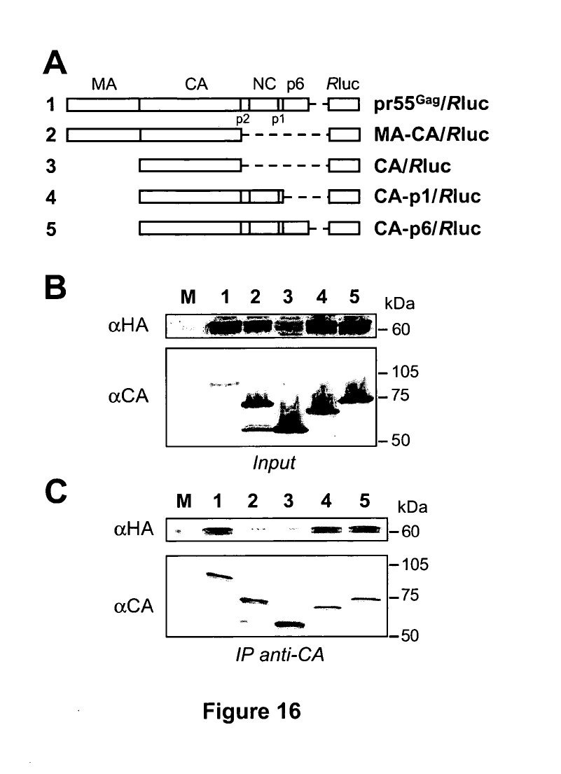

Co-immunoprecipitation assays were also used to map the pr55^{Gag} domain that interacts with Staufen. Full-length pr55^{Gag} as well as four deletion mutants containing different Gag subdomains (MA-CA, CA, CA-p1, CA-p6) were fused in-frame with Renilla reniformis luciferase (Rluc) (FIG. 16A).

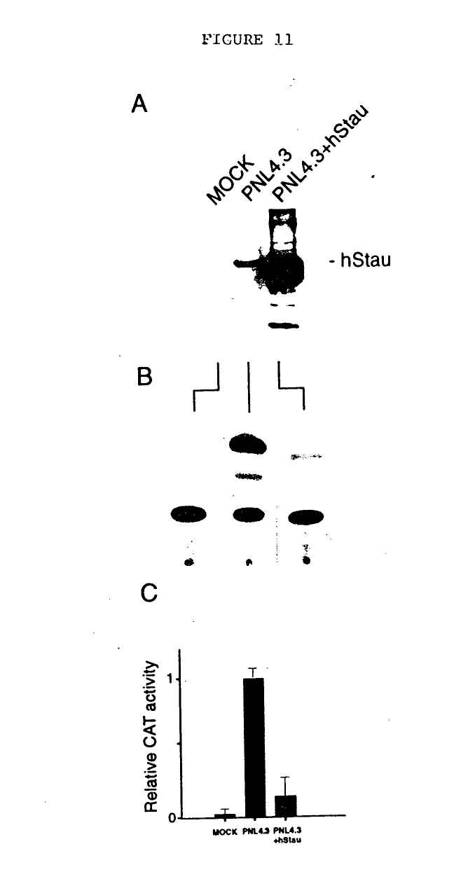

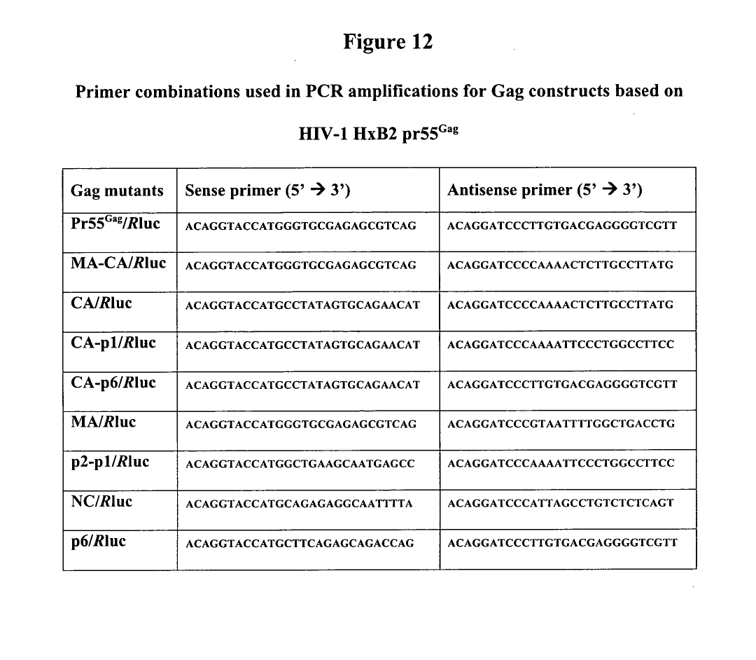

The construction of pr55^{GAG} mutants was performed as follows: DNA fragments encoding full-length or different parts of HxB2 Gag polyprotein were PCR-amplified by the Pfu Turbo DNA polymerase (Stratagene) using the Rev-independent Gag expressor pCMV55M1-10plasmid (Schneider, R. et al. (1997), J. Virol. 71:4892-903) as template. Primer pairs used in the PCR reactions are summarized in FIG. 12. PCR products were digested by KpnI and BamHI and inserted in the KpnII/BamHI cloning sites of pRluc-N1(h) (Packard BioScience/PerkinElmer Life Sciences) in frame with the Renilla reniformis luciferase coding sequence (Angers, S. et al. (2000), Proc Natl Acad Sci USA. 97:3684-9). To construct pCMV-Staufen/YFP, pcDNA3-RSV-Staufen-HA (Wickham et al. (1999), Mol. Cell Biol. 19:2220-2230) was digested with NotI, treated with the Klenow fragment of DNA polymerase to blunt the extremities and digested with HindIII. The resulting fragment was then inserted in the HindIII/SmaI sites of pCMV-GFP-Topaz (Angers et al. (2000), supra) in frame with the YFP coding sequence.

The CA subdomain was included in each mutant in order to allow for immunoprecipitation by our anti-CA antibody. 293T cells were co-transfected with the Gag/Rluc and Staufen-HA expressors. In the cell lysates, Gag/Rluc deletion mutants and Staufen-HA were all expressed at comparable levels, except for pr55^{Gag}/Ruc which was consistently expressed at low levels (FIG. 16B). Following immunoprecipitation with anti-CA antibodies, co-precipitated proteins were analyzed by Western blotting using anti-HA to detect Staufen-HA (FIG. 16C). Although each Gag mutant was immunoprecipitated at comparable levels (FIG. 16C, bottom panel), Staufen-HA was only detected in the immunoprecipitates when the NC subdomain was expressed (lanes 1, 4 & 5). These results show that Staufen interacts with pr55^{Gag} with a major contribution from the NC domain. Negligible amounts of Staufen-HA co-precipitated with CA/Rluc and MA-CA/Rluc. Moreover, these results suggest that the interaction does not require the expression of additional viral components.

### EXAMPLE 26

### Staufen and HIV-1 NC Domain of pr55^{GAG} Directly Interact in Live Cells

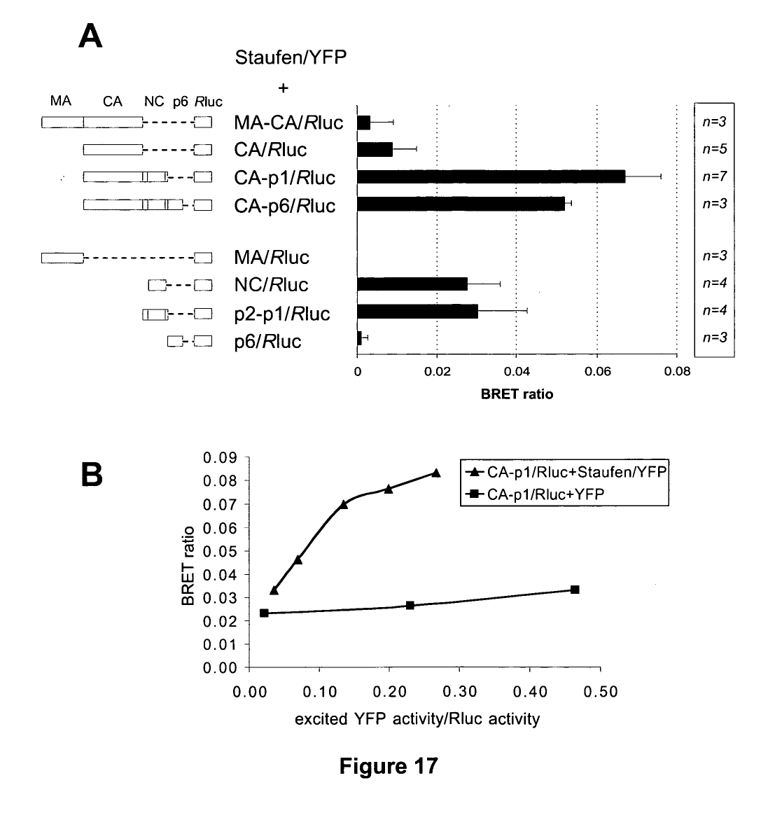

To determine whether the characterized Staufen/pr55^{Gag} interaction above was not an artifact that may result from the preparation of detergent-solubilized cellular extracts, we performed protein interaction assays in live cells using bioluminescence resonance energy transfer (BRET) analysis (Angers et al., 2000, supra). For this method, a BRET is achieved when two proteins directly interact (with a physical separation of approximately <50 Å). Energy from the donor luciferase molecule is transferred to the red-shifted GFP (YFP) molecule acceptor, which then produces a fluorescent light emission to produce a positive BRET signal, calculated as described below. Staufen was fused to YFP (acceptor) and all of the Gag mutants were fused to Rluc (donor) as shown in FIG. 17A.

In detail, 293T cells (2×10⁶) were co-transfected with cDNAs coding for Staufen/YFP and different Gag/Rluc fusion proteins. Forty hours post-transfection, cells were washed in PBS, collected in 1 mL of PBS containing 5 mM EDTA and then diluted to 10⁶ cells/mL. Coelenterazine (Molecular Probes) was added at a final concentration of 5 μM. Luminescence and fluorescence were quantitated with a Fusion α-FP apparatus (Perkin Elmer-Can berra Packard BioScience). Three measures were obtained: first, light emits at 475-480 nm by Rluc; second, emission fluorescence at 525-530 nm without excitation due to energy transfer from Rluc to YFP; third, emission fluorescence at 525-530 nm after excitation at 485 nm to measure total expression of YFP fusion proteins. “BRET ratio” was defined as [(emission at 510-590 nm)-(emission at 440-500 nm)ₓCf]/(emission at 440-500 nm) where Cf corresponds to (emission at 510-590 nm)/(emission at 440-500 nm) for Rluc-fused Gag-mutant expressed alone in the same experiments, as previously described (Angers et al., 2000, supra). To ensure that the BRET ratios reflect a real interaction between fusion proteins, dose-response assays were performed for each tested protein. 293T cells were co-transfected with constant amounts of different Gag/Rluc fusion proteins and increasing concentrations of either Staufen/YFP or YFP alone. At each concentration the BRET ratios were determined as described above and plotted as a function of the ratio of “total YFP activity after excitation/total Rluc activity”. These curves allow us to compare BRET ratios obtained with Staufen/YFP (due to specific interaction with Gag/Rluc) and YFP alone (control) at the same expression levels. In these conditions, whereas BRET ratios linearly increase with YFP concentrations, they more rapidly increase and eventually saturate as the expression level of Staufen/YFP increases, reflecting a specific interaction between Staufen and Gag.

As mentioned above, 293T cells were cotransfected with Staufen-YFP and each of the Gag/Rluc expressors and BRET ratios were determined in live cells 40 h post-transfection. Because full-length pr55 Ruc was poorly expressed (FIG. 17B), it was not included in this set of experiments. As seen in FIG. 17A, positive BRET ratios were always observed when Staufen-YFP was co-expressed with Gag mutants containing the NC domain (CA-p1/Rluc, CA-p6/Rluc, p2-p1/Rluc, NC/Rluc). In contrast, negligible BRET ratios were obtained when the Gag fragments lacked the NC domain (MA-CA/Rluc, CA/Rluc, MA/Rluc, p6/Rluc). Positive BRET ratios were consistently 2- to 3-fold higher when NC was expressed in the form of a precursor (CA-p1 or CA-p6/Rluc) than when it was expressed as a mature protein (NC/Rluc). Dose-response assays with cells transfected with Staufen/YFP or YFP alone provided further evidence that this association in live cells was specific (FIG. 17B and see above). These data confirm our results obtained in the co-immunoprecipitation analyses and further show that Staufen and pr55^{Gag} interact directly in live cells. Here, the pr55^{Gag} interacting domain was mapped to the NC domain and the data also suggest that Staufen has higher affinity for the pr55^{Gag} than for the mature Gag proteins.

### EXAMPLE 27

### HIV-1 Genomic RNA is Found in the Staufen Immune Complex

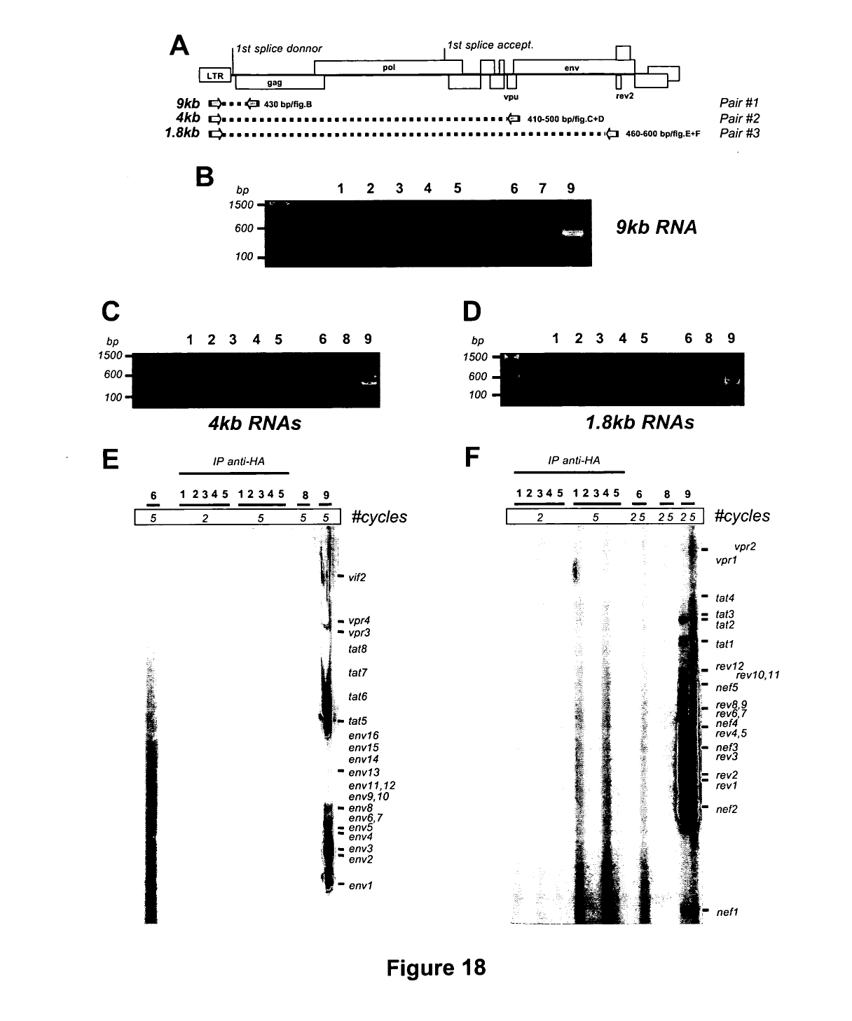

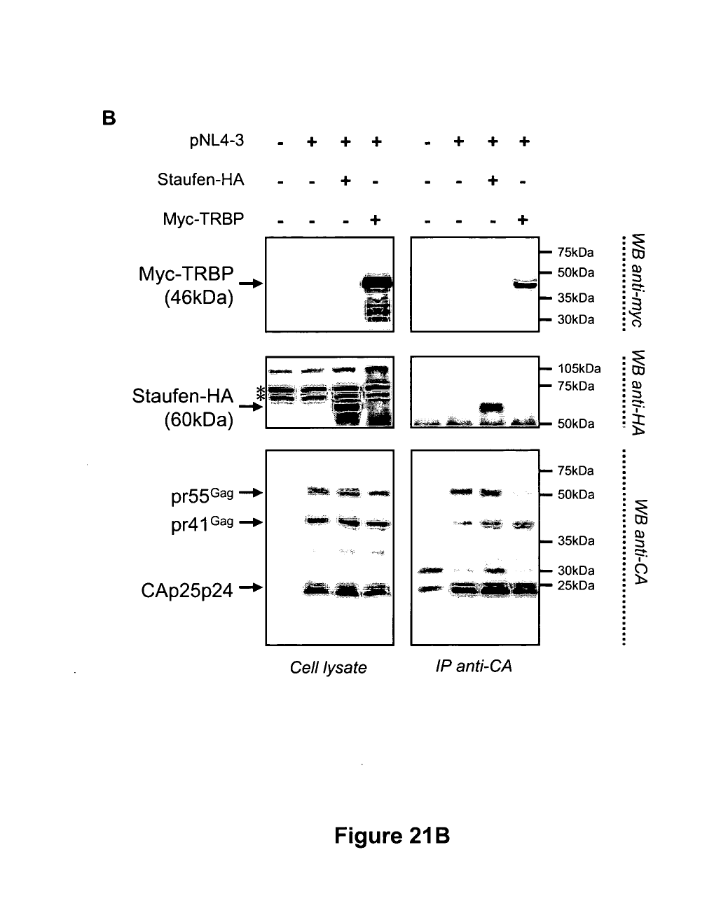

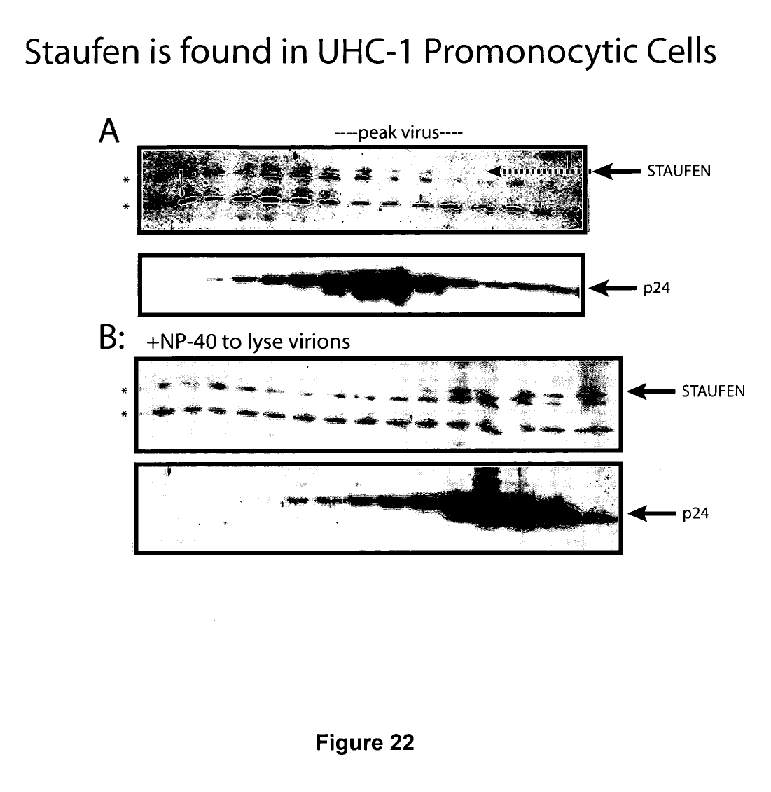

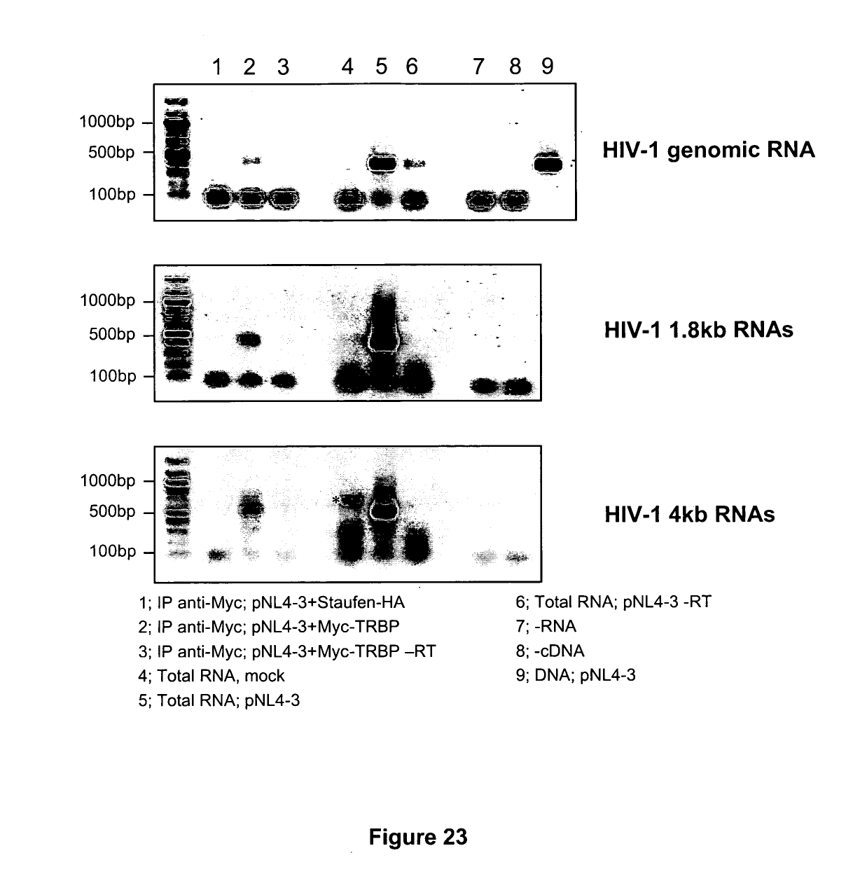

A direct correlation between the level of expression of Staufen and the abundance of genomic RNA encapsidated into virus particles was documented earlier above (Mouland et al., 2000, supra . This suggested that Staufen was implicated in the selection of genomic RNA for encapsidation. While the NC domain of pr55^{Gag} is the principal mediator of this process (Zhang et al. (1997), J. Virol. 71:6765-7649) that depends on an interaction between pr55^{Gag} and genomic RNA (De Guzman et al. (1998), Science 279, 384-388), the relationship between Staufen- and pr55^{Gag}-mediated genomic RNA encapsidation was not clear. Therefore, to further characterize the Staufen-pr55^{Gag} complex and the relationship between Staufen and HIV-1 RNA, we attempted to identify the Staufen-associated HIV-1 RNA species in co-immunoprecipitation assays using our Staufen proviral constructs. 293T cells were transfected with pNL4-3/Staufen-HA and mutant proviruses, and cell extracts were immunoprecipitated using anti-HA. The identification of the co-immunoprecipitated HIV-1 RNA species was determined by RT-PCR with 3 primer pairs that recognized either the genomic RNA, the 4 kb or the 1.8 kb spliced RNAs, as previously described (Purcell, et al. (1993), J. Virol. 67:6365-78) and illustrated in FIG. 18A. Using primer pair #1 for genomic RNA (see below), a specific DNA product of the expected size was detected in the immunoprecipitates isolated from HIV-1/Staufen-HA lysates (FIG. 18B; lane 3). The amplification was specific since there was no signal when the AMV reverse-transcriptase was omitted. In contrast, RT-PCR from HIV-1 alone (FIG. 18B, lane 2), or from the pNL4-3/Staufen^{F135A}-HA or pNL4-3/dsRBD2-4-HA immunoprecipitates did not lead to a detectable PCR product using the primer pair for genomic RNA (FIG. 18B, lanes 4-5). Using the same immunoprecipitated RNA extracts, RT-PCR was repeated with primer pairs specific for spliced HIV-1 RNAs. In both cases, there was no evidence of a PCR product following 35 cycles (FIGS. 18C and 6D). To further rule out the presence of HIV-1 spliced RNAs following the RT reaction, PCR was performed and the last two-or five PCR cycles were performed in the presence of [³²P]dCTP. PCR products were resolved on 6% polyacrylamide gels. As shown in FIG. 18E-F an association with the 4 and 1.8 kb RNA species was not detected even in these conditions. The expected pattern of spliced RNAs was obtained using cell extracts from pNL4-3-expressing cells using this strategy [FIG. 18E-F; (Purcell, et al., 1993, supra)]. These results demonstrate that there is selectivity for genomic RNA by Staufen in HIV-1-producing cells supporting a role for Staufen in the selection of genomic RNA for encapsidation. This immunoprecipitation/RT-PCR analysis was also performed for endogenous Staufen during the expression of HIV-1 and the results also demonstrate that HIV-1 genomic RNA selectively co-immunoprecipitates with Staufen (data not shown). Furthermore, similar analyses were performed with the related dsRNA-binding protein TRBP. Whereas Staufen exhibited selectivity for the 9 kb RNA as described above, TRBP was found to co-immunoprecipitate with all HIV-1 RNA species (FIG. 21).

In detail, to identify Staufen-associated RNAs, Staufen-HA was first immunoprecipitated with a monoclonal anti-HA antibody as previously described (Mouland et al., 2000, supra). The IP-pellets were incubated with 20 μg RNase-free DNase I (Invitrogen) for 1 h at 37° C. and then with 50 μg proteinase K for 30 min at 42° C. Co-precipitated RNAs were extracted as described (Mouland et al., 2000, supra), dissolved in DEPC-treated water and reverse transcribed with random primers and AMV reverse-transcriptase using First Strand cDNA Synthesis Kit (Roche Applied Bioscience). The resulting cDNAs were PCR-amplified using Taq polymerase (Roche Applied Bioscience) and either of three primer pairs that discriminate between the three HIV RNA size classes. All three sets include the same sense primer (5′-CTGAGCCTGG GAGCTCTCTG GC-3′, SEQ ID NO.48) present in the TAR region of all HIV-1 RNA species (Mouland et al., 2000, supra). Primer pair#1 specifically amplify a 430 bp fragment from the genomic HIV-1 RNA (9 kb) since the antisense primer (5′-TCCAGTGATT TTTTTCTCCA TGCTTGCCCA TACTATATGT TT-3′, SEQ ID NO.49) is located in the gag gene. Primer pair #2 preferentially amplify PCR-fragments of 410-500 bp from the spliced 4 kb HIV-1 RNA species since the antisense primer (5′-TCATTGCCAC TGTCTTCTGC TCT-3′, SEQ ID NO.50) hybridizes in the region of the vpu gene. This region is absent in spliced 1.8 kb HIV-1 RNA species. Primer pair #3 preferentially amplify 460-600 bp fragments from the spliced 1.8 kb RNA species, the antisense primer (5′-CCGCAGATCG TCCCAGATAA G-3′, SEQ ID NO. 51) being localized in the second exon of the rev gene. Amplification from genomic and 4 kb spliced RNAs is unlikely in our conditions due to the length of the product. RT-PCR products were analyzed on 0.8% agarose gel. When necessary and to enhance the sensitivity of detection, PCR products were submitted to two or five additional PCR cycles in the presence of 10 μCi of [α-³²P]dCTP as described (Purcell et al., 1993, supra). Labeled PCR products were resolved on 6% denaturing polyacrylamide gels and detected by autoradiography.

### EXAMPLE 28

### Incorporation of an Excess of Staufen into HIV-1 Virions Decreases the Infectivity Thereof

Whatever the particular mechanism of incorporation of hStau into HIV-1, the present invention clearly identifies a new HIV-targeting molecule. It was of an interest to investigate wether the overexpression of Staufen had an effect on infectivity once incorporated into a virion The effects of incorporated hStau on the infectivity of HIV-1 particles were investigated. hStau with pNL4.3 was overexpressed in 293T cells and a corresponding increase in hStau was found in purified virus preparations (FIG. 11A). Equal amounts of virus from pNL4.3- and pNL4.3/hStau-transfected cells were used to infect HeLa-CD4-□Gal (MAGI; Kimpton et al. (1992), J. Virol. 66:2232) and BF-24 (Fortin et al. (1997), J. Virol. 71:3588) indicator cells. Both infectivity assays indicated that an excess amount of hStau in HIV-1 particles has a marked negative effect on virus infectivity [4- and a 6.7-fold decrease in MAGI and BF-24 assays, respectively; FIG. 4B & C]. These data further support the contention that hStau plays an integral role in virus assembly and can contribute to the infectious potential.

### EXAMPLE 29

### Staufen Knock Down By RNA Interference Generates HIV-1 with Compromised Infectivity

Four small interfering (si), double-stranded 21-basepair RNAs to a selected region of Staufen cDNA were chosen and tested for Staufen knock-down efficiency in preliminary experiments. The duplex siRNA 3084 (5′-AAATAGCACAGTTTGGAAACT-3′, SEQ ID NO. 52) was determined to produce the most significant knockdown of Staufen gene expression and was chosen for the experiments presented herein [purchased from Qiagen-Xeragon (Germantown, Md.)]. A control non-silencing (N-S) siRNA (catalog #1022076: 5′-MTTCTCCGMCGTGTCACGT-3′, SEQ ID NO. 53) was purchased from Qiagen-Xeragon and included in either mock or HxBRU (vpr+, vif+, nef−, vpu−)-transfected cells. 293T cells were trypsinized and plated in 6 well plates at 2×10⁵ cells per well for 12-16 h before transfection. Transfections were performed using the Lipofectamine 2000 transfection reagent (Invitrogen, Carlsbad, Calif.) with siRNA at a final concentration of 50 nM. Cells were either transfected with N—S siRNA (mock or HXBRU) or Staufen siRNA 3084. At 24 hr, cells were mock-transfected (KSII with N-S siRNA), or transfected with HxBRU (HxBRU+N-S-RNA), or HxBRU (HxBRU+Staufen siRNA 3084).24 hours later, cells were washed with ice-cold PBS and lysed in NP-40 buffer (100 mM NaCl, 10 mM Tris, 1 mM EDTA, 0.5% NP-40). Virus was pelleted by ultracentrifugation of supernatants. Cytosolic extracts quantitated for protein by the micro-Bradford assay and Staufen and GAPDH were assessed by Western Blot analysis using a mouse monoclonal Staufen antibody (9E9) as described above and a GAPDH antibody (Research Diagnostics, Inc.) Actin was also identified using an anti-actin antibody (not shown). The other half of cellular extract was treated with urea buffer and phenol-chloroform-isoamyl alcohol to extract cytosolic RNA (Mouland et al. (2002), Virolology 292:221-230). RNA was precipitated with ethanol and glycogen carrier (Roche). The RNA pellets were resuspended in DEPC-treated water and quantitated by OD. 1 μg of RNA from each sample was used in RT-PCR analysis using the Superscript One-Step RT-PCR Kit with Platinum Taq (Invitrogen). Either Staufen mRNA (to produce a 337 bp PCR product: Sense: 5′-aatctagaTTTACCAGGGCAGCTCCGAA-3′ (SEQ ID NO. 54); antisense: 5′-aatctagaCMCTCAGACAGCAACTTTMGATGT-3′ (SEQ ID NO.55)) or gapdh RNA exactly as described in reference (Kobinger, G. P. et al. (1997), Gene Ther. 4:868-74) was specifically amplified as described previously (Mouland et al., 2000, supra).

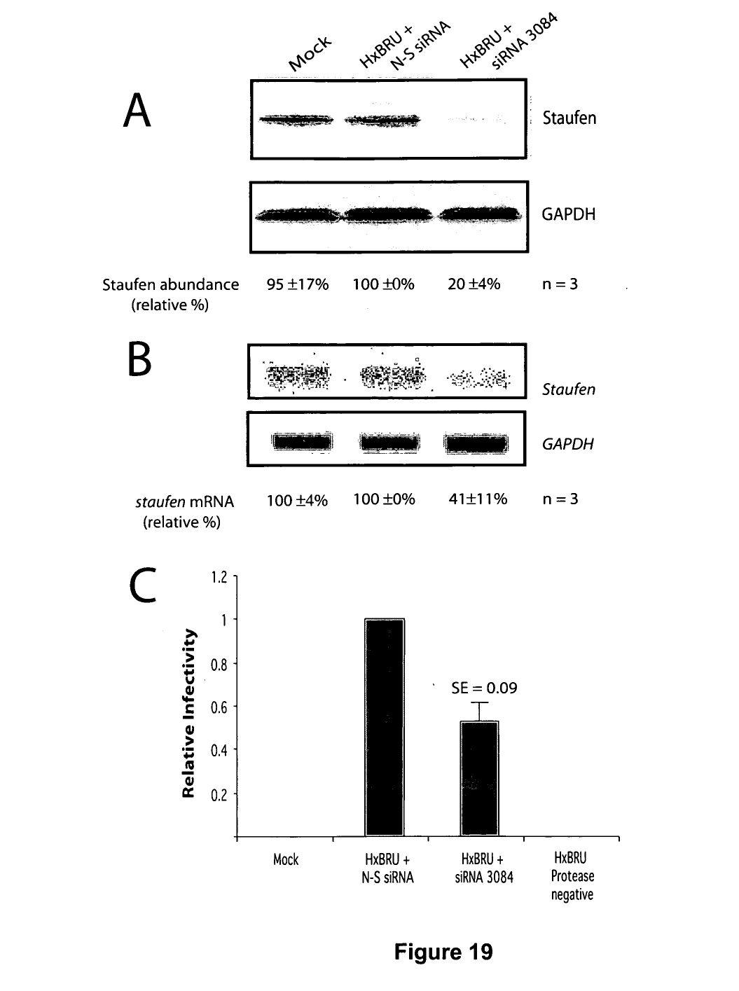

Therefore, Staufen expression was targeted by 21-basepair small interfering RNA duplexes as described above. Cells were either mock transfected (with a non-silencing control RNA duplex) or transfected with pNL4-3 (plus a non-silencing control RNA duplex), or with pNL4-3/Staufen (plus a Staufen siRNA 3084). Staufen siRNA 3084 knocked down Staufen protein expression by 80% (FIG. 19A) and Staufen mRNA levels by almost 60% as shown in RT-PCR analyses (FIG. 19B). Staufen siRNA 3084 did not affect either GAPDH protein and RNA levels (FIG. 19A-B) or those of actin (data not shown), HIV-1 Infectivity Assay

293T cells were treated with a N-S siRNA (Mock or HxBRU alone) or treated with Staufen siRNA 3084 as described above. Virions produced from 293T cells were collected, filtered and pelleted by ultracentrifugation. 8×1 cells CEM-GFP cells (Qervaix, A. et al. (1997), Proc. Natl. Acad. Sci. USA 94:4653-4658) were infected with equivalent amounts (25 ng) of virus as determined by p24 ELISA (Baftisti, P. L. et al. (2003), AIDS Res. & Hum. Retroviruses. 19:767-781, Bounou, S. et al. (2002); J. Virol. 76:1004-14) in the presence of 10 μg/ml of Polybrene (Sigma-Aldrich) and in a final volume of 200 ul of culture medium. 3 h post-infection, CEM-GFP cells were resuspended in 10 ml of fresh medium (RPMI, 10% FBS and 500 ug/ml G418) and incubated for 48 hours. Cells were then pelleted, washed two times with PBS and fixed in 1% formaldehyde in PBS. 105 viable cells were then transferred to a 96-well plate. Fluorecence was measured using an Fusion-Alpha Reader Apparatus (Perkin-Elmer Life Sciences) using an excitation wavelength of 485 nm and an emission wavelength of 520 nm. Data are expressed as relative infectivity where wild-type HIV-1 infectivity (HxBRU treated with a N—S siRNA) was arbitrarily set to a value of 1.

When HIV-1 infectivity was assessed with equal quantities of virus as determined in a p24 ELISA (Battisti, et al., 2003, supra, Bounou et al., 2002, supra), Staufen siRNA 3084 resulted in a two-fold drop in viral infectivity (FIG. 19C). Similar experiments, using another small interfering RNA (siRNA 2), having its sense and antisense sequences on the same strand linked by a small linker sequence (GAAGCTTG of SEQ ID NO:56), were conducted. This particular siRNA folds back on itself to form a short hairpin capable of inducing RNAi (e.g., SEQ ID NO:56). It reduced Staufen protein and mRNA expression with the same efficiency than the siRNA 3084 (data not shown). As Staufen siRNA 3084, siRNA 3085 did not affect either GAPDH protein and RNA levels or those of actin.

As shown above, Staufen overexpression also caused a significant decrease in viral infectivity (Mouland et al., 2000, supra). Taken together with the results of example 28, these data clearly demonstrate and validate that an optimum amount of Staufen expression is required to produce fully infectious viral particles and that Staufen constitute an important therapeutic target in RNA viruses infection in general, and more particularly in HIV-1 infection.

### EXAMPLE 30

### Discussion and Implications of the Role of Staufen in RNA Encapsidation

Because all cells examined until now express hStau, its virion incorporation is indicative of a late role in viral assembly. hStau's ability to bind double-stranded and structured RNAs may result in virion incorporation which would be consistent with a role in the sorting of retroviral genomic RNAs to sites of virus assembly. While the other TAR RNA- and dsRNA-binding proteins have important roles in HIV-1 gene expression and replication, hStau is shown here as the only member that is incorporated into virus particles. Moreover, hStau appears to be incorporated into several retroviruses as well as RNA viruses—but not DNA viruses—suggesting a common role for hStau in the assembly process of RNA viruses.

Overexpression of hStau leads to a marked increase in the amount of hStau in virus preparations (FIG. 11A). As a consequence, virus infectivity is negatively affected (FIG. 11B & C). These results may be explained by steric hindrance or an inappropriate amount of encapsidated viral RNA. Nevertheless, the data herein presented demonstrate that an appropriate amount of incorporated hStau is required to generate infectious viral particles. Accordingly, our infection backcross experiments using MT4 and Jurkat cells show that the quantity of incorporated hStau is independent of the cell line, contrary to what was found for cellular proteins within the HIV-1 envelope. Briefly, backcross experiments were performed using MT4 and Jurkat T cells. 50 ng p24 pNL4.3 virus equivalents were used to infect Jurkat and MT4 cells. Cells were washed extensively and allowed to become productively infected. Virus was then harvested from each culture, purified, and the same amount was used to infect the other cell type. Virus was again harvested and hStau was evaluated in the all virus preparations by Western analysis using equal quantities of virus from each preparation. hStau levels per ng p24 were relatively constant in all virus preparations, in contrast to what was found for proteins embedded in the HIV-1 envelope [cf. Bastiani et al. (1997), J. Virol. 71:3444] Based on the apparent role of Staufen in RNA viruses in general, such an approach is expected to also be beneficial for other RNA viruses.

TAR RNA-binding in HIV-1 has a critical role in transcription (Gatignol et al. (1991), Science 29:1597, Arya et al. (1985), Science 229:69, Dayton et al. (1986), Cell 44:941), but has also been shown to regulate viral gene expression post-transcriptionally (Ferrandon et al. (1997), EMBO J. 16:1751-1758, Park et al. (1994), Proc. Natl. Acad. Sci. U.S.A. 91:4713). All members of the dsRNA-binding protein family are associated with the translational machinery including xlrbpa which can bind to free ribosomal subunits and mRNAs in Xenopus oocytes (Eckman et al. (1997), J. Cell Biol. 138:239-253), and PKR that was recently shown to be associated with 40S ribosomal subunits (Zhu et al. (1997), J. Biol. Chem. 272:14434). Furthermore, TRBP can modulate PKR phosphorylation of elF-2 □ to modulate HIV-1 gene expression (Benkirane et al. (1997), EMBO J. 16:611-624). TRBP was also recently shown to interact with Tax of HTLV-1 (Donzeau et al. (1997), J. Virol. 71:2628) and this could modulate gene expression at transcriptional and/or post-transcriptional levels. Likewise, additional regulatory roles for hStau are expected to be uncovered. In support of this are preliminary studies that indicate that hStau can markedly relieve the TAR-mediated translational repression in vitro in reticulocyte lysates. Highly purified hStau (Wickham et al. (1996), Genomics 36:527) was incubated with a TAR-less RNA or a TAR-containing RNA generated by in vitro transcription of SP6CAT and SP6TARCAT plasmids [Parkin N. T. et al., EMBO J. 7, 2831 (1988)]. TAR dramatically reduced the amount of CAT protein produced in vitro translation as reported previously (ibid.). A dose-dependent derepression of CAT synthesis was observed when the TAR-CAT RNA was preincubated with recombinant hStau. There were no marked effects on CAT protein levels from the TAR-less RNA. This indicates that hStau has several functional parallels to its metazoan counterpart, and furthermore, its role in HIV-1 replication is likely to be multifaceted.

The present disclosure demonstrates that the dsRNA binding protein, Staufen, interacts with the NC domain of HIV-1 pr55^{Gag} during HIV-1 gene expression. This interaction was demonstrated in in vitro assays using both proviral and expression constructs and in live cells using BRET analysis, indicating that it is direct and does not require other viral components. Cellular Staufen/pr55 complexes only contain HIV-1 genomic RNA but not the spliced HIV-1 RNA species. They are resistant to membrane solubilization by detergent but are sensitive to trypsin proteolysis, two characteristics of the cytoplasmic assembly DRC (Lee et al., 1999, supra, Lee et al., 1998, supra). Taken together, these results further support a role for Staufen during virus assembly and genomic RNA encapsidation. Additionally, we showed that Staufen overexpression resulted in decreased virus infectivity but at the same time resulted in enhanced genomic RNA packaging in virions (Mouland et al., 2000, supra). siRNA results shown in FIG. 19 further support a role for Staufen in virus assembly or at another level that impacts significantly on the infectious potential of virions. More recent work supports the notion that Staufen acts during assembly in that Staufen appears to affect virion morphogenesis (not shown). It will be interesting to determine at what stage Staufen is acting in the assembly process since other cellular proteins such as HP68 have been shown to be associated with several Gag assembly intermediates (Zimmerman, C. et al. (2002), Nature 415:88-92).

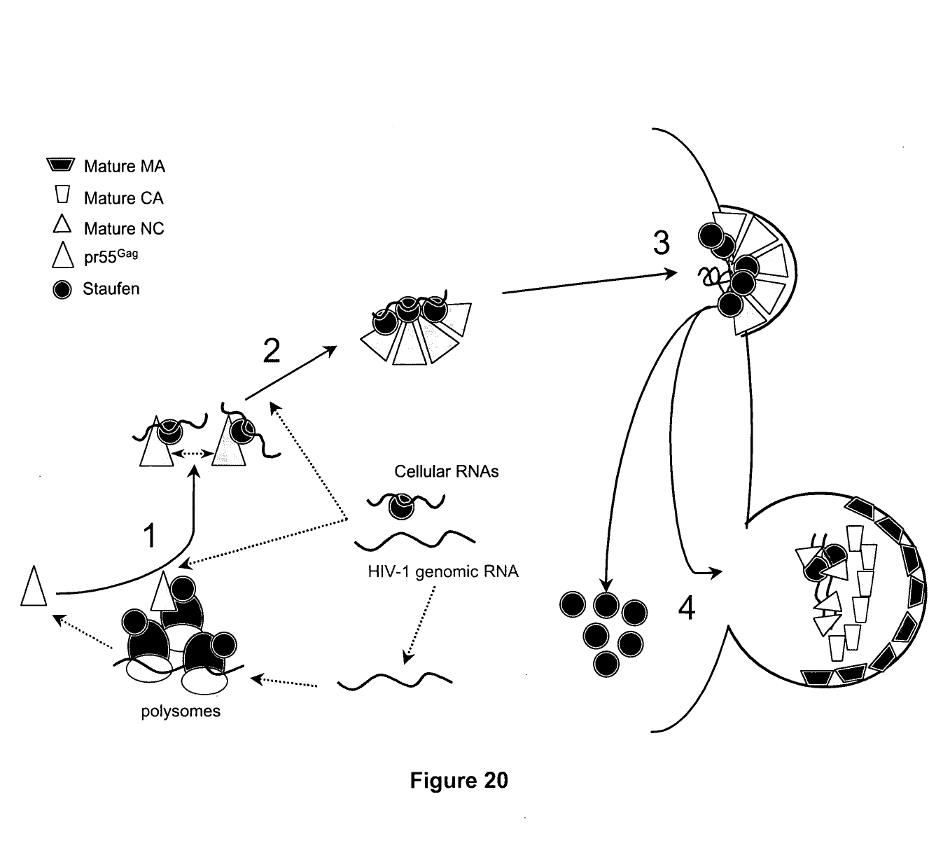

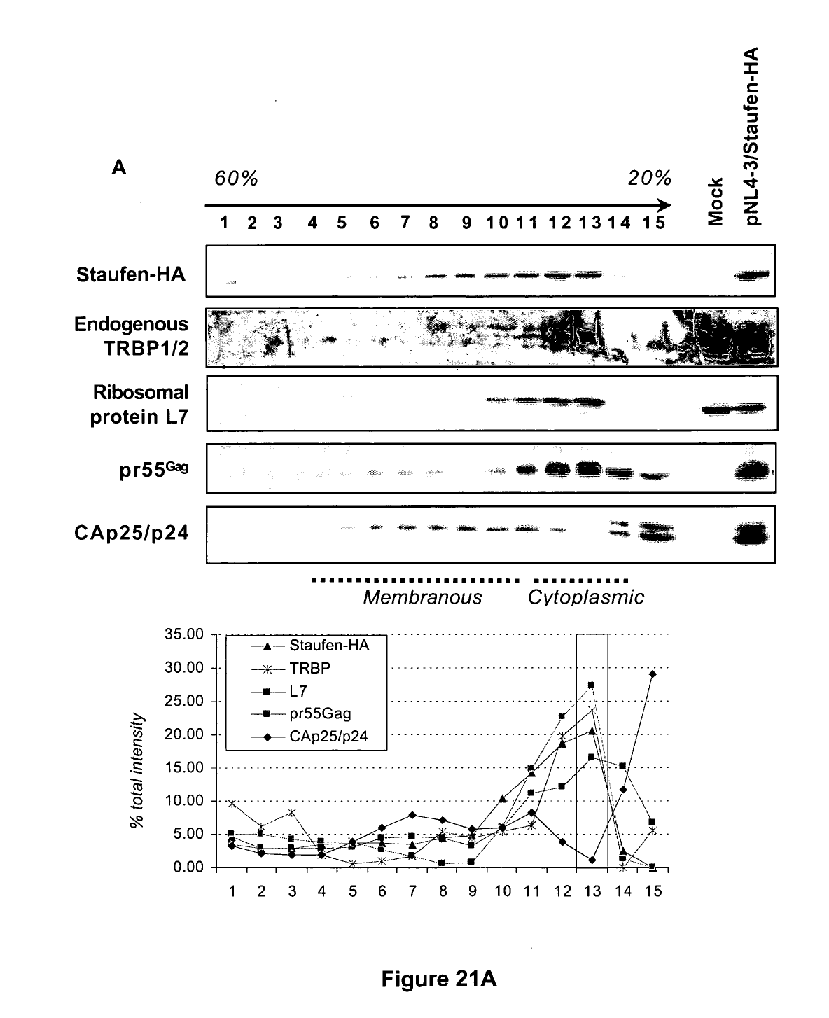

Confocal imaging analysis of cells transfected with pNL4-3/Staufen-HA provirus did not reveal any apparent changes in Staufen's cellular distribution when compared to cells expressing Staufen alone (not shown). This is in contrast to the dramatic nuclear relocalization of Staufen when influenza virus NS1 protein is expressed (Falcon, A. et al. (1999) Nucleic Acids Research 27:2241-7). Partial colocalization was found between HIV-1 Gag and Staufen suggesting that a proportion of Staufen is associated to pr55^{Gag} at any one time (data not shown). This interaction was substantiated by several assays including yeast two hybrid (Mouland et al., 2000, supra), in vitro co-immunoprecipitation and BRET analyses in live cells (FIGS. 15-17). The interaction appeared to be direct and was consistently found to be in the same order of magnitude than that found for Gag-Gag in these assays indicating that this may reflect a transient association that is only required for specific steps in the viral replication cycle and/or to allow a fine-tuned regulation of assembly steps (FIG. 20). Indeed, dynamic and transient formation and modification of ribonucleoprotein complexes is a major hallmark of RNA transport and localization (St Johnston, D. (1995), Cell 81,161-170) and this appears to be also the case in the formation of an HIV-1 assembly complex (Tritel, M. et al. (2000), J. Virol. 74:5845-55, Zimmerman et al., 2002, supra).

Immunoelectron microscopy experiments revealed cytoplasmic assembly intermediates representing small Gag oligomers of about 10 molecules (Nermut, M. V. et al. (2003), Virology. 305:219-27), likely corresponding to the previously described cytoplasmic DRC (Lee et al., 1999, supra, Lee et al., 1998, supra) and the complexes identified immediately following pr55^{Gag} synthesis (Tritel et al., 2000, supra). Our results favor the idea that Staufen is recruited early by pr55^{Gag} in this cytoplasmic assembly DRC to participate in the first events of NC-mediated Gag multimerization. Consistently, Staufen complexes identified in ourfractionation studies have all the characteristics of the cytoplasmic DRC (FIG. 14). Furthermore, the Staufen/pr55^{Gag}, interaction does not require translocation to the membrane since the interaction of Staufen with Gag occurs when the MA domain is absent (FIGS. 16-17). Since Staufen likely functions in mRNA trafficking in the cytoplasm (Kohrmann, M. et al. (1999), Mol Biol Cell. 10:2945-53), it could participate in the translocation of the pr55^{Gag} assembly complexes and/or play a role in the recruitment of genomic RNA to sites of viral assembly.

### EXAMPLE 31

### Proposed Model for Staufen Involvement in the Post-Transcriptional Steps of HIV-1 Lifecyle

The results disclosed herein point to a role of Staufen in the post-transcriptional steps of the HIV-1 lifecycle (FIG. 20). Staufen is likely to be recruited early during viral assembly by newly synthesized pr55^{Gag} via the NC domain (FIG. 20, Step 1). Staufen's association with ribosomes (Luo, M. et al. (2002), Biochem J. 365:817-24) and the cosedimentation with ribosomes in sucrose gradients (FIG. 14) indicate that a Staufen-pr55 Ga-ribosome complex could represent an early assembly complex intermediate in the HIV-1 replication cycle. Cellular mRNAs and/or HIV-1 genomic RNA may then compete with ribosomes to release Staufen-Gag complexes (FIG. 20, Step 1). Consistently, a molecular competition is proposed to exist between ribosomal protein L18 and dsRNA for the dsRNA-binding protein, PKR (Kumar, K. U. et al. (1999), Mol. & Cell. Biol. 19:1116-25), and another that was proposed to exist between ribosomes and genomic RNAMLV for MoMLV Gag that may result in the encapsidation of ribosomes instead of genomic RNAMLV (Muriaux, D. et al. (2002), J. of Virology. 76:11405-1335). Staufen could also contribute to the oligomerization of pr55^{Gag} by recruiting RNA, a component that was shown to be a necessary cofactor for this (Burniston, M. T. et al. (1999), J. Virol. 73:8527-408, Cimarelli et al., 2000, supra), or likewise promote the association of genomic RNA with pr55^{Gag} (FIG. 20, Step 2). Because Staufen is a cytoskeleton-associated protein and is involved in the transport of ribonucleoprotein complexes, Staufen may mediate the transport of this assembly complex intermediate toward the plasma membrane, where a ternary assembly complex of Staufen, pr55^{Gag} and genomic RNA is maintained to lead to genomic RNA encapsidation (FIG. 20, Step 3). While RNA does not appear to be an important cofactor for the g association (FIG. 15D), genomic RNA is necessary for Staufen incorporation since NC and packaging signal proviral mutants generate virus particles that are devoid of Staufen (Mouland et al., 2000, supra). Because a single point mutation in the Staufen dsRBb3 abrogates both the genomic RNA and pr55^{Gag} association, a dual role for this domain is suggested, as demonstrated for several other related, dsRNA binding proteins such as PKR and TAR-RNA binding protein (Daher, A. et al. (2001), J. Biol. Chem. 276:33899-905). Knocking down Staufen expression by siRNA is likely to disrupt early or subsequent steps of assembly through Staufen's interactions with pr55^{Gag} and RNA and contribute to the generation of poorly infectious virus particles (FIG. 19).

Sucrose gradient, co-immunoprecipitation and BRET analyses strongly support a preferential association of Staufen to pr55^{Gag}, and not to mature Gag proteins (FIGS. 14-17). This dictates that during Gag maturation by the viral protease, Staufen is likely to be progressively excluded from Gag assembly complexes by modulation of its affinity for Gag (FIG. 20, Step 4). Structural changes in Gag during maturation were previously proposed to explain the partial exclusion of cyclophilin A from HIV-1 (Bristow, R. et al. (1999), J. Acquir. Immune Defic. Syndr. Hum. Retrovirol. 20:334-6, Tang, C. et al. (2002), Nat Struct Biol. 9:537-43).

Staufen expression levels are important for HIV-1 replication. For example, Staufen overexpression enhances not only the Staufen copy number within virus particles, but also correspondingly enhances the number of HIV-1 genomic RNA copies packaged into the virion (Mouland et al., 2000, supra). It also results in morphological changes of virus particles (not shown) and this is likely to be due to Staufen that is over-represented in the Staufen-pr55^{Gag}-genomic RNA ternary complex during assembly. Equally so, Staufen knockdown (FIG. 19) could also disrupt the composition of this ternary complex at this step of the assembly process. Genomic RNA dimerization during HIV-1 assembly may play a role in this switch of Staufen to associate with genomic RNA, especially since Drosophila Staufen's association with bicoid mRNA requires RNA dimerization (Ferrandon et al., 1997, supra). Staufen's selective association to a complex in which HIV-1 genomic RNA is found (FIG. 18), especially when compared to the absence of specificity exhibited by TRBP, is indicative a role of Staufen in the fate of this RNA species during HIV-1 expression. If Staufen participates in the selection of genomic RNA by NC for encapsidation in the context of this Staufen-pr55^{Gag}-genomic RNA complex for instance, we can anticipate that the fine-tuned regulation of the amounts of Staufen associated with fully assembled viral particles could represent a mechanism by which the appropriate number of genomic RNA molecules is packaged into HIV-1 to generate fully infectious viral particles. The experiments presented here and previously (Mouland et al., 2000, supra) clearly support this model.

In light of the negative impact of Stau overexpression and Stau knock down on viral infectivity, Stau is clearly a suitable target for an anti-HIV-1 strategy. Furthermore, in light of the demonstration that hStau is incorporated into other retroviruses as well as Reovirus, Staufen also constitute a suitable target for anti RNA-virus therapy in general. A number of methods directed at significantly modulate the level of Staufen can now be used to decrease the infectivity of RNA viruses.

Although the present invention has been described hereinabove by way of illustrative embodiments thereof, it will be appreciated by one skilled in the art from reading of this disclosure that various changes in form and detail can be made without departing from the spirit and nature of the invention as defined in the appended claims.

### REFERENCES

1. Ainger, K. et al. (1993), Transport and localization of exogenous myelin basic protein mRNA microinjected into oligodendrocytes, J. Cell. Biol. 123:431-441;

2. Aloyz, R. S. et al. (1995), Processing of the L5-67 precursor peptide and characterization of LUQIN in the central nervous system of Aplysia californica, Peptides 16: 331-338;

3. Angers, S. et al. (2000), Detection of beta 2-adrenergic receptordimerization in living cells using bioluminescence resonance energy transfer (BRET), Proc. Natl. Acad. Sci. USA 97:3684-9;

4. Arya, S. K. et al. (1985), Science 229:69; 5. Bachand, F. et al. (1999), Incorporation of Vpr into human immunodeficiency virus type 1 requires a direct interaction with the p6 domain of the p55 gag precursor, J. Biol. Chem. 274:9083-91;

6. Barr, P. J. et al. (1987), U.C.L.A. Symp. Mol. Cell. Biol. New Ser. 43:205;

7. Bass, 2001, Nature, 411:428-429;

8. Bassell, G. et al. (1997), mRNA and cytoskeletal filaments, Curr. Opin. Cell Biol. 9:109-115;

9. Bastiani et al. (1997), J.Virol. 71:3444;

10. Battisti, P. L. et al. (2003), Additive activity between the TRBP and Cyclin T1 on HIV-1 expression and viral production in murine cells, AIDS Research and Human Retroviruses 19:767-781;

11. Benkirane, M. et al. (1997), Oncogenic potential of TAR RNA binding protein TRBP and its regulatory interaction with RNA-dependent protein kinase PKR, EMBO J. 16:611-24;

12. Bergeron, D. et al. (1991), J. Virol. 65:7;

13. Berkowitz, R. D. et al. (1995), Virology 212:718;

14. Bounou, S. et al. (2002), Presence of host ICAM-1 in laboratory and clinical strains of human immunodeficiency virus type 1 increases virus infectivity and CD4(+)-T-cell depletion in human lymphoid tissue, a major site of replication in vivo, J. Virol. 76:1004-14; S. et al. (2002), J. Virol. 76:1004-14

15. Breitwieser, W. et al. (1996), Oskar protein interaction with Vasa represents an essential step in polar granule assembly, Genes & Dev. 10: 2179-2188;

16. Bristow, R. et al. (1999), Human cyclophilin has a significantly higheraffinity for HIV-1 recombinant p55 than p24, J. Acquir. Immune Defic. Syndr. Hum. Retrovirol. 20:334-6;

17. Broadus, J. et al. (1998), Staufen-dependent localization of prospero mRNA contribules to neuroblast daughter-cell fate, Nature 391:792-795;

18. Burniston, M. T. et al. (1999), Human immunodeficiency virus type 1 Gag polyprotein multimerization requires the nucleocapsid domain and RNA and is promoted by the capsid-dimer interface and the basic region of matrix protein, J. Virol. 73:8527-40;

19. Cimarelli, A. et al. (1999), Translation elongation factor 1-alpha interacts specifically with the human immunodeficiency virus type 1 Gag polyprotein, J. Virol. 73:5388-401;

20. Cimarelli, A. et al.(2000), Basic residues in human immunodeficiency virus type 1 nucleocapsid promote virion assembly via interaction with RNA, J. Virol. 74:3046-57;

21. Cogniaux, J. et al. (1990), J. Immunol. Meth. 128:165;

22. Cosentino, G. P. et al. (1995),. Double-stranded-RNA-dependentprotein kinase and TAR RNA-binding protein form homo- and heterodimers in vivo, PNAS 92:9445-9449;

23. Crino, P. B. et al. (1996), Molecular characterization of the dendritic growth cone: regulated mRNA transport and local protein synthesis, Neuron 17:1173-1187;

24. Daher, A. et al. (2001), Two dimerization domains in the trans-activation response RNA-binding protein (TRBP) individually reverse the protein kinase R inhibition of HIV-1 long terminal repeat expression, J. Biol. Chem. 276:33899-905;

25. Dannull, J. et al. (1994), EMBO J. 13:1525;

26. Davis, L. et al. (1987), Selective dendritic transport of RNA in hippocampal neurons in culture, Nature 330:477-479;

27. Dayton et al. (1986), Cell 44:941;

28. De Guzman, R. N. et al. (1998), Structure of the HIV-1 nucleocapsid protein bound to the SL3 psi-RNA recognition element, Science 279:384-8;

29. DesGroseillers, L. et al. (1996), Localization of a human double-stranded RNA-binding protein gene (STAU) to band 20q13.1 by fluorescence in situ hybridization, Genomics 36:527-529;

30. Deshler, J. O. et al. (1997), Localization of Xenopus Vg1 mRNA by Vera protein and the endoplasmic reticulum, Science 276:1128-1131;

31. Donzeau, M. et al. (1997), J. Virol. 71:2628;

32. Eckmann, C. R. et al. (1997), Xlrbpa, a double-stranded RNA-binding protein associated with ribosomes and heterogeneous nuclear RNPs, J. Cell Biol. 138:239-253;

33. Elbashir et al., 2001, Genes Dev. 15:188;

34. Elbashir et al., 2001, Nature, 411:494-498;

35. Elisha, Z. et al. (1995), Vg1 RNA binding protein mediates the association of Vg1 RNA with microtubules in Xenopus oocytes, EMBO J. 14:5109-5114;

36. Ephrussi, A. et al. (1991), Oskar organizes the germ plasm and directs localization of the posterior determinant nanos, Cell 66:37-50;

37. Erdelyi, M. et al. (1995), Requirement for Drosophila cytoplasmic tropomyosin in oskar mRNA localization, Nature 377:524-527;

38. Falcon, A. M. et al. (1999), Interaction of influenza virus NS1 protein and the human homologue of Staufen in vivo and in vitro, Nucl. Acids Res. 27:2241-7;

39. Ferrandon, D. et al. (1994), Staufen protein associates with the 3′UTR of bicoid mRNA to form particles that move in a microtubule-dependent manner, Cell 79:1221-1232;

40. Ferrandon, D. et al. (1997), RNA-RNA interaction is required for the formation of specific bicoid mRNA 3′ UTR-Staufen ribonucleoprotein particles, EMBO J. 16:1751-1758;

41. Forristall, C. et al. (1995), Patterns of localization and cytoskeletal association of two vegetally localized RNAs, Vg 1 and Xcat-2, Development 121:201-208;

42. Fortin, J. F. et al. (1997), J. Virol. 71:3588;

43. Fortin, J. F. et al. (1998), J. Virol. 72:2105;

44. Gatignol, A. et al. (1991), Science 29:1597;

45. Gatignol, A. et al. (1993), Relatedness of an RNA-binding motif in human immunodeficiency virus type 1 TAR RNA-binding protein TRBP to human P1/dsl kinase and Drosophila Staufen,. Mol. Cell. Biol. 13:2193-2202;

46. Gauthier at al. (1987), Nucl. Acids, Res. 15:6625-6641;

47. Gazzaley, A. H. et al. (1997), Differential subcellular regulation of NMDAR1 protein and mRNA in dendrites of dendate gyrus granule cells after perforant path transection, J. Neurosci. 17:2006-2017;

48. Gervaix, A. et al. (1997), A new reporter cell line to monitor HIV infection and drug susceptibility in vitro, Proc. Natl. Acad. Sci. USA, 94:4653-4658;

49. Gorelick, R. et al. (1990), J. Virol. 64:3207; 50. Gorelick, R. et al. (1993), J. Virol., 67:4027;

51. Hochstenback, F. et al. (1992), Endoplasmic reticulum resident protein of 90 kilodaltons associates with the T- and B-cell antigen receptors and major histocompatibility complex antigens during assembly, PNAS 89:4734-4738;

52. Huang, Y. et al. (1997), J. Virol. 71:4378;

53. Inoue et al. (1987), FEBS Lett. 215:327-330;

54. Inoue et al. (1987), Nucl. Acids. Res. 15:6131-6148;

55. Jockers, R. et al. (1996), Newmolecularandstructural determinants involvedin b2-adrenergic receptor desensitization and sequestration, J. Biol. Chem. 271:9355-9362;

56. Kang, H. et al. (1996), A requirement forlocalprotein synthesis in neurotrophin-induced hippocampal synaptic plasticity, Science 273:1402-1406;

57. Khorchid, A. et al. (2002), Role of RNA in facilitating Gag/Gag-Pol interaction, J. Virol. 76:4131-7;

58. Kiebler et al.

59. Kilby, M. et al. (2003), New England J. of Medicine, 348(22):2228-2238;

60. Kim-Ha, J. et al. (1991), Oskar mRNA is localized to the posterior pole of the Drosophila oocyte, Cell 66:23-35;

61. Kim-Ha, J. et al. (1995), Translational regulation of oskar mRNA by Bruno, an ovarian RNA-binding protein, is essential, Cell 81:403-412;

62. Kimpton et al. (1992), J. Virol. 66:2232;

63. Kislauskis, E. H. et al. (1997), b-actin messenger RNA localization and protien synthesis augment cell motility, J. Cell Biol. 136:1263-1270;

64. Kloc, M. et al., (1994), Delocalization of Vg 1 mRNA™ from the vegetal cortex in Xenopus oocytes after destruction of Xlsirt RNA, Science 265:1101-1103;

65. Knowles, R. B. et.al. (1996), Translocation of RNA granules in living neurons, J. Neurosci. 16:7812-7820;

66. Kobinger, G. P. et al. (1997), Enhancement of retroviral production from packaging cell lines expressing the human immunodeficiency type 1 VPU gene, Gene Ther. 4:868-74;

67. Kohrmann, M. et al. (1999), Microtubule-dependent recruitment of Staufen-green fluorescentprotein into large RNA-containing granules and subsequent dendritic transport in living hippocampal neurons, Mol Biol Cell. 10:2945-53;

68. Kraut, R. et al. (1996), Inscutable, a neural precursor gene of Drosophila encodes a candidate for a cytoskeletal adaptor protein, Dev. Biol. 174:66-81;

69. Krovat, B. C. et al. (1996), Comparative mutational analysis of the double-stranded RNA binding domains of Xenopus laevis RNA-binding protein A, J. Biol. Chem. 271:28112-28119;

70. Kumar, K. U. et al. (1999), Double-stranded RNAactivated protein kinase (PKR) is negatively regulated by 60S ribosomal subunit protein L18, Mol. & Cell. Biol. 19:1116-25;

71. Kwoh et al. (1989), Proc. Natl. Acad. Sci. USA 86:1173-1177;

72. Kwoh et al. (1990), Am. Biotechnol. Lab. 8:14-25;

73. Lee, Y. M. et al. (1998), Identification and characterization of virus assembly intermediate complexes in HIV-1-infected CD4⁺ T cells, Virology 243:78-93;

74. Lee, Y. M. et al. (1999), Formation of virus assembly intermediate complexes in the cytoplasm by wild-type and assembly-defective mutant human immunodeficiency virus type 1 and their association with membranes, J. Virol. 73:5654-62;

75. Lever et al. (1989), J. Virol. 63:4085;

76. Li, P. et al. (1997), Inscutable and Staufen mediate asymmetric localization and segregation of prospero RNA during Drosophila neuroblast cell divisions, Cell 90:437-447;

77. Lizardi et al. (1988), BioTechnology 6:1197-1202;

78. Long, R. M. et al. (1997), Mating type switching in yeast controlled by asymetric localization of ASH1 mRNA, Science 177:383-387;

79. Luo, M. et al. (2002), Molecular mapping of the determinants involved in human Staufen-ribosome association, Biochem J. 365:817-824;

80. Malek et al. (1994), Methods Mol. Biol., 28:253-260; 81. Martin, K. C. et al. (1997), Synapse-specific, long-term facilitation of Aplysia sensory to motor synapses: a function for local protein synthesis in memory storage, Cell 91:927-938;

82. McBride, M. S. et al. (1997), J. Virol. 71:4544; 83. McCormack, S. J. et al. (1994), Mechanism of interferon action: motif 1 of the interferon-induced, RNA-dependent protein kinase (PKR) is sufficient to mediate RNA-binding activity, Virology 198:92-99;

84. Miele, G. et al. (1996), J. Virol. 70:944;

85. Miller (1988), Ann. Reports Med. Chem. 23:295;

86. Mizuno, A. et al. (1996), AIDS Res. Hum. Retrov. 12:793;

87. Moran et al. (1987), Nucl. Acids Res., 14:5019;

88. Mouland, A. J. et al. (1992), Mol. Endocrinol. 6:1781;

89. Mouland, A. J. et al. (2000), The double-stranded RNA-binding protein Staufen is incorporated in human immunodeficiency virus type 1: evidence for a role in genomic RNA encapsidation, J. Virol. 74:5441-51;

90. Mouland, A. J. et al. (2002), Hypophosphorylation of Poly(A) Polymerase and Increased Polyadenylation Activity are Associated with Human Immunodeficiency Virus Type 1 Vpr Expression, Virology 292:221-230;

91. Muriaux, D. et al. (2002), Murine leukemia virus nucleocapsid mutant particles lacking viral RNA encapsidate ribosomes, J.Virology 76:11405-13;

92. Nakielny, S. et al. (1997), RNA transport, Ann. Rev. Neurosci. 20:269-301;

93. Nermut, M. V. et al. (2003), Time course of Gag protein assembly in HIV-1-infected cells: a study by immunoelectron microscopy, Virology 305:219-27;

94. Okita, T. W. et al. (1994), Targeting of mRNAs to domains of the endoplasmic reticulum, TIBS 4:91-96;

95. Ott et al. (1995), AIDS Res. Hum. Retroviruses 11:1003;

96. Ott et al. (1996) J. Virol. 70:7734;

97. Pachter, J. S. (1992), Association of mRNA with the cytoskeletal framework: its role in the regulation of gene expression, Crit. Rev. Euk. Gene Exp. 2:1-18;

98. Palker et al. (1987) Proc. Natl. Acad. Sci. U.S.A. 84:2479;

99. Palker et al. (1988) Proc. Natl. Acad. Sci. U.S.A. 85:1932;

100. Palker et al. (1989) J. Immunol. 142:3612;

101. Park et al. (1994), Proc. Natl. Acad. Sci. U.S.A. 91:4713;

102. Pokrywka, N. J. et al. (1995), Microtubules are a general component of mRNA localization systems in Drosophila oocytes, Dev. Biol. 167:363-370;

103. Poon et al. (1998), J. Virol. 72:1983;

104. Purcell, D. F. et al. (1993), Alternative splicing of human immunodeficiency virus type 1 mRNA modulates viral protein expression, replication, and infectivity, J. Virol. 67:6365-78;

105. Rings, E. H. H. M. et al. (1994), Protein sorting versus messenger RNA sorting? Eur. J. Cell Biol. 63:161-171;

106. Ross, A. F. et al. (1997), Characterization of a b-actin mRNA zipcode-binding protein, Mol. Cell. Biol. 17:2158-2165;

107. Saudek et al. (1989), N. Engl. J. Med. 321:574;

108. Schmedt, C. et al. (1995), Functional characterization of the RNA-binding domain and motif of the double-stranded RNA-dependent protein kinase DAI (PKR), J. Mol. Biol. 249:29-44;

109. Schumacher, J. M. et al. (1995), Spnr, a murine RNA-binding protein that is localized to cytoplasmic microtubules, J. Cell Biol. 129:1023-1032;

110. Schwartz, S. P. et al. (1992), A 69-kDa RNA-binding protein from Xenopus oocytes recognizes a common motif in two vegetally localized maternal mRNAs, PNAS 89:11895-11899;

111. Song, E. et al. (2003), J. of Virology, 77(13):7174-7181;

112. St. Johnston, D. et al. (1989) Multiple steps in th elocalization of bicoid RNA to the anterior pole of the Drosophila oocyte, Dev. (Suppl.) 107:13-19;

113. St. Johnston, D. et al. (1991), Staufen, a gene required to localize maternal RNAs in the Drosophila egg, Cell 66:51-63;

114. St. Johnston, D. et al. (1992), A conserved double-stranded RNA-binding domain, PNAS 89:10979-10983;

115. St. Johnston, D. (1995), The intracellular localization of messenger RNAs, Cell 81:161-170;

116. Steimer et al. (1986), Virology 150:283;

117. Steward, O. (1997), mRNA localization in neurons: a multipurpose mechanism? Neuron 18:9-12;

118. Takizawa, P. A. et al. (1997), Actin-dependent localization of an RNA encoding a cell-fate determinant in yeast, Nature 389:90-93;

119. Tang, C. et al. (2002), Structure of the N-terminal 283-residue fragment of the immature HIV-1 Gag polyprotein, Nat. Struct. Biol. 9:537-43;

120. Terasaki, M. et al. (1986), Microtubules and the endoplasmic reticulum are highly interdependent structures, J. Cell Biol. 103:1557-1568;

121. Tetzlaff, M. T. et al. (1996), Lack of Drosophila cytoskeletal tropomyosin affects head morphogenesis and the accumulation of oskar mRNA required for germ cell formation, EMBO J. 15:1247-1254;

122. Tiedge, H. et al. (1991), Dendritic localization of neural BC1 RNA, PNAS 88:2093-2097;.

123. Tiedge, H. et al. (1993), Transport of BC1 RNA in hypothalamo-neurohypophysealaxons, J. Neurosci. 13:4214-4219;

124. Tongiorgi, E. et al. (1997), Activity-dependent dendritic targeting of BDNF and TrkB mRNAs in hippocampal neurons, J. Neurosci. 17:9492-9505;

125. Tritel, M. et al. (2000), Kinetic analysis:of human immunodeficiency virus type 1 assembly reveals the presence of sequential intermediates, J. Virol. 74:5845-55;

126. Wagner, R. (1994), Nature, 372:333-335;

127. Walker et al. (1992), Nucleic Acids Res. 20:1691-1696;

128. Walker et al. (1992), Proc. Natl. Acad. Sci. USA 89:392-396;

129. Weiss, 1991, Science 254:1292;

130. Wickham, L. et al. (1991), A bradykinin-like neuropeptide precursor gene is expressed in neuron L5 of Aplysia californica, DNA and Cell Biology 10:249-258;

131. Wickham et al. (1996), Genomics 36:527;

132. Wickham, L. et al. (1999), Mammalian Staufen is a double-stranded-RNA- and tubulin-binding protein which localizes to the rough endoplasmic reticulum, Mol Cell Biol. 19:2220-2230;

133. Wilhelm, J. E. et al. (1993), RNA on the move: The mRNA localization pathway., J. Cell Biol. 123:269-274;

134. Wilsch-Brauninger, M. et al. (1997), A sponge-like structure involved in the association and transport of maternal products during Drosophila oogenesis, J. Cell Biol. 139:817-829;

135. Yao, X. J. et al. (1998), Vpr stimulates viral expression and induces cell killing in human immunodeficiency virus type 1-infected dividing Jurkat Tcells, J. Virol. 72:4686-93;

136. Zauner, W. et al. (1992), Identification of two distinct microtubule binding domains on recombinant rat MAPLB, Eur. J. Cell Biol. 57:66-74;

137. Zhu et al. (1997), J. Biol. Chem. 272:14434;

138. Zimmerman, C. et al. (2002), Identification of a host protein essential for assembly of immature HIV-1 capsids, Nature 415:88-92;

What is claimed is:

1. A short interfering RNA (siRNA) molecule that down regulates the expression of Staufen gene by RNA interference comprising a sense region and an antisense region, wherein said antisense region comprises a sequence complementary to a Staufen RNA sequence and the sense region comprises a sequence complementary to the antisense of said Staufen RNA sequence, and wherein the sense region of said siRNA is at least 95% identical to a portion of Staufen nucleic acid selected from the group consisting of: SEQ ID NO:1, SEQ ID NO:3, SEQ ID NO:5, SEQ ID NO:6, SEQ ID NO:7.

2. The siRNA of claim 1, wherein said siRNA molecule is assembled from two'nucleic acid fragments, wherein one fragment comprises the sense region and the second fragment comprises the antisense region of said siRNA molecule.

3. The siRNA of claim 2, wherein said sense region and said antisense region are covalently connected via a linker molecule.

4. The siRNA of claim 3, wherein said linker molecule is a polynucleotide linker molecule.

5. The siRNA molecule of claims 1, wherein said sense region comprises a 3′-terminal overhang of 1 to 5 nucleotides in length and said antisense region comprises a 3′-terminal overhang of 1 to 5 nucleotides in length.

6. The siRNA molecule of claims 1, wherein said said sense and antisense regions comprise at least one nucleotide that is chemically modified in at least one of sugar, base, or backbone moiety.

7. The siRNA molecule of claims 1, comprising a double stranded region of about 10 to 28 nucleotides in length.

8. The siRNA molecule of claim 7, wherein said siRNA molecule is linked to at least one receptor binding ligand.

9. The siRNA molecule of claim 8, wherein said receptor binding ligand is attached to the 5′-end, the 3′end or both ends of the sense or antisense region of the siRNA molecule.

10. The siRNA molecule of claim 8, wherein said receptor binding ligand is a HIV-1 surface antigen.

11. The siRNA molecule of claim 1, wherein said siRNA molecule is between about 10 to 30 nucleotides in length.

12. The siRNA molecule of claim 1, wherein said Staufen RNA sequence is a portion of a sequence selected from the group consisting of: SEQ ID NO:5, SEQ ID NO:1, SEQ ID NO:7, SEQ ID NO:3, and SEQ ID NO:6.

13. The siRNA molecule of claim 1, wherein said sense region comprises the sequence of SEQ ID NO:52.

14. An expression vector comprising a nucleic acid sequence encoding at least one siRNA molecule of claim 1, in a manner that allows the expression of thereof.

15. The expression vector of claim 14, wherein said siRNA molecule comprises a single strand having complementary sense and antisense region.

16. The expression vector of claim 14, wherein said siRNA molecule comprises two distinct strands having a complementary region.

17. A mammalian cell harboring an expression vector of claim 14.

18. A method for treating or preventing a RNA virus infection in a mammalian subject comprising administering thereto a pharmaceutical composition comrpising an siRNA, that reduces Staufen protein expression by RNA interference thereby lowering virus replication or infectivity.

19. The method of claim 8, wherein the mammalian subject is a human.

20. The method of claim 18, wherein the RNA virus is selected from the group consisting of: HIV-1, HIV-2, CasBr, MLV, and Reovirus.

21. A pharmaceutical composition comprising an siRNA of claim 1 together with a suitable pharmaceutical carrier.

22. A method of inducing targeted RNA interference in a subject, comprising administering thereto an effective amount of the siRNA of claims 1.

23. A pharmaceutical composition for treating or preventing an RNA virus infection in a subject comprising an agent that modulates endogenous Staufen protein expression, together with a suitable pharmaceutical carrier, wherein said agent selected from the group consisting of:
a) a nucleic acid encoding a Staufen protein of SEQ ID NO:2, SEQ ID NO:4, and SEQ ID NO:10 or a portion thereof;
b) a Staufen protein or portion thereof having a sequence selected from the group consisting of: SEQ ID NO:5, SEQ ID NO:1, SEQ ID NO:7, SEQ ID NO:3, and SEQ ID NO:6;
c) an antisense oligonucleotide, comprising at least 10 contiguous nucleotides at least 95% complementary to anyone of SEQ ID NO:5, SEQ ID NO:1, SEQ ID NO:7, SEQ ID NO:3, SEQ ID NO:10, and SEQ ID NO:6; and
d) a siRNA that down regulates Staufen expression.

24. The pharmaceutical composition of claim 23, wherein said siRNA is an siRNA of claims 1-13.

25. A Chimeric protein capable of being incorporated into a HIV-1 or HIV-2 virion, comprising a first and a second portion, wherein said first portion encodes a Staufen protein or portion thereof capable of binding viral double stranded RNA and selected from the group consisting of: SEQ ID NO:2, SEQ ID:4, SEQ ID NO: 10, and SEQ ID NO: 8.

26. The chimeric protein of claim 25, wherein said second portion is a polypeptide covalently attached to the N- or C-terminus of said first portion.

27. The chimeric protein of claim 26, wherein said polypeptide fragment comprises an amino acid sequence which prevents proper virion morphogenesis or assembly of said HIV-1 or HIV-2 virion.

28. A vaccine to treat or prevent a RNA virus infection in a human comprising, a mammalian Staufen protein or fragment thereof selected from the group consisting of SEQ ID NO:2, SEQ ID:4, SEQ ID NO: 10, and SEQ ID NO: 8, or a nucleic acid sequence encoding said Staufen protein or fragment thereof, whereby upon administration of an effective amount of said vaccine to said human an immune reaction is ellicited thereby treating or preventing said RNA virus infection.

29. The vaccine of claim 28, further comprising a second immunogenic peptide or nucleotide sequence encoding said immunogenic peptide to increase its immunological efficiency in human.

30. The siRNA of claim 3, comprising nucleotides 505-568 of SEQ ID NO:56.
